(19) United States
(12) Patent Application Publication
(10) Pub. No.: US 2018/0222966 A1
(43) Pub. Date: Aug. 9, 2018

(54) TRANSGENIC PLANT COMPRISING A POLYNUCLEOTIDE ENCODING A VARIABLE DOMAIN OF HEAVY-CHAIN ANTIBODY

(71) Applicant: AGROSAVFE N.V., Gent (BE)

(72) Inventors: Peter Verheesen, Mariakerke (BE); Chris De Jonghe, Mortsel (BE); Jan Geerinck, Nieuwkerken-Waas (BE); Inge Elodie Van Daele, Melle (BE); Miguel Francesco Coleta De Bolle, Baarle-Nassau (NL); João Filipe Veloso Vieira, Didcot (GB); Marnix Peferoen, Bellem (BE); Karin Thevissen, Bierbeek (BE); Bruno Cammue, Alsemberg (DE)

(21) Appl. No.: 15/926,449

(22) Filed: Mar. 20, 2018

Related U.S. Application Data
(63) Continuation-in-part of application No. 15/524,609, filed on May 4, 2017.
Application No. 15/524,609 is a continuation-in-part of application No. 15/724,840, filed on Oct. 4, 2017.
Application No. 15/724,840 is a division of application No. 14/787,734, filed on Oct. 28, 2015, now Pat. No. 9,803,003.
(60) Provisional application No. 61/817,170, filed on Apr. 29, 2013.

(30) Foreign Application Priority Data
Nov. 5, 2014 (EP) 14191959.7

(51) Int. Cl. C07K 16/14; A01N 63/02; A01N 37/46; C12N 15/82

(52) U.S. Cl. CPC C07K 16/14; A01N 63/02; A01N 37/46; C12N 15/8279; C07K 2317/565; C07K 2317/56; C07K 2317/569; C07K 2317/76; C07K 2317/22

(57) ABSTRACT

This disclosure relates to a transgenic plant or plant tissue. In particular, this disclosure relates to a transgenic plant or plant tissue or plant cell comprising at least one polynucleotide comprising at least one sequence encoding a variable domain of a heavy-chain antibody (VHH) specifically binding to a sphingolipid of a fungus. Advantageously, the expression of the polynucleotide in at least part of the transgenic plant or plant tissue or plant cell (i) protects at least part of the transgenic plant or plant tissue or plant cell from an infection with a plant pathogenic fungus, (ii) inhibits the growth of a plant pathogenic fungus on at least part of the transgenic plant or plant tissue or plant cell, or (iii) increases the resistance of at least part of the transgenic plant or plant tissue or plant cell against a plant pathogenic fungus. This disclosure also relates to a method for protecting at least part of a plant or plant tissue or plant cell from an infection with a plant pathogen, for inhibiting the growth of a plant pathogen on at least part of a plant or plant tissue or plant cell, or for increasing pathogen resistance of at least part of a plant or plant tissue or plant cell, comprising expressing in at least part of the plant or plant tissue or plant cell at least one polynucleotide encoding a VHH specifically binding to a pathogen.

### CROSS-REFERENCE TO RELATED APPLICATIONS

This application is a continuation-in-part of U.S. patent application Ser. No. 15/524,609, filed May 4, 2017, pending, which is a national phase entry under 35 U.S.C. § 371 of International Patent Application PCT/EP2015/075800, filed Nov. 5, 2015, designating the United States of America and published in English as International Patent Publication WO 2016/071438 A2 on May 12, 2016, which claims the benefit under Article 8 of the Patent Cooperation Treaty to European Patent Application Serial No. 14191959.7, filed Nov. 5, 2014, the disclosure of each of which is hereby incorporated herein in its entirety by this reference. This application is also a continuation-in-part of U.S. patent application Ser. No. 15/724,840, filed Oct. 4, 2017, pending, which is a divisional of U.S. patent application Ser. No. 14/787,734, filed Oct. 28, 2015, now U.S. Pat. No. 9,803,003, issued Oct. 31, 2017, which is a national phase entry under 35 U.S.C. § 371 of International Patent Application PCT/EP2014/058772, filed Apr. 29, 2014, designating the United States of America and published in English as International Patent Publication WO 2014/191146 A1 on Dec. 4, 2014. which claims the benefit under Article 8 of the Patent Cooperation Treaty and under 35 U.S.C. § 119(e) to U.S. Provisional Patent Application Ser. No. 61/817,170, filed Apr. 29, 2013, the disclosure of each of which is hereby incorporated herein in its entirety by this reference.

### TECHNICAL FIELD

This application relates to the field of transgenic plants. In particular, this disclosure relates to a transgenic plant or plant tissue or plant cell comprising at least one polynucleotide comprising at least one sequence encoding a variable domain of a heavy-chain antibody (VHH) specifically binding to a sphingolipid of a fungus. This disclosure further relates to a method for protecting at least part of a plant or plant tissue or plant cell from an infection with a plant pathogen, for inhibiting the growth of a plant pathogen on at least part of a plant or plant tissue or plant cell, or for increasing pathogen resistance of at least part of a plant or plant tissue or plant cell, comprising expressing in at least part of the plant or plant tissue or plant cell at least one polynucleotide encoding a VHH specifically binding to a pathogen.

### STATEMENT ACCORDING TO 37 C.F.R. § 1.821(C) OR (E)—SEQUENCE LISTING SUBMITTED AS ASCII TEXT FILE

Pursuant to 37 C.F.R. § 1.821(c) or (e), a file containing an ASCII text version of the Sequence Listing has been submitted concomitant with this application, the contents of which are hereby incorporated by reference.

### BACKGROUND

Crop protection, required for effective agriculture, relies heavily on the use of pesticides, which are applied to the crops by spraying them onto the crop, applying during watering of the crops or incorporating them into the soil. Pesticides are often organic chemical molecules and their repeated application to crops poses toxicity threats to both agricultural workers during handling and to the environment, due to spray drift, persistence in the soil or washing off into surface or ground water. It would be advantageous to be able to use alternative compounds that are less toxic to humans and the environment, but that at the same time provide effective control of plant pests. Proteinaceous pesticides with specificity against a certain plant pest target may be very advantageous in this respect, as they are expected to be short-lived in the environment and to have less toxic off-target effects. However, there are only a few proteinaceous or peptidergic pesticides known. Some examples are Bt toxins, lectins, defensins, fabatins, tachyplesin, magainin, harpin (see WO 2010/019442), pea albumin 1-subunit b (PA1b). However, these proteinaceous pesticides are either small peptides with compact structures, stabilized by several disulphide bridges, or are larger proteins (>300 amino acids) that occur in crystalline form (cry toxins). It is indeed known in the field of agriculture that biologicals and, in particular, proteins, are challenging structures for developing pesticides, as they generally have far too little stability to maintain their pesticidal function in an agrochemical formulation, in particular, for applications in the field.

### BRIEF SUMMARY

The applicants herein have successfully developed transgenic plants comprising a polynucleotide encoding polypeptides with surprisingly high specificity, affinity and potency against targets of plant or crop pests, in particular, plant pathogens, such as plant pathogenic fungi. Moreover, it is shown that these polypeptides retain their integrity, stability and activity upon in planta expression and that efficacious pest or pathogenic control can surprisingly be achieved.

The applicants herein have realized a transgenic plant comprising a polynucleotide, wherein the expression of the polynucleotide in at least part of the transgenic plant (i.e., in planta expression of the polynucleotide) protects at least part of the transgenic plant from an infection or other biological interaction with a plant pathogenic fungus.

Hence, a first aspect of this disclosure relates to a transgenic plant or plant tissue or plant cell comprising at least one polynucleotide comprising at least one sequence encoding a variable domain of a heavy-chain antibody (VHH) specifically binding to a sphingolipid of a fungus.

In certain embodiments of the transgenic plants or plant tissues or plant cells, methods, or uses, as taught herein, a VHH as taught herein specifically binds to a sphingolipid of a fungus. In certain embodiments, a VHH as taught herein specifically binds to a ceramide of a fungus. In certain embodiments, a VHH as taught herein specifically binds to a glycosphingolipid of a fungus. In certain embodiments, a VHH as taught herein specifically binds to a cerebroside of a fungus. In certain preferred embodiments, a VHH as taught herein specifically binds to a glucocerebroside of a fungus.

The applicants herein have found that a VHH as taught herein specifically binds to a sphingolipid of a fungus and binds to a sphingolipid of a plant pathogenic fungus.

Hence, in certain embodiments, this disclosure relates to a transgenic plant or plant tissue or plant cell comprising at least one polynucleotide comprising at least one sequence encoding a VHH specifically binding to a sphingolipid of a fungus, preferably to a ceramide of a fungus, preferably to a glycosphingolipid of a fungus, more preferably to a cerebroside of a fungus, even more preferably to a glucocerebroside of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, this disclosure relates to a transgenic plant or plant tissue or plant cell comprising at least one polynucleotide comprising at least one sequence encoding a VHH specifically binding to a sphingolipid of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, this disclosure relates to a transgenic plant or plant tissue or plant cell comprising at least one polynucleotide comprising at least one sequence encoding a VHH specifically binding to a ceramide of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, this disclosure relates to a transgenic plant or plant tissue or plant cell comprising at least one polynucleotide comprising at least one sequence encoding a VHH specifically binding to a glycosphingolipid of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, this disclosure relates to a transgenic plant or plant tissue or plant cell comprising at least one polynucleotide comprising at least one sequence encoding a VHH specifically binding to a cerebroside of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, this disclosure relates to a transgenic plant or plant tissue or plant cell comprising at least one polynucleotide comprising at least one sequence encoding a VHH specifically binding to a glucocerebroside of a fungus, and wherein the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments of the transgenic plants or plant tissues or plant cells, methods, or uses, as taught herein, a VHH as taught herein binds to a sphingolipid of a plant pathogenic fungus. In certain embodiments, a VHH as taught herein binds to a ceramide of a plant pathogenic fungus. In certain embodiments, a VHH as taught herein specifically binds to a glycosphingolipid of a plant pathogenic fungus. In certain embodiments, a VHH as taught herein specifically binds to a cerebroside of a plant pathogenic fungus. In certain preferred embodiments, a VHH as taught herein specifically binds to a glucocerebroside of a plant pathogenic fungus.

In certain embodiments, the transgenic plant or plant tissue or plant cell may have an increased or enhanced level of a VHH as taught herein relative to (i.e., compared with) a non-modified (i.e., non-transformed or untransformed, such as wild-type) plant or plant tissue.

In certain embodiments, the transgenic plant or plant tissue or plant cell may have a level of a VHH as taught herein, which is at least 0.001% of the amount of total soluble protein in the transgenic plant or plant tissue or plant cell, in particular, in an extract of the transgenic plant or plant tissue. For example, the transgenic plant or plant tissue or plant cell may have a level of a VHH as taught herein, which is at least 0.005%, at least 0.01%, at least 0.05%, at least 0.1%, at least 0.2%, at least 0.3%, at least 0.4%, or at least 0.5% of the amount of total soluble protein in the transgenic plant or plant tissue or plant cell, in particular, in an extract of the transgenic plant or plant tissue.

In certain embodiments, the expression of the polynucleotide in at least part of the transgenic plant or plant tissue or plant cell may protect at least part of the transgenic plant or plant tissue or plant cell from an infection with a plant pathogenic fungus. For example, the expression of the polynucleotide in at least part of the transgenic plant or plant tissue or plant cell may protect at least part of the transgenic plant or plant tissue or plant cell from an infection with a plant pathogenic fungus relative to (i.e., compared with) a non-modified (i.e., non-transformed or untransformed, such as wild-type) plant or plant tissue.

In certain embodiments, the expression of the polynucleotide in at least part of the transgenic plant or plant tissue or plant cell may inhibit the growth of a plant pathogenic fungus on at least part of the transgenic plant or plant tissue. For example, the expression of the polynucleotide in at least part of the transgenic plant or plant tissue or plant cell may inhibit the growth of a plant pathogenic fungus on at least part of the transgenic plant or plant tissue or plant cell relative to (i.e., compared with) the growth of the plant pathogenic fungus on a non-modified (i.e., non-transformed or untransformed, such as wild-type) plant or plant tissue.

In certain embodiments, the expression of the polynucleotide in at least part of the transgenic plant or plant tissue or plant cell may increases the resistance of at least part of the transgenic plant or plant tissue or plant cell against a plant pathogenic fungus. For example, the expression of the polynucleotide in at least part of the transgenic plant or plant tissue or plant cell may increases the resistance of at least part of the transgenic plant or plant tissue or plant cell against a plant pathogenic fungus relative to (i.e., compared with) the resistance of a non-modified (i.e., non-transformed or untransformed, such as wild-type) plant or plant tissue or plant cell against the plant pathogenic fungus.

In certain embodiments, the expression of the polynucleotide in at least part of the transgenic plant or plant tissue or plant cell may protect at least part of the transgenic plant or plant tissue or plant cell from an infection with a plant pathogenic fungus, may inhibit the growth of a plant pathogenic fungus on at least part of the transgenic plant or plant tissue or plant cell, and/or may increase the resistance of at least part of the transgenic plant or plant tissue or plant cell against a plant pathogenic fungus.

In certain embodiments of the transgenic plants or plant tissues or plant cells, methods, or uses, as taught herein, the polynucleotide may comprise a promoter suitable for expression in plants, such as the 35S Cauliflower Mosaic Virus (CaMV) promoter, a plant tissue or plant cell specific promoter, or an inducible promoter.

In certain embodiments of the transgenic plants or plant tissues or plant cells, methods, or uses, as taught herein, the polynucleotide may comprise at least one sequence encoding a targeting signal for secretion, for location to the cytoplasm, or for location to cellular compartments or organelles, such as the ER lumen, the apoplast, the vacuole, or intra- and/or exterior membranes.

In certain embodiments of the transgenic plants or plant tissues or plant cells, methods, or uses, as taught herein, the polynucleotide may comprise at least one sequence encoding a tag, preferably a His6, c-myc, FLAG, C-tag, 3×FLAG, His5, His10, HA, T7, strep, HSV, and/or an E-tag.

In certain embodiments of the transgenic plants or plant tissues or plant cells, methods, or uses, as taught herein, the polynucleotide may encode the VHH as such, as a combination with one or more identical or different VHHs, or as a combination with one or more identical or different VHHs with an fragment crystallizable region (Fc region) of an antibody; optionally with a spacer.

In certain embodiments, the polynucleotide may encode the VHH as such, optionally with a spacer. In certain other embodiments, the polynucleotide may encode the VHH as a combination with one or more, such as two or more, identical or different VHHs, optionally with a spacer. In certain other embodiments, the polynucleotide may encode the VHH as a combination with one or more, such as two or more, identical or different VHHs with a fragment crystallizable region (Fc region) of an antibody, optionally with a spacer. Such a spacer advantageously spatially extends two VHHs from each other, thereby enhancing the flexibility of the VHHs relative to each other and/or assuring optimal interaction between each VHH and its antigen.

In certain embodiments of the transgenic plants or plant tissues or plant cells, methods, or uses, as taught herein, the plant may be a plant selected from the group consisting of corn, rice, wheat, barley, sorghum, millet oats, rye, triticale or other cereals, soybean, alfalfa or other leguminous crops, sugar beet, fodder beet, papaya, banana and plantains or other fruits, grapevines, nuts, oilseed rape, sunflower or other oil crops, squash cucumber, melons or other cucurbits, cotton or other fiber plants, sugarcane, palm, jatropha or other fuel crops, cabbages, tomato, pepper or other vegetables, ornamentals, shrubs, poplar, eucalyptus or other trees, evergreens, grasses, coffee plants, tea plants, tobacco plants, hop plants, rubber plants, and latex plants.

In certain embodiments of the transgenic plants or plant tissues or plant cells, methods, or uses, as taught herein, the polynucleotide may comprise a sequence encoding a VHH comprising:
(i) any one or more of SEQ ID NOS:1 to 84, preferably SEQ ID NOS:1, 2, and/or 70, more preferably SEQ ID NOS:1 and/or 2, and/or
(ii) a CDR1, CDR2, and CDR3 region, wherein (i) the CDR1 region is selected from the group of SEQ ID NOS:85-168, and/or (ii) the CDR2 region is selected from the group of SEQ ID NOS:169-252, and/or (iii) the CDR3 region is selected from the group of SEQ ID NOS:253-335, or the CDR3 region has the amino acid sequence NRY.

In certain embodiments of the transgenic plants or plant tissues or plant cells, methods, or uses, as taught herein, the polynucleotide may comprise a sequence encoding a VHH comprising any one or more of SEQ ID NOS:1 to 84, preferably SEQ ID NOS:1 and/or 2. In certain embodiments, the polynucleotide may comprise a sequence encoding a VHH comprising SEQ ID NO:1, SEQ ID NO:2, and/or SEQ ID NO:70. In certain embodiments, the polynucleotide may comprise a sequence encoding a VHH comprising SEQ ID NO:1 and/or SEQ ID NO:2. In certain embodiments, the polynucleotide may comprise a sequence encoding a VHH comprising SEQ ID NO:1. In certain embodiments, the polynucleotide may comprise a sequence encoding a VHH comprising SEQ ID NO:2. In certain embodiments, the polynucleotide may comprise a sequence encoding a VHH comprising SEQ ID NO:70.

In certain embodiments of the transgenic plants or plant tissues or plant cells, methods, or uses, as taught herein, the polynucleotide may comprise a sequence encoding a VHH comprising a CDR1, CDR2, and CDR3 region, wherein (i) the CDR1 region is selected from the group of SEQ ID NOS:85-168, and/or (ii) the CDR2 region is selected from the group of SEQ ID NOS:169-252, and/or (iii) the CDR3 region is selected from the group of SEQ ID NOS:253-335, or the CDR3 region has the amino acid sequence NRY.

In certain embodiments, the polynucleotide may comprise a sequence encoding a VHH comprising a CDR1, CDR2, and CDR3 region, wherein (i) the CDR1 region is selected from SEQ ID NOS:85 and/or 86, and/or (ii) the CDR2 region is selected from SEQ ID NOS:169 and/or 170, and/or (iii) the CDR3 region is selected from SEQ ID NOS:253 and/or 254. In certain embodiments, the polynucleotide may comprise a sequence encoding a VHH comprising a CDR1, CDR2, and CDR3 region, wherein (i) the CDR1 region is SEQ ID NO:85, and/or (ii) the CDR2 region is SEQ ID NO:169, and/or (iii) the CDR3 region is SEQ ID NO:253. In certain embodiments, the polynucleotide may comprise a sequence encoding a VHH comprising a CDR1, CDR2, and CDR3 region, wherein (i) the CDR1 region is SEQ ID NO:86, and/or (ii) the CDR2 region is SEQ ID NO:170, and/or (iii) the CDR3 region is SEQ ID NO:254.

In certain embodiments of the transgenic plants or plant tissues or plant cells, methods, or uses, as taught herein, the polynucleotide may comprise a sequence encoding a VHH comprising, consisting of, or consisting essentially of four framework regions (FRs) and three complementarity-determining regions (CDRs), a CDR1, CDR2, and CDR3 region, wherein (i) the CDR1 region is selected from the group of SEQ ID NOS:85-168, and/or (ii) the CDR2 region is selected from the group of SEQ ID NOS:169-252, and/or (iii) the CDR3 region is selected from the group of SEQ ID NOS:253-335, or the CDR3 region has the amino acid sequence NRY.

In certain embodiments, the polynucleotide may comprise a sequence encoding a VHH comprising four framework regions and three CDRs, a CDR1, CDR2, and CDR3 region, wherein (i) the CDR1 region is selected from SEQ ID NOS:85 and/or 86, and/or (ii) the CDR2 region is selected from SEQ ID NOS:169 and/or 170, and/or (iii) the CDR3 region is selected from SEQ ID NOS:253 and/or 254. In certain embodiments, the polynucleotide may comprise a sequence encoding a VHH comprising four framework regions and three CDRs, a CDR1, CDR2, and CDR3 region, wherein (i) the CDR1 region is SEQ ID NO:85, and/or (ii) the CDR2 region is SEQ ID NO:169, and/or (iii) the CDR3 region is SEQ ID NO:253. In certain embodiments, the polynucleotide may comprise a sequence encoding a VHH comprising four framework regions and three CDRs, a CDR1, CDR2, and CDR3 region, wherein (i) the CDR1 region is SEQ ID NO:86, and/or (ii) the CDR2 region is SEQ ID NO:170, and/or (iii) the CDR3 region is SEQ ID NO:254.

In certain embodiments of the transgenic plants or plant tissues or plant cells, methods, or uses, as taught herein, the polynucleotide may comprise a sequence encoding a VHH comprising a CDR1, CDR2 and CDR3 region chosen from the list of comprising:
a CDR1 region having SEQ ID NO:85, a CDR2 region having has SEQ ID NO:169, and a CDR3 region having SEQ ID NO:253, and/or
a CDR1 region having SEQ ID NO:86, a CDR2 region having has SEQ ID NO:170, and a CDR3 region having SEQ ID NO:254, and/or
a CDR1 region having SEQ ID NO:87, a CDR2 region having has SEQ ID NO:171, and a CDR3 region having SEQ ID NO:255, and/or
a CDR1 region having SEQ ID NO:88, a CDR2 region having has SEQ ID NO:172, and a CDR3 region having SEQ ID NO:256, and/or
a CDR1 region having SEQ ID NO:89, a CDR2 region having has SEQ ID NO:173, and a CDR3 region having SEQ ID NO:257, and/or
a CDR1 region having SEQ ID NO:90, a CDR2 region having has SEQ ID NO:174, and a CDR3 region having SEQ ID NO:258, and/or
a CDR1 region having SEQ ID NO:91, a CDR2 region having has SEQ ID NO:175, and a CDR3 region having SEQ ID NO:259, and/or
a CDR1 region having SEQ ID NO:92, a CDR2 region having has SEQ ID NO:176, and a CDR3 region having SEQ ID NO:260, and/or
a CDR1 region having SEQ ID NO:93, a CDR2 region having has SEQ ID NO:177, and a CDR3 region having SEQ ID NO:261, and/or
a CDR1 region having SEQ ID NO:94, a CDR2 region having has SEQ ID NO:178, and a CDR3 region having SEQ ID NO:262, and/or
a CDR1 region having SEQ ID NO:95, a CDR2 region having has SEQ ID NO:179, and a CDR3 region having SEQ ID NO:263, and/or
a CDR1 region having SEQ ID NO:96, a CDR2 region having has SEQ ID NO:180, and a CDR3 region having SEQ ID NO:264, and/or
a CDR1 region having SEQ ID NO:97, a CDR2 region having has SEQ ID NO:181, and a CDR3 region having SEQ ID NO:265, and/or
a CDR1 region having SEQ ID NO:98, a CDR2 region having has SEQ ID NO:182, and a CDR3 region having SEQ ID NO:266, and/or
a CDR1 region having SEQ ID NO:99, a CDR2 region having has SEQ ID NO:183, and a CDR3 region having SEQ ID NO:267, and/or
a CDR1 region having SEQ ID NO:100, a CDR2 region having has SEQ ID NO:184, and a CDR3 region having SEQ ID NO:268, and/or
a CDR1 region having SEQ ID NO:101, a CDR2 region having has SEQ ID NO:185, and a CDR3 region having SEQ ID NO:269, and/or
a CDR1 region having SEQ ID NO:102, a CDR2 region having has SEQ ID NO:186, and a CDR3 region having SEQ ID NO:270, and/or
a CDR1 region having SEQ ID NO:103, a CDR2 region having has SEQ ID NO:187, and a CDR3 region having SEQ ID NO:271, and/or
a CDR1 region having SEQ ID NO:104, a CDR2 region having has SEQ ID NO:188, and a CDR3 region having SEQ ID NO:272, and/or
a CDR1 region having SEQ ID NO:105, a CDR2 region having has SEQ ID NO:189, and a CDR3 region having SEQ ID NO:273, and/or
a CDR1 region having SEQ ID NO:106, a CDR2 region having has SEQ ID NO:190, and a CDR3 region having SEQ ID NO:274, and/or
a CDR1 region having SEQ ID NO:107, a CDR2 region having has SEQ ID NO:191, and a CDR3 region having SEQ ID NO:275, and/or
a CDR1 region having SEQ ID NO:108, a CDR2 region having has SEQ ID NO:192, and a CDR3 region having SEQ ID NO:276, and/or
a CDR1 region having SEQ ID NO:109, a CDR2 region having has SEQ ID NO:193, and a CDR3 region having SEQ ID NO:277, and/or
a CDR1 region having SEQ ID NO:110, a CDR2 region having has SEQ ID NO:194, and a CDR3 region having SEQ ID NO:278, and/or
a CDR1 region having SEQ ID NO:111, a CDR2 region having has SEQ ID NO:195, and a CDR3 region having SEQ ID NO:279, and/or
a CDR1 region having SEQ ID NO:112, a CDR2 region having has SEQ ID NO:196, and a CDR3 region having SEQ ID NO:280, and/or
a CDR1 region having SEQ ID NO:113, a CDR2 region having has SEQ ID NO:197, and a CDR3 region having SEQ ID NO:281, and/or
a CDR1 region having SEQ ID NO:114, a CDR2 region having has SEQ ID NO:198, and a CDR3 region having SEQ ID NO:282, and/or
a CDR1 region having SEQ ID NO:115, a CDR2 region having has SEQ ID NO:199, and a CDR3 region having SEQ ID NO:283, and/or
a CDR1 region having SEQ ID NO:116, a CDR2 region having has SEQ ID NO:200, and a CDR3 region having SEQ ID NO:284, and/or
a CDR1 region having SEQ ID NO:117, a CDR2 region having has SEQ ID NO:201, and a CDR3 region having SEQ ID NO:285, and/or
a CDR1 region having SEQ ID NO:118, a CDR2 region having has SEQ ID NO:202, and a CDR3 region having SEQ ID NO:286, and/or
a CDR1 region having SEQ ID NO:119, a CDR2 region having has SEQ ID NO:203, and a CDR3 region having SEQ ID NO:287, and/or
a CDR1 region having SEQ ID NO:120, a CDR2 region having has SEQ ID NO:204, and a CDR3 region having SEQ ID NO:288, and/or
a CDR1 region having SEQ ID NO:121, a CDR2 region having has SEQ ID NO:205, and a CDR3 region having SEQ ID NO:289, and/or
a CDR1 region having SEQ ID NO:122, a CDR2 region having has SEQ ID NO:206, and a CDR3 region having SEQ ID NO:290, and/or
a CDR1 region having SEQ ID NO:123, a CDR2 region having has SEQ ID NO:207, and a CDR3 region having SEQ ID NO:291, and/or
a CDR1 region having SEQ ID NO:124, a CDR2 region having has SEQ ID NO:208, and a CDR3 region having SEQ ID NO:292, and/or
a CDR1 region having SEQ ID NO:125, a CDR2 region having has SEQ ID NO:209, and a CDR3 region having SEQ ID NO:293, and/or
a CDR1 region having SEQ ID NO:126, a CDR2 region having has SEQ ID NO:210, and a CDR3 region having SEQ ID NO:294, and/or
a CDR1 region having SEQ ID NO:127, a CDR2 region having has SEQ ID NO:211, and a CDR3 region having SEQ ID NO:295, and/or
a CDR1 region having SEQ ID NO:128, a CDR2 region having has SEQ ID NO:212, and a CDR3 region having SEQ ID NO:296, and/or
a CDR1 region having SEQ ID NO:129, a CDR2 region having has SEQ ID NO:213, and a CDR3 region having SEQ ID NO:297, and/or
a CDR1 region having SEQ ID NO:130, a CDR2 region having has SEQ ID NO:214, and a CDR3 region having SEQ ID NO:298, and/or
a CDR1 region having SEQ ID NO:131, a CDR2 region having has SEQ ID NO:215, and a CDR3 region having SEQ ID NO:299, and/or
a CDR1 region having SEQ ID NO:132, a CDR2 region having has SEQ ID NO:216, and a CDR3 region having SEQ ID NO:300, and/or
a CDR1 region having SEQ ID NO:133, a CDR2 region having has SEQ ID NO:217, and a CDR3 region having SEQ ID NO:301, and/or
a CDR1 region having SEQ ID NO:134, a CDR2 region having has SEQ ID NO:218, and a CDR3 region having SEQ ID NO:302, and/or
a CDR1 region having SEQ ID NO:135, a CDR2 region having has SEQ ID NO:219, and a CDR3 region having SEQ ID NO:303, and/or
a CDR1 region having SEQ ID NO:136, a CDR2 region having has SEQ ID NO:220, and a CDR3 region having SEQ ID NO:304, and/or
a CDR1 region having SEQ ID NO:137, a CDR2 region having has SEQ ID NO:221, and a CDR3 region having SEQ ID NO:305, and/or
a CDR1 region having SEQ ID NO:138, a CDR2 region having has SEQ ID NO:222, and a CDR3 region having SEQ ID NO:306, and/or
a CDR1 region having SEQ ID NO:139, a CDR2 region having has SEQ ID NO:223, and a CDR3 region having the amino acid sequence NRY, and/or
a CDR1 region having SEQ ID NO:140, a CDR2 region having has SEQ ID NO:224, and a CDR3 region having SEQ ID NO:307, and/or
a CDR1 region having SEQ ID NO:141, a CDR2 region having has SEQ ID NO:225, and a CDR3 region having SEQ ID NO:308, and/or
a CDR1 region having SEQ ID NO:142, a CDR2 region having has SEQ ID NO:226, and a CDR3 region having SEQ ID NO:309, and/or
a CDR1 region having SEQ ID NO:143, a CDR2 region having has SEQ ID NO:227, and a CDR3 region having SEQ ID NO:310, and/or
a CDR1 region having SEQ ID NO:144, a CDR2 region having has SEQ ID NO:228, and a CDR3 region having SEQ ID NO:311, and/or
a CDR1 region having SEQ ID NO:145, a CDR2 region having has SEQ ID NO:229, and a CDR3 region having SEQ ID NO:312, and/or
a CDR1 region having SEQ ID NO:146, a CDR2 region having has SEQ ID NO:230, and a CDR3 region having SEQ ID NO:313, and/or
a CDR1 region having SEQ ID NO:147, a CDR2 region having has SEQ ID NO:231, and a CDR3 region having SEQ ID NO:314, and/or
a CDR1 region having SEQ ID NO:148, a CDR2 region having has SEQ ID NO:232, and a CDR3 region having SEQ ID NO:315, and/or
a CDR1 region having SEQ ID NO:149, a CDR2 region having has SEQ ID NO:233, and a CDR3 region having SEQ ID NO:316, and/or
a CDR1 region having SEQ ID NO:150, a CDR2 region having has SEQ ID NO:234, and a CDR3 region having SEQ ID NO:317, and/or
a CDR1 region having SEQ ID NO:151, a CDR2 region having has SEQ ID NO:235, and a CDR3 region having SEQ ID NO:318, and/or
a CDR1 region having SEQ ID NO:152, a CDR2 region having has SEQ ID NO:236, and a CDR3 region having SEQ ID NO:319, and/or
a CDR1 region having SEQ ID NO:153, a CDR2 region having has SEQ ID NO:237, and a CDR3 region having SEQ ID NO:320, and/or
a CDR1 region having SEQ ID NO:154, a CDR2 region having has SEQ ID NO:238, and a CDR3 region having SEQ ID NO:321, and/or
a CDR1 region having SEQ ID NO:155, a CDR2 region having has SEQ ID NO:239, and a CDR3 region having SEQ ID NO:322, and/or
a CDR1 region having SEQ ID NO:156, a CDR2 region having has SEQ ID NO:240, and a CDR3 region having SEQ ID NO:323, and/or
a CDR1 region having SEQ ID NO:157, a CDR2 region having has SEQ ID NO:241, and a CDR3 region having SEQ ID NO:324, and/or
a CDR1 region having SEQ ID NO:158, a CDR2 region having has SEQ ID NO:242, and a CDR3 region having SEQ ID NO:325, and/or
a CDR1 region having SEQ ID NO:159, a CDR2 region having has SEQ ID NO:243, and a CDR3 region having SEQ ID NO:326, and/or
a CDR1 region having SEQ ID NO:160, a CDR2 region having has SEQ ID NO:244, and a CDR3 region having SEQ ID NO:327, and/or
a CDR1 region having SEQ ID NO:161, a CDR2 region having has SEQ ID NO:245, and a CDR3 region having SEQ ID NO:328, and/or
a CDR1 region having SEQ ID NO:162, a CDR2 region having has SEQ ID NO:246, and a CDR3 region having SEQ ID NO:329, and/or
a CDR1 region having SEQ ID NO:163, a CDR2 region having has SEQ ID NO:247, and a CDR3 region having SEQ ID NO:330, and/or
a CDR1 region having SEQ ID NO:164, a CDR2 region having has SEQ ID NO:248, and a CDR3 region having SEQ ID NO:331, and/or
a CDR1 region having SEQ ID NO:165, a CDR2 region having has SEQ ID NO:249, and a CDR3 region having SEQ ID NO:332, and/or
a CDR1 region having SEQ ID NO:166, a CDR2 region having has SEQ ID NO:250, and a CDR3 region having SEQ ID NO:333, and/or
a CDR1 region having SEQ ID NO:167, a CDR2 region having has SEQ ID NO:251, and a CDR3 region having SEQ ID NO:334, and/or
a CDR1 region having SEQ ID NO:168, a CDR2 region having has SEQ ID NO:252, and a CDR3 region having SEQ ID NO:335.

In certain embodiments of the transgenic plants or plant tissues or plant cells, methods, or uses, as taught herein, the polynucleotide may comprise a sequence encoding a VHH comprising a CDR1, CDR2 and CDR3 region chosen from the list of comprising:
a CDR1 region having SEQ ID NO:85, a CDR2 region having has SEQ ID NO:169, and a CDR3 region having SEQ ID NO:253, and/or
a CDR1 region having SEQ ID NO:86, a CDR2 region having has SEQ ID NO:170, and a CDR3 region having SEQ ID NO:254.

In certain embodiments of the transgenic plants or plant tissues or plant cells, methods, or uses, as taught herein, the polynucleotide may comprise a sequence encoding a VHH comprising a CDR1 region having SEQ ID NO:85, a CDR2 region having has SEQ ID NO:169, and a CDR3 region having SEQ ID NO:253.

In certain embodiments of the transgenic plants or plant tissues or plant cells, methods, or uses, as taught herein, the polynucleotide may comprise a sequence encoding a VHH comprising a CDR1 region having SEQ ID NO:86, a CDR2 region having has SEQ ID NO:170, and a CDR3 region having SEQ ID NO:254.

A further aspect relates to harvestable parts and propagation materials of a transgenic plant or plant tissue or plant cell as defined herein, comprising at least one polynucleotide as defined herein. Accordingly, a further aspect provides harvestable parts and propagation materials of a transgenic plant or plant tissue or plant cell as defined herein, comprising at least one polynucleotide comprising at least one sequence encoding a VHH specifically binding to a sphingolipid of a fungus, preferably to a ceramide of a fungus, preferably to a glycosphingolipid of a fungus, more preferably to a cerebroside of a fungus, even more preferably to a glucocerebroside of a fungus. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

In certain embodiments, the harvestable parts and propagation materials of a transgenic plant or plant tissue or plant cell are selected from the group consisting of seeds, fruits, grains, bulbs, bolls, tubers, progeny, and hybrids.

A further aspect relates to an extract of a transgenic plant or plant tissue or plant cell as defined herein, comprising at least one polynucleotide as defined herein, the extract comprising VHH. Accordingly, a further aspect provides an extract of a transgenic plant or plant tissue or plant cell as defined herein, comprising at least one VHH specifically binding to a sphingolipid of a fungus, preferably to a ceramide of a fungus, preferably to a glycosphingolipid of a fungus, more preferably to a cerebroside of a fungus, even more preferably to a glucocerebroside of a fungus. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

A further aspect relates to compositions comprising an extract of a transgenic plant or plant tissue or plant cell as defined herein. Accordingly, a further aspect provides a composition comprising an extract of a transgenic plant or plant tissue or plant cell as defined herein, comprising at least one VHH specifically binding to a sphingolipid of a fungus, preferably to a ceramide of a fungus, preferably to a glycosphingolipid of a fungus, more preferably to a cerebroside of a fungus, even more preferably to a glucocerebroside of a fungus. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

A further aspect relates to a method for the production of a transgenic plant or plant tissue or plant cell comprising the introduction of at least one polynucleotide as defined herein into the genome of a plant or plant tissue. Hence, a further aspect provides a method for the production of a transgenic plant or plant tissue or plant cell comprising the introduction of at least one polynucleotide comprising at least one sequence encoding a VHH specifically binding to a sphingolipid of a fungus into the genome of a plant or plant tissue. In certain embodiments, this disclosure relates to a method for the production of a transgenic plant or plant tissue or plant cell comprising the introduction of at least one polynucleotide comprising at least one sequence encoding a VHH specifically binding to a ceramide of a fungus into the genome of a plant or plant tissue. In certain embodiments, this disclosure relates to a method for the production of a transgenic plant or plant tissue or plant cell comprising the introduction of at least one polynucleotide comprising at least one sequence encoding a VHH specifically binding to a glycosphingolipid of a fungus into the genome of a plant or plant tissue. In certain embodiments, this disclosure relates to a method for the production of a transgenic plant or plant tissue or plant cell comprising the introduction of at least one polynucleotide comprising at least one sequence encoding a VHH specifically binding to a cerebroside of a fungus into the genome of a plant or plant tissue. In certain embodiments, this disclosure relates to a method for the production of a transgenic plant or plant tissue or plant cell comprising the introduction of at least one polynucleotide comprising at least one sequence encoding a VHH specifically binding to a glucocerebroside of a fungus into the genome of a plant or plant tissue. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

A further aspect relates to the use of at least one polynucleotide as defined herein, for the production of a transgenic plant or plant tissue. A further aspect thus provides the use of at least one polynucleotide comprising at least one sequence encoding a VHH specifically binding to a sphingolipid of a fungus, for the production of a transgenic plant or plant tissue. In certain embodiments, the disclosure relates to the use of at least one polynucleotide comprising at least one sequence encoding a VHH specifically binding to a ceramide of a fungus, for the production of a transgenic plant or plant tissue. In certain embodiments, the disclosure relates to the use of at least one polynucleotide comprising at least one sequence encoding a VHH specifically binding to a glycosphingolipid of a fungus, for the production of a transgenic plant or plant tissue. In certain embodiments, the disclosure relates to the use of at least one polynucleotide comprising at least one sequence encoding a VHH specifically binding to a cerebroside of a fungus, for the production of a transgenic plant or plant tissue. In certain embodiments, the disclosure relates to the use of at least one polynucleotide comprising at least one sequence encoding a VHH specifically binding to a glucocerebroside of a fungus, for the production of a transgenic plant or plant tissue. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

The applicants herein have found that a VHH specifically binding to a pathogen (and binding to a plant pathogen), which is produced in at least part of the transgenic plant (e.g., in planta expression of a polynucleotide encoding the VHH specifically binding to a pathogen), has antimicrobial effects per se, in particular, microbiostatic effects, on the plant pathogen. The applicants found that a VHH as taught herein does not act as a “targeting agent” of an antimicrobial substance or composition to the plant pathogen but as an antimicrobial agent per se, in particular, as a microbiostatic agent per se, on the plant pathogen.

Accordingly, a further aspect of this disclosure relates to a method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising expressing in at least part of the plant or plant tissue or plant cell at least one polynucleotide encoding a variable domain of a heavy-chain antibody (VHH) specifically binding to a pathogen. In certain embodiments, the VHH may specifically bind to a sphingolipid of a pathogen, preferably to a ceramide of a pathogen, preferably to a glycosphingolipid of a pathogen, more preferably to a cerebroside of a pathogen, even more preferably to a glucocerebroside of a pathogen.

In certain embodiments, this disclosure relates to a method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising expressing in at least part of the plant or plant tissue or plant cell at least one polynucleotide encoding a VHH specifically binding to a pathogen, and wherein the VHH binds to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

A further aspect of this disclosure relates to a method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising treating at least part of a plant or plant tissue or plant cell with an extract of a transgenic plant or plant tissue or plant cell as defined herein, comprising at least one polynucleotide as defined herein, the extract comprising VHH.

A further aspect of this disclosure relates to a method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising treating at least part of a plant or plant tissue or plant cell with a composition comprising an extract of a transgenic plant or plant tissue or plant cell as defined herein, comprising at least one polynucleotide as defined herein, the extract comprising VHH.

A further aspect relates to the use of an extract of a transgenic plant or plant tissue or plant cell as defined herein, comprising at least one polynucleotide as defined herein, the extract comprising VHH, for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell.

A further aspect relates to the use of a composition comprising an extract of a transgenic plant or plant tissue or plant cell as defined herein, comprising at least one polynucleotide as defined herein, the extract comprising VHH, for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell.

In certain embodiments of the methods or uses, as taught herein, the VHH may specifically bind to a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments of the methods or uses, as taught herein, the VHH may specifically bind to a sphingolipid of a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments of the methods or uses, as taught herein, the VHH may specifically bind to a ceramide of a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments of the methods or uses, as taught herein, the VHH may specifically bind to a glycosphingolipid of a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments of the methods or uses, as taught herein, the VHH may specifically bind to a cerebroside of a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments of the methods or uses, as taught herein, the VHH may specifically bind to a glucocerebroside of a pathogen, and the VHH may bind to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments, this disclosure relates to a method for protecting at least part of a plant or plant tissue or plant cell from an infection or other biological interaction with a plant pathogen, comprising expressing in at least part of the plant or plant tissue or plant cell at least one polynucleotide encoding a VHH specifically binding to a pathogen.

In certain embodiments, this disclosure relates to a method for inhibiting the growth of a plant pathogen on at least part of a plant or plant tissue or plant cell, comprising expressing in at least part of the plant or plant tissue or plant cell at least one polynucleotide encoding a VHH specifically binding to a pathogen.

In certain embodiments, this disclosure relates to a method for increasing pathogen resistance of at least part of a plant or plant tissue or plant cell, comprising expressing in at least part of the plant or plant tissue or plant cell at least one polynucleotide encoding a VHH specifically binding to a pathogen. In certain embodiments, this disclosure relates to a method for increasing resistance of at least part of a plant or plant tissue or plant cell against a plant pathogen, comprising expressing in at least part of the plant or plant tissue or plant cell at least one polynucleotide encoding a VHH specifically binding to a pathogen.

In certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide as defined herein. Hence, in certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide comprising at least one sequence encoding a VHH specifically binding to a sphingolipid of a fungus. In certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide comprising at least one sequence encoding a VHH specifically binding to a ceramide of a fungus. In certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide comprising at least one sequence encoding a VHH specifically binding to a glycosphingolipid of a fungus. In certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide comprising at least one sequence encoding a VHH specifically binding to a cerebroside of a fungus. in certain embodiments, this disclosure relates to a method as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide comprising at least one sequence encoding a VHH specifically binding to a glucocerebroside of a fungus. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

A further aspect relates to the use of at least one polynucleotide encoding a variable domain of a heavy-chain antibody (VHH) specifically binding to a pathogen, for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, wherein the polynucleotide is expressed in at least part of the plant or plant tissue. In certain embodiments, the VHH may specifically bind to a sphingolipid of a pathogen, preferably to a ceramide of a pathogen, preferably to a glycosphingolipid of a pathogen, more preferably to a cerebroside of a pathogen, even more preferably to a glucocerebroside of a pathogen.

In certain embodiments, this disclosure relates to the use of at least one polynucleotide encoding a VHH specifically binding to a pathogen, for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, wherein the polynucleotide is expressed in at least part of the plant or plant tissue or plant cell, and wherein the VHH binds to a plant pathogen, preferably to a sphingolipid of a plant pathogen, preferably to a ceramide of a plant pathogen, preferably to a glycosphingolipid of a plant pathogen, more preferably to a cerebroside of a plant pathogen, even more preferably to a glucocerebroside of a plant pathogen.

In certain embodiments, this disclosure relates to the use of at least one polynucleotide encoding a VHH specifically binding to a pathogen, for protecting at least part of a plant or plant tissue or plant cell from an infection or other biological interaction with a plant pathogen, wherein the polynucleotide is expressed in at least part of the plant or plant tissue.

In certain embodiments, this disclosure relates to the use of at least one polynucleotide encoding a VHH specifically binding to a pathogen, for inhibiting the growth of a plant pathogen on at least part of a plant or plant tissue or plant cell, wherein the polynucleotide is expressed in at least part of the plant or plant tissue.

In certain embodiments, this disclosure relates to the use of at least one polynucleotide encoding a VHH specifically binding to a pathogen, for increasing pathogen resistance of at least part of a plant or plant tissue or plant cell, wherein the polynucleotide is expressed in at least part of the plant or plant tissue. In certain embodiments, this disclosure relates to the use of at least one polynucleotide encoding a VHH specifically binding to a pathogen, for increasing resistance of at least part of a plant or plant tissue or plant cell against a plant pathogen, wherein the polynucleotide is expressed in at least part of the plant or plant tissue.

In certain embodiments, this disclosure relates to the uses as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing pathogen resistance of at least part of a plant or plant tissue or plant cell, wherein the polynucleotide is as defined herein. In certain embodiments, this disclosure relates to the uses as taught herein, for protecting at least part of a plant or plant tissue or plant cell from an infection with a plant pathogenic fungus, for inhibiting the growth of a plant pathogenic fungus on at least part of a plant or plant tissue or plant cell, and/or for increasing pathogen resistance of at least part of a plant or plant tissue or plant cell, wherein the polynucleotide comprises at least one sequence encoding a VHH specifically binding to a sphingolipid of a fungus. In certain embodiments, the polynucleotide may comprise at least one sequence encoding a VHH specifically binding to a ceramide of a fungus. In certain embodiments, the polynucleotide may comprise at least one sequence encoding a VHH specifically binding to a glycosphingolipid of a fungus. In certain embodiments, the polynucleotide may comprise at least one sequence encoding a VHH specifically binding to a cerebroside of a fungus. In certain embodiments, the polynucleotide may comprise at least one sequence encoding a VHH specifically binding to a glucocerebroside of a fungus. In certain embodiments, the VHH binds to a sphingolipid of a plant pathogenic fungus, preferably to a ceramide of a plant pathogenic fungus, preferably to a glycosphingolipid of a plant pathogenic fungus, more preferably to a cerebroside of a plant pathogenic fungus, even more preferably to a glucocerebroside of a plant pathogenic fungus.

The methods and uses embodying the principles of this disclosure advantageously allow protecting a plant or plant tissue or plant cell from an infection or other interaction with a plant pathogen, inhibiting the growth of the plant pathogen on at least part of a plant or plant tissue or plant cell, or increasing pathogen resistance of at least part of a plant or plant tissue or plant cell by the in planta expression of a polynucleotide encoding a VHH specifically binding to a pathogen.

In certain embodiments of the methods or uses as taught herein, a VHH as taught herein may act as an antimicrobial agent. In certain embodiments of the methods or uses as taught herein, a VHH as taught herein may act as a microbiostatic agent.

The applicants herein have found polynucleotides encoding a VHH specifically binding to a sphingolipid of a fungus, preferably to a ceramide of a fungus, more preferably to a glycosphingolipid of a fungus, even more preferably to a cerebroside of a fungus, yet more preferably to a glucocerebroside of a fungus. Accordingly, a further aspect of the disclosure relates to a transgenic plant or plant tissue or plant cell comprising at least one polynucleotide comprising at least one nucleic acid sequence selected from SEQ ID NO:336 and/or SEQ ID NO:337. In certain embodiments of the transgenic plants or plant tissues or plant cells, methods, or uses as taught herein, the polynucleotide may comprise the nucleic acid sequence of SEQ ID NO:336. In certain embodiments of the transgenic plants or plant tissues or plant cells, methods, or uses as taught herein, the polynucleotide may comprise the nucleic acid sequence of SEQ ID NO:337.

The above and further aspects and preferred embodiments of the disclosure are described in the following sections and in the appended claims. The subject-matter of appended claims is hereby specifically incorporated in this specification.

### BRIEF DESCRIPTION OF THE DRAWINGS

FIG. 1: Binding of VHH as crude VHH-containing periplasmic extracts to coated fungal GlcCer from Pleurotus citrinopileatus. Anti-GlcCer VHH bind to fungal GlcCer, no binding is observed for unrelated VHH.

FIG. 2: Binding specificity of VHH 41D01. Binding of purified VHH 41D01 at 0.1 μg/ml to coated fungal GlcCer from Fusarium oxysporum or Pleurotus citrinopileatus, and non-fungal GlcCer from plant (soy), or mammal (pork). Bars represent average OD 405 nm values, error bars represent standard errors of the mean of n=6. Anti-GlcCer VHH 41D01 specifically binds fungal GlcCer and not plant or mammalian GlcCer.

FIGS. 3A-3C: Binding specificity of VHH. Binding of purified VHH at 1 g/ml to coated fungal GlcCer from Fusarium oxysporum or Pleurotus citrinopileatus, non-fungal GlcCer from plant (soy), and non-fungal mammalian GlcCer (pig). Different anti-GlcCer VHH specifically bind to different fungal GlcCer, but do not bind to plant GlcCer or mammalian GlcCer.

FIG. 4: Real-time measurement of the antibody-antigen interaction between VHH 41D01 and fungal GlcCer. VHH 41D01 binds fungal GlcCer. A slow dissociation of GlcCer from VHH 41D01 is observed. Unrelated VHH_A does not bind fungal GlcCer.

FIG. 5: Cross-reactivity and specificity of VHH 41D01 and VHH 56F11. Binding of purified VHH 41D01 at 0.1 μg/ml and VHH 56F11 at 1 μg/ml to coated fungal lipid extracts, GlcCer from Pleurotus citrinopileatus, and unrelated compounds: apple pectin, citrus pectin, or potato lectin. Bars represent average OD 405 nm values, error bars represent standard errors of the mean of n=2. Anti-GlcCer VHH 41D01 and VHH 56F11 specifically bind each of the fungal lipid extracts tested. Anti-GlcCer VHH 41D01 and VHH 56F11 do not show binding to unrelated coated compounds or non-coated wells.

FIG. 6: Binding of VHH 41D01 in different compositions to fungal GlcCer from Fusarium oxysporum. Aqueous compositions containing anti-GlcCer VHH 41D01 at 0.1 μg/ml and protease inhibitors and/or non-ionic surfactant and/or preservative were tested for binding to fungal GlcCer. GlcCer-specific VHH 41D01 binds to fungal GlcCer in all compositions tested without adverse effects of any of the additives.

FIG. 7: Schematic representation of localization and protein expression of VHH constructs as taught herein. VHH: variable domain of a heavy-chain antibody; His: His6 tag, consists of 6 His repeats (SEQ ID NO:348); KDEL: ER retention signal (SEQ ID NO:349); 2S2: seed storage protein gene signal peptide (SEQ ID NO:350); Fc: Fc from mouse IgG3 (SEQ ID NO:351); linker: 9GS spacer (SEQ ID NO:352); CW: cell wall; PM: plasma membrane; ER: endoplasmatic reticulum; TGN: trans-Golgi network; CCV: Clathrin-coated vesicle.

FIG. 8: Binding of sec_56F11_hinge_Fc_HIS to coated fungal GlcCer. Specific binding to coated wells, but not to uncoated wells, was observed for sec_56F11_hinge_Fc_HIS in leaf extract of overexpressing plants.

FIGS. 9A and 9B: Increased resistance against Botrytis cinerea of plants expressing VHH specifically binding fungal GlcCer. Smaller lesions were observed on the leaves of VHH-overexpressing plants inoculated with B. cinerea spores.

### DETAILED DESCRIPTION

This disclosure will be described with respect to particular embodiments but the disclosure is not limited thereto.

Statements (features) and embodiments of the transgenic plants or plant tissues or plant cells, methods, or uses, as disclosed herein are set forth herein below. Each of the statements and embodiments of the disclosure so defined may be combined with any other statement and/or embodiment unless clearly indicated to the contrary. In particular, any feature or features or statements indicated as being preferred or advantageous may be combined with any other feature or features or statement indicated as being preferred or advantageous. Hereto, this disclosure is, in particular, captured by any one or any combination of one or more of the below numbered aspects and embodiments 1 to 91, with any other statement and/or embodiments.

Numbered statements as disclosed in the present application are:
1. A transgenic plant or plant tissue or plant cell comprising at least one polynucleotide comprising at least one sequence encoding a variable domain of a heavy-chain antibody (VHH) specifically binding to a sphingolipid of a fungus.
2. The transgenic plant or plant tissue or plant cell according to statement 1, wherein the sphingolipid is a ceramide.
3. The transgenic plant or plant tissue or plant cell according to statement 1 or 2, wherein the sphingolipid is a glycosphingolipid.
4. The transgenic plant or plant tissue or plant cell according to any one of statements 1 to 3, wherein the sphingolipid is a cerebroside.
5. The transgenic plant or plant tissue or plant cell according to any one of statements 1 to 4, wherein the sphingolipid comprises a C19 sphingoid base with a C-9 methyl group, and two double bonds (Δ4, Δ8).
6. The transgenic plant or plant tissue or plant cell according to any one of statements 1 to 5, wherein the sphingolipid has, comprises, consists of, or is represented by the following structure:

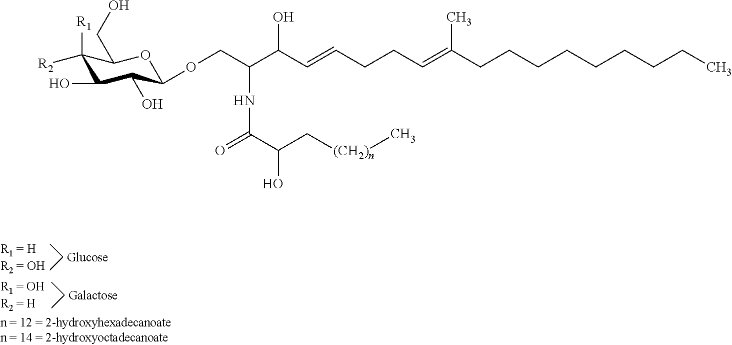

7. The transgenic plant or plant tissue or plant cell according to any one of statements 1 to 6, wherein the sphingolipid has, comprises, consists of, or is represented by any of the following structures:

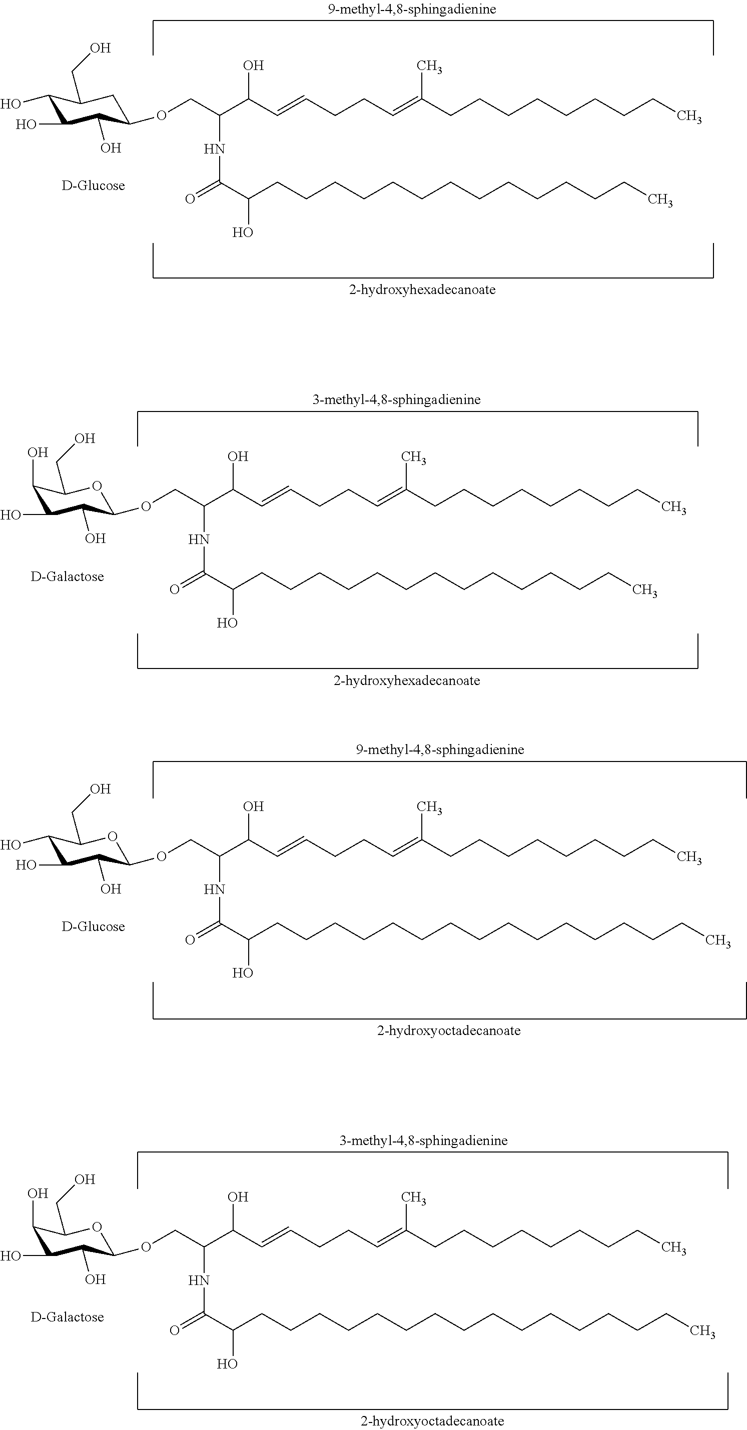

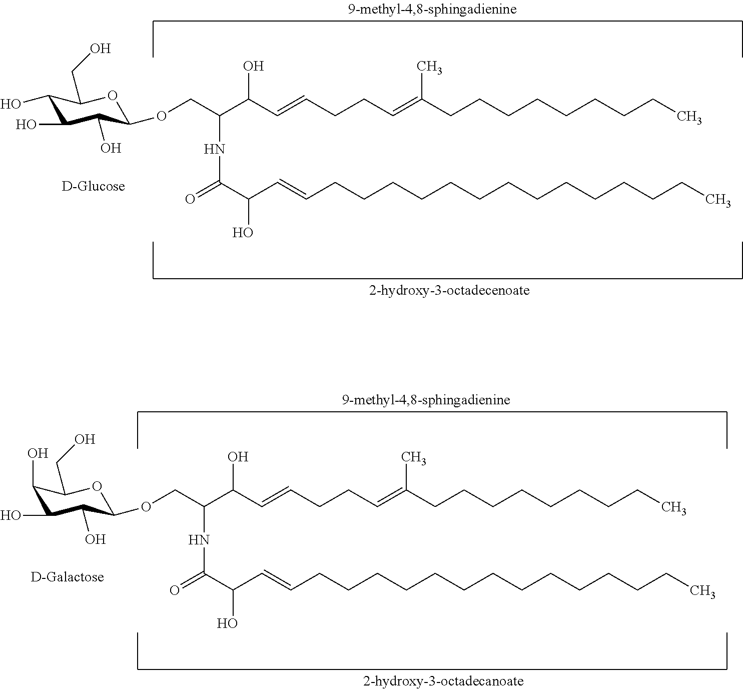

8. The transgenic plant or plant tissue or plant cell according to and one of the statements 1 to 7, wherein the sphingolipid is a glucocerebroside.
9. The transgenic plant or plant tissue or plant cell according to any one of statements 1 to 8, wherein the sphingolipid has, comprises, consists of, or is represented by the following structure:

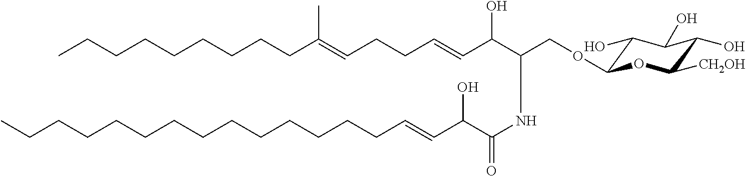

or N-2′-hydroxyhexadecanoyl-1-ß-D-glucopyranosyl-9-methyl-4,8-sphingadienine.
10. The transgenic plant or plant tissue or plant cell according to any one of statements 1 to 9, wherein the expression of the polynucleotide in at least part of the transgenic plant or plant tissue or plant cell (i) protects at least part of the transgenic plant or plant tissue or plant cell from an infection with a plant pathogenic fungus, (ii) inhibits the growth of a plant pathogenic fungus on at least part of the transgenic plant or plant tissue or plant cell, and/or (iii) increases the resistance of at least part of the transgenic plant or plant tissue or plant cell against a plant pathogenic fungus.
11. The transgenic plant or plant tissue or plant cell according to any one of statements 1 to 10, wherein the expression of the polynucleotide in at least part of the transgenic plant or plant tissue or plant cell protects at least part of the transgenic plant or plant tissue or plant cell from an infection with a plant pathogenic fungus.
12. The transgenic plant or plant tissue or plant cell according to any one of statements 1 to 11, wherein the expression of the polynucleotide in at least part of the transgenic plant or plant tissue or plant cell protects at least part of the transgenic plant or plant tissue or plant cell from an infection or other biological interaction with a plant pathogenic fungus.
13. The transgenic plant or plant tissue or plant cell according to any one of statements 1 to 12, wherein the expression of the polynucleotide in at least part of the transgenic plant or plant tissue or plant cell inhibits the growth of a plant pathogenic fungus on at least part of the transgenic plant or plant tissue.
14. The transgenic plant or plant tissue or plant cell according to any one of statements 1 to 13, wherein the expression of the polynucleotide in at least part of the transgenic plant or plant tissue or plant cell increases the resistance of at least part of the transgenic plant or plant tissue or plant cell against a plant pathogenic fungus.
15. The transgenic plant or plant tissue or plant cell according to any one of statements 1 to 14, wherein the expression of the polynucleotide in at least part of the transgenic plant or plant tissue or plant cell (i) protects at least part of the transgenic plant or plant tissue or plant cell from an infection with a plant pathogenic fungus, (ii) inhibits the growth of a plant pathogenic fungus on at least part of the transgenic plant or plant tissue or plant cell, and (iii) increases the resistance of at least part of the transgenic plant or plant tissue or plant cell against a plant pathogenic fungus.
16. The transgenic plant or plant tissue or plant cell according to any one of statements 1 to 15, wherein the polynucleotide comprises a promoter suitable for expression in plants, a plant tissue or plant cell specific promoter, or an inducible promoter.
17. The transgenic plant or plant tissue or plant cell according to any one of statements 1 to 16, wherein the polynucleotide comprises a promoter suitable for expression in plants such as a the 35S Cauliflower Mosaic Virus (CaMV) promoter.
18. The transgenic plant or plant tissue or plant cell according to any one of statements 1 to 17, wherein the polynucleotide comprises at least one sequence encoding a targeting signal for secretion, for location to the cytoplasm, or for location to cellular compartments or organelles, such as the endoplasmatic reticulum (ER) lumen, the apoplast, the vacuole, or intra- and/or exterior membranes.
19. The transgenic plant or plant tissue or plant cell according to any one of statements 1 to 18, wherein the polynucleotide comprises at least one sequence encoding a tag, preferably a His6, c-myc, FLAG, C-tag, 3×FLAG, His5, HA, T7, strep, HSV, and/or an E-tag.
20. The transgenic plant or plant tissue or plant cell according to any one of statements 1 to 19, wherein the polynucleotide encodes the VHH as such, as a combination with one or more identical or different VHHs, or as a combination with one or more identical or different VHHs with a fragment crystallizable region (Fc region); optionally with a spacer.
21. The transgenic plant or plant tissue or plant cell according to any one of statements 1 to 20, wherein the polynucleotide encodes the VHH as such, optionally with a spacer.
22. The transgenic plant or plant tissue or plant cell according to any one of statements 1 to 21, wherein the polynucleotide encodes the VHH as a combination with one or more identical or different VHHs, optionally with a spacer.
23. The transgenic plant or plant tissue or plant cell according to any one of statements 1 to 22, wherein the polynucleotide encodes the VHH as a combination with one or more identical or different VHHs with a fragment crystallizable region (Fc region), optionally with a spacer.
24. The transgenic plant or plant tissue or plant cell according to any one of statements 1 to 23, wherein the plant is a plant selected from the group consisting of corn, rice, wheat, barley, sorghum, millet oats, rye, triticale or other cereals, soybean, alfalfa or other leguminous crops, sugar beet, fodder beet, papaya, banana and plantains or other fruits, grapevines, nuts, oilseed rape, sunflower or other oil crops, squash cucumber, melons or other cucurbits, cotton or other fiber plants, sugarcane, palm, jatropha or other fuel crops, cabbages, tomato, pepper or other vegetables, ornamentals, shrubs, poplar, eucalyptus or other trees, evergreens, grasses, coffee plants, tea plants, tobacco plants, hop plants, rubber plants, and latex plants.
25. The transgenic plant or plant tissue or plant cell according to any one of statements 1 to 24, wherein the plant is selected from the group consisting of banana, barley oat rye, canola, corn, cotton, potato, rice, soybean, tobacco, and wheat, preferably wheat triticale.
26. The transgenic plant or plant tissue or plant cell according to any one of statements 1 to 25, wherein the plant is selected from the group consisting of canola, corn, rice, soybean, and wheat.
27. The transgenic plant or plant tissue or plant cell according to any one of statements 1 to 26, wherein the plant is selected from the group consisting of rice, soybean, and wheat.
28. The transgenic plant or plant tissue or plant cell according to any one of statements 1 to 27, wherein the polynucleotide comprises a sequence encoding a VHH comprising any one or more of SEQ ID NOS:1 to 84.
29. The transgenic plant or plant tissue or plant cell according to any one of statements 1 to 28, wherein the polynucleotide comprises a sequence encoding a VHH comprising SEQ ID NO:1, SEQ ID NO:2, and/or SEQ ID NO:70.
30. The transgenic plant or plant tissue or plant cell according to any one of statements 1 to 29, wherein the polynucleotide comprises a sequence encoding a VHH comprising SEQ ID NOS:1 and/or 2.
31. The transgenic plant or plant tissue or plant cell according to any one of statements 1 to 30, wherein the polynucleotide comprises a sequence encoding a VHH comprising SEQ ID NO:1.
32. The transgenic plant or plant tissue or plant cell according to any one of statements 1 to 31, wherein the polynucleotide comprises a sequence encoding a VHH comprising SEQ ID NO:2.
33. The transgenic plant or plant tissue or plant cell according to any one of statements 1 to 32, wherein the polynucleotide comprises a sequence encoding a VHH comprising SEQ ID NO:70.
34. The transgenic plant or plant tissue or plant cell according to any one of statements 1 to 33, wherein the polynucleotide comprises a sequence encoding a VHH comprising: a CDR1, CDR2, and CDR3 region, wherein (i) the CDR1 region is selected from the group of SEQ ID NOS:85-168, and/or (ii) the CDR2 region is selected from the group of SEQ ID NOS:169-252, and/or (iii) the CDR3 region is selected from the group of SEQ ID NOS:253-335, or the CDR3 region has the amino acid sequence NRY.
35. The transgenic plant or plant tissue or plant cell according to any one of statements 1 to 34, wherein the polynucleotide comprises a sequence encoding a VHH comprising a CDR1, CDR2 and CDR3 region chosen from the list of comprising:
   a CDR1 region having SEQ ID NO:85, a CDR2 region having has SEQ ID NO:169, and a CDR3 region having SEQ ID NO:253, and/or
   a CDR1 region having SEQ ID NO:86, a CDR2 region having has SEQ ID NO:170, and a CDR3 region having SEQ ID NO:254, and/or
   a CDR1 region having SEQ ID NO:87, a CDR2 region having has SEQ ID NO:171, and a CDR3 region having SEQ ID NO:255, and/or
   a CDR1 region having SEQ ID NO:88, a CDR2 region having has SEQ ID NO:172, and a CDR3 region having SEQ ID NO:256, and/or
   a CDR1 region having SEQ ID NO:89, a CDR2 region having has SEQ ID NO:173, and a CDR3 region having SEQ ID NO:257, and/or
   a CDR1 region having SEQ ID NO:90, a CDR2 region having has SEQ ID NO:174, and a CDR3 region having SEQ ID NO:258, and/or
   a CDR1 region having SEQ ID NO:91, a CDR2 region having has SEQ ID NO:175, and a CDR3 region having SEQ ID NO:259, and/or
   a CDR1 region having SEQ ID NO:92, a CDR2 region having has SEQ ID NO:176, and a CDR3 region having SEQ ID NO:260, and/or
   a CDR1 region having SEQ ID NO:93, a CDR2 region having has SEQ ID NO:177, and a CDR3 region having SEQ ID NO:261, and/or
   a CDR1 region having SEQ ID NO:94, a CDR2 region having has SEQ ID NO:178, and a CDR3 region having SEQ ID NO:262, and/or
   a CDR1 region having SEQ ID NO:95, a CDR2 region having has SEQ ID NO:179, and a CDR3 region having SEQ ID NO:263, and/or
   a CDR1 region having SEQ ID NO:96, a CDR2 region having has SEQ ID NO:180, and a CDR3 region having SEQ ID NO:264, and/or
   a CDR1 region having SEQ ID NO:97, a CDR2 region having has SEQ ID NO:181, and a CDR3 region having SEQ ID NO:265, and/or
   a CDR1 region having SEQ ID NO:98, a CDR2 region having has SEQ ID NO:182, and a CDR3 region having SEQ ID NO:266, and/or
   a CDR1 region having SEQ ID NO:99, a CDR2 region having has SEQ ID NO:183, and a CDR3 region having SEQ ID NO:267, and/or
   a CDR1 region having SEQ ID NO:100, a CDR2 region having has SEQ ID NO:184, and a CDR3 region having SEQ ID NO:268, and/or
   a CDR1 region having SEQ ID NO:101, a CDR2 region having has SEQ ID NO:185, and a CDR3 region having SEQ ID NO:269, and/or
   a CDR1 region having SEQ ID NO:102, a CDR2 region having has SEQ ID NO:186, and a CDR3 region having SEQ ID NO:270, and/or
   a CDR1 region having SEQ ID NO:103, a CDR2 region having has SEQ ID NO:187, and a CDR3 region having SEQ ID NO:271, and/or
   a CDR1 region having SEQ ID NO:104, a CDR2 region having has SEQ ID NO:188, and a CDR3 region having SEQ ID NO:272, and/or
   a CDR1 region having SEQ ID NO:105, a CDR2 region having has SEQ ID NO:189, and a CDR3 region having SEQ ID NO:273, and/or
   a CDR1 region having SEQ ID NO:106, a CDR2 region having has SEQ ID NO:190, and a CDR3 region having SEQ ID NO:274, and/or
   a CDR1 region having SEQ ID NO:107, a CDR2 region having has SEQ ID NO:191, and a CDR3 region having SEQ ID NO:275, and/or
   a CDR1 region having SEQ ID NO:108, a CDR2 region having has SEQ ID NO:192, and a CDR3 region having SEQ ID NO:276, and/or
   a CDR1 region having SEQ ID NO:109, a CDR2 region having has SEQ ID NO:193, and a CDR3 region having SEQ ID NO:277, and/or
   a CDR1 region having SEQ ID NO:110, a CDR2 region having has SEQ ID NO:194, and a CDR3 region having SEQ ID NO:278, and/or
   a CDR1 region having SEQ ID NO:111, a CDR2 region having has SEQ ID NO:195, and a CDR3 region having SEQ ID NO:279, and/or
   a CDR1 region having SEQ ID NO:112, a CDR2 region having has SEQ ID NO:196, and a CDR3 region having SEQ ID NO:280, and/or
   a CDR1 region having SEQ ID NO:113, a CDR2 region having has SEQ ID NO:197, and a CDR3 region having SEQ ID NO:281, and/or
   a CDR1 region having SEQ ID NO:114, a CDR2 region having has SEQ ID NO:198, and a CDR3 region having SEQ ID NO:282, and/or
   a CDR1 region having SEQ ID NO:115, a CDR2 region having has SEQ ID NO:199, and a CDR3 region having SEQ ID NO:283, and/or
   a CDR1 region having SEQ ID NO:116, a CDR2 region having has SEQ ID NO:200, and a CDR3 region having SEQ ID NO:284, and/or
   a CDR1 region having SEQ ID NO:117, a CDR2 region having has SEQ ID NO:201, and a CDR3 region having SEQ ID NO:285, and/or
   a CDR1 region having SEQ ID NO:118, a CDR2 region having has SEQ ID NO:202, and a CDR3 region having SEQ ID NO:286, and/or
   a CDR1 region having SEQ ID NO:119, a CDR2 region having has SEQ ID NO:203, and a CDR3 region having SEQ ID NO:287, and/or
   a CDR1 region having SEQ ID NO:120, a CDR2 region having has SEQ ID NO:204, and a CDR3 region having SEQ ID NO:288, and/or
   a CDR1 region having SEQ ID NO:121, a CDR2 region having has SEQ ID NO:205, and a CDR3 region having SEQ ID NO:289, and/or
   a CDR1 region having SEQ ID NO:122, a CDR2 region having has SEQ ID NO:206, and a CDR3 region having SEQ ID NO:290, and/or
   a CDR1 region having SEQ ID NO:123, a CDR2 region having has SEQ ID NO:207, and a CDR3 region having SEQ ID NO:291, and/or
   a CDR1 region having SEQ ID NO:124, a CDR2 region having has SEQ ID NO:208, and a CDR3 region having SEQ ID NO:292, and/or
   a CDR1 region having SEQ ID NO:125, a CDR2 region having has SEQ ID NO:209, and a CDR3 region having SEQ ID NO:293, and/or
   a CDR1 region having SEQ ID NO:126, a CDR2 region having has SEQ ID NO:210, and a CDR3 region having SEQ ID NO:294, and/or
   a CDR1 region having SEQ ID NO:127, a CDR2 region having has SEQ ID NO:211, and a CDR3 region having SEQ ID NO:295, and/or
   a CDR1 region having SEQ ID NO:128, a CDR2 region having has SEQ ID NO:212, and a CDR3 region having SEQ ID NO:296, and/or
   a CDR1 region having SEQ ID NO:129, a CDR2 region having has SEQ ID NO:213, and a CDR3 region having SEQ ID NO:297, and/or
   a CDR1 region having SEQ ID NO:130, a CDR2 region having has SEQ ID NO:214, and a CDR3 region having SEQ ID NO:298, and/or
   a CDR1 region having SEQ ID NO:131, a CDR2 region having has SEQ ID NO:215, and a CDR3 region having SEQ ID NO:299, and/or
   a CDR1 region having SEQ ID NO:132, a CDR2 region having has SEQ ID NO:216, and a CDR3 region having SEQ ID NO:300, and/or
   a CDR1 region having SEQ ID NO:133, a CDR2 region having has SEQ ID NO:217, and a CDR3 region having SEQ ID NO:301, and/or
   a CDR1 region having SEQ ID NO:134, a CDR2 region having has SEQ ID NO:218, and a CDR3 region having SEQ ID NO:302, and/or
   a CDR1 region having SEQ ID NO:135, a CDR2 region having has SEQ ID NO:219, and a CDR3 region having SEQ ID NO:303, and/or
   a CDR1 region having SEQ ID NO:136, a CDR2 region having has SEQ ID NO:220, and a CDR3 region having SEQ ID NO:304, and/or
   a CDR1 region having SEQ ID NO:137, a CDR2 region having has SEQ ID NO:221, and a CDR3 region having SEQ ID NO:305, and/or
   a CDR1 region having SEQ ID NO:138, a CDR2 region having has SEQ ID NO:222, and a CDR3 region having SEQ ID NO:306, and/or
   a CDR1 region having SEQ ID NO:139, a CDR2 region having has SEQ ID NO:223, and a CDR3 region having the amino acid sequence NRY, and/or
   a CDR1 region having SEQ ID NO:140, a CDR2 region having has SEQ ID NO:224, and a CDR3 region having SEQ ID NO:307, and/or
   a CDR1 region having SEQ ID NO:141, a CDR2 region having has SEQ ID NO:225, and a CDR3 region having SEQ ID NO:308, and/or
   a CDR1 region having SEQ ID NO:142, a CDR2 region having has SEQ ID NO:226, and a CDR3 region having SEQ ID NO:309, and/or
   a CDR1 region having SEQ ID NO:143, a CDR2 region having has SEQ ID NO:227, and a CDR3 region having SEQ ID NO:310, and/or
   a CDR1 region having SEQ ID NO:144, a CDR2 region having has SEQ ID NO:228, and a CDR3 region having SEQ ID NO:311, and/or
   a CDR1 region having SEQ ID NO:145, a CDR2 region having has SEQ ID NO:229, and a CDR3 region having SEQ ID NO:312, and/or
   a CDR1 region having SEQ ID NO:146, a CDR2 region having has SEQ ID NO:230, and a CDR3 region having SEQ ID NO:313, and/or
   a CDR1 region having SEQ ID NO:147, a CDR2 region having has SEQ ID NO:231, and a CDR3 region having SEQ ID NO:314, and/or
   a CDR1 region having SEQ ID NO:148, a CDR2 region having has SEQ ID NO:232, and a CDR3 region having SEQ ID NO:315, and/or
   a CDR1 region having SEQ ID NO:149, a CDR2 region having has SEQ ID NO:233, and a CDR3 region having SEQ ID NO:316, and/or
   a CDR1 region having SEQ ID NO:150, a CDR2 region having has SEQ ID NO:234, and a CDR3 region having SEQ ID NO:317, and/or
   a CDR1 region having SEQ ID NO:151, a CDR2 region having has SEQ ID NO:235, and a CDR3 region having SEQ ID NO:318, and/or
   a CDR1 region having SEQ ID NO:152, a CDR2 region having has SEQ ID NO:236, and a CDR3 region having SEQ ID NO:319, and/or
   a CDR1 region having SEQ ID NO:153, a CDR2 region having has SEQ ID NO:237, and a CDR3 region having SEQ ID NO:320, and/or
   a CDR1 region having SEQ ID NO:154, a CDR2 region having has SEQ ID NO:238, and a CDR3 region having SEQ ID NO:321, and/or
   a CDR1 region having SEQ ID NO:155, a CDR2 region having has SEQ ID NO:239, and a CDR3 region having SEQ ID NO:322, and/or
   a CDR1 region having SEQ ID NO:156, a CDR2 region having has SEQ ID NO:240, and a CDR3 region having SEQ ID NO:323, and/or
   a CDR1 region having SEQ ID NO:157, a CDR2 region having has SEQ ID NO:241, and a CDR3 region having SEQ ID NO:324, and/or
   a CDR1 region having SEQ ID NO:158, a CDR2 region having has SEQ ID NO:242, and a CDR3 region having SEQ ID NO:325, and/or
   a CDR1 region having SEQ ID NO:159, a CDR2 region having has SEQ ID NO:243, and a CDR3 region having SEQ ID NO:326, and/or
   a CDR1 region having SEQ ID NO:160, a CDR2 region having has SEQ ID NO:244, and a CDR3 region having SEQ ID NO:327, and/or
   a CDR1 region having SEQ ID NO:161, a CDR2 region having has SEQ ID NO:245, and a CDR3 region having SEQ ID NO:328, and/or
   a CDR1 region having SEQ ID NO:162, a CDR2 region having has SEQ ID NO:246, and a CDR3 region having SEQ ID NO:329, and/or
   a CDR1 region having SEQ ID NO:163, a CDR2 region having has SEQ ID NO:247, and a CDR3 region having SEQ ID NO:330, and/or
   a CDR1 region having SEQ ID NO:164, a CDR2 region having has SEQ ID NO:248, and a CDR3 region having SEQ ID NO:331, and/or
   a CDR1 region having SEQ ID NO:165, a CDR2 region having has SEQ ID NO:249, and a CDR3 region having SEQ ID NO:332, and/or
   a CDR1 region having SEQ ID NO:166, a CDR2 region having has SEQ ID NO:250, and a CDR3 region having SEQ ID NO:333, and/or
   a CDR1 region having SEQ ID NO:167, a CDR2 region having has SEQ ID NO:251, and a CDR3 region having SEQ ID NO:334, and/or
   a CDR1 region having SEQ ID NO:168, a CDR2 region having has SEQ ID NO:252, and a CDR3 region having SEQ ID NO:335.
36. The transgenic plant or plant tissue or plant cell according to any one of statements 1 to 35, wherein the polynucleotide comprises a sequence encoding a VHH comprising a CDR1 region having SEQ ID NO:85, a CDR2 region having has SEQ ID NO:169, and a CDR3 region having SEQ ID NO:253.
37. The transgenic plant or plant tissue or plant cell according to any one of statements 1 to 35, wherein the polynucleotide comprises a sequence encoding a VHH comprising a CDR1 region having SEQ ID NO:86, a CDR2 region having has SEQ ID NO:170, and a CDR3 region having SEQ ID NO:254.
38. Harvestable parts and propagation materials of a transgenic plant or plant tissue or plant cell according to any one of statements 1 to 37, comprising at least one polynucleotide as defined in any one of statements 1 to 37.
39. The harvestable parts and propagation materials according to statement 38, wherein the harvestable parts and propagation materials of a transgenic plant or plant tissue or plant cell are selected from the group consisting of seeds, fruits, grains, bulbs, bolls, tubers, progeny, and hybrids.
40. A method for the production of a transgenic plant or plant tissue or plant cell comprising the introduction of at least one polynucleotide as defined in any one of statements 1 to 37 into the genome of a plant or plant tissue.
41. The use of at least one polynucleotide as defined in any one of statements 1 to 37, for the production of a transgenic plant or plant tissue.
42. A method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising expressing in at least part of the plant or plant tissue or plant cell at least one polynucleotide encoding a variable domain of a heavy-chain antibody (VHH) specifically binding to a pathogen.
43. The method according to statement 42, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, comprising expressing in at least part of the plant or plant tissue or plant cell at least one polynucleotide as defined any one of statements 1 to 37.
44. The method according to statement 42 or 43, wherein the VHH specifically binds to a sphingolipid of a pathogen.
45. The method according to any one of statements 42 to 44, wherein the VHH specifically binds to a ceramide of a pathogen.
46. The method according to any one of statements 44 to 45, wherein the VHH specifically binds to a glycosphingolipid of a pathogen.
47. The method according to any one of statements 44 to 46, wherein the VHH specifically binds to a cerebroside of a pathogen.
48. The method according to any one of statements 44 to 47, wherein the VHH specifically binds to a glucocerebroside of a pathogen.
49. The method according to any one of statements 44 to 48, wherein the VHH binds to a plant pathogen.
50. The method according to any one of statements 44 to 49, wherein the VHH binds to a sphingolipid of a plant pathogen.
51. The method according to any one of statements 44 to 50, wherein the VHH binds to a ceramide of a plant pathogen.
52. The method according to any one of statements 44 to 51, wherein the VHH binds to a glycosphingolipid of a plant pathogen.
53. The method according to any one of statements 44 to 52, wherein the VHH binds to a cerebroside of a plant pathogen.
54. The method according to any one of statements 44 to 53, wherein the VHH binds to a glucocerebroside of a plant pathogenic fungus.
55. The method according to statements 44 to 54, wherein the VHH specifically binds to a fungus.
56. The method according to statements 44 to 55, wherein the VHH specifically binds to a sphingolipid of a fungus.
57. The method according to any one of statements 44 to 56, wherein the VHH specifically binds to a ceramide of a fungus.
58. The method according to any one of statements 44 to 57, wherein the VHH specifically binds to a glycosphingolipid of a fungus.
59. The method according to any one of statements 44 to 58, wherein the VHH specifically binds to a cerebroside of a fungus.
60. The method according to any one of statements 44 to 59, wherein the VHH specifically binds to a glucocerebroside of a fungus.
61. The method according to any one of statements 44 to 60, wherein the VHH binds to a plant pathogenic fungus.
62. The method according to any one of statements 44 to 61, wherein the VHH binds to a sphingolipid of a plant pathogenic fungus.
63. The method according to any one of statements 44 to 62, wherein the VHH binds to a ceramide of a plant pathogenic fungus.
64. The method according to any one of statements 44 to 63, wherein the VHH binds to a glycosphingolipid of a plant pathogenic fungus.
65. The method according to any one of statements 44 to 64, wherein the VHH binds to a cerebroside of a plant pathogenic fungus.
66. The method according to any one of statements 44 to 65, wherein the VHH binds to a glucocerebroside of a plant pathogenic fungus.
67. Use of at least one polynucleotide encoding a variable domain of a heavy-chain antibody (VHH) specifically binding to a pathogen, for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, wherein the polynucleotide is expressed in at least part of the plant or plant tissue.
68. The use according to statement 67, for protecting at least part of a plant or plant tissue or plant cell from an infection with a plant pathogenic fungus, for inhibiting the growth of a plant pathogenic fungus on at least part of a plant or plant tissue or plant cell, and/or for increasing pathogen resistance of at least part of a plant or plant tissue or plant cell, wherein the polynucleotide is defined as in any one of statements 1 to 37.
69. The use according to statement 67 or 64, wherein the VHH specifically binds to a sphingolipid of a pathogen.
70. The use according to any one of statements 67 to 69, wherein the VHH specifically binds to a ceramide of a pathogen.
71. The use according to any one of statements 67 to 70, wherein the VHH specifically binds to a glycosphingolipid of a pathogen.
72. The use according to any one of statements 67 to 71, wherein the VHH specifically binds to a cerebroside of a pathogen.
73. The use according to any one of statements 67 to 72, wherein the VHH specifically binds to a glucocerebroside of a pathogen.
74. The use according to any one of statements 67 to 73, wherein the VHH binds to a plant pathogen.
75. The use according to any one of statements 67 to 74, wherein the VHH binds to a sphingolipid of a plant pathogen.
76. The use according to any one of statements 67 to 75, wherein the VHH binds to a ceramide of a plant pathogen.
77. The use according to any one of statements 67 to 76, wherein the VHH binds to a glycosphingolipid of a plant pathogen.
78. The use according to any one of statements 67 to 77, wherein the VHH binds to a cerebroside of a plant pathogen.
79. The use according to any one of statements 67 to 78, wherein the VHH binds to a glucocerebroside of a plant pathogenic fungus.
80. The use according to statements 67 to 79, wherein the VHH specifically binds to a fungus.
81. The use according to statements 67 to 80, wherein the VHH specifically binds to a sphingolipid of a fungus.
82. The use according to any one of statements 67 to 81, wherein the VHH specifically binds to a ceramide of a fungus.
83. The use according to any one of statements 67 to 82, wherein the VHH specifically binds to a glycosphingolipid of a fungus.
84. The use according to any one of statements 67 to 83, wherein the VHH specifically binds to a cerebroside of a fungus.
85. The use according to any one of statements 67 to 84, wherein the VHH specifically binds to a glucocerebroside of a fungus.
86. The use according to any one of statements 67 to 85, wherein the VHH binds to a plant pathogenic fungus.
87. The use according to any one of statements 67 to 86, wherein the VHH binds to a sphingolipid of a plant pathogenic fungus.
88. The use according to any one of statements 67 to 87, wherein the VHH binds to a ceramide of a plant pathogenic fungus.
89. The use according to any one of statements 67 to 88, wherein the VHH binds to a glycosphingolipid of a plant pathogenic fungus.
90. The use according to any one of statements 67 to 89, wherein the VHH binds to a cerebroside of a plant pathogenic fungus.
91. The use according to any one of statements 67 to 90, wherein the VHH binds to a glucocerebroside of a plant pathogenic fungus.
92. An extract of a transgenic plant or plant tissue or plant cell according to any one of statements 1 to 37, the extract comprising VHH.
93. A composition comprising the extract of statement 92.
94. A method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising treating at least part of a plant or plant tissue or plant cell with the extract of statement 92 or the composition of statement 93.
95. Use of the extract of statement 92 or the composition of claim 93 for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell.

### Definitions

This disclosure will be described with respect to particular embodiments but the disclosure is not limited thereto but only by the claims. Any reference signs in the claims shall not be construed as limiting the scope.

Where the term “comprising” is used in the present description and claims, it does not exclude other elements or steps.

Where an indefinite or definite article is used when referring to a singular noun, e.g., “a,” “an,” or “the,” this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The following terms or definitions are provided solely to aid in the understanding of the disclosure. Unless specifically defined herein, all terms used herein have the same meaning as they would to one skilled in the art of this disclosure. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is, for example, again made to the standard handbooks, to the general background art referred to above and to the further references cited therein.

As used herein, the terms “polypeptide,” “protein,” “peptide,” and “amino acid sequence” are used interchangeably, and refer to a polymeric form of amino acids of any length, which can include coded and non-coded amino acids, chemically or biochemically modified or derivatized amino acids, and polypeptides having modified peptide backbones.

As used herein, amino acid residues will be indicated either by their full name or according to the standard three-letter or one-letter amino acid code.

As used herein, the terms “nucleic acid molecule,” “polynucleotide,” “polynucleic acid,” and “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural similarity between two macromolecules, particularly between two polypeptides or polynucleotides, from same or different taxons, wherein the similarity is due to shared ancestry. Hence, the term “homologues” denotes so-related macromolecules having secondary and optionally tertiary structural similarity.

For comparing two or more polynucleotide sequences, the “(percentage of) sequence identity” between a first polynucleotide sequence and a second polynucleotide sequence may be calculated using methods known by the person skilled in the art, e.g., by optimally aligning the polynucleotide sequences and introducing gaps, if necessary, followed by dividing the number of nucleotides in the first polynucleotide sequence that are identical to the nucleotides at the corresponding positions in the second polynucleotide sequence in a comparison window by the number of positions in the comparison window (the window size), and multiplying by 100%. Optimal sequence alignment of two or more polynucleotide sequences over a comparison window can be obtained by using a known computer algorithm for sequence alignment such as NCBI Blast (Altschul et al., J. Mol. Biol. 1990 Oct. 5; 215 (3):403-410). Another example of an algorithm that is suitable for polynucleotide sequence alignments is the CLUSTALW program (J. D. Thompson, et al., Nucl. Acids Res. 1994 Nov. 11; 22 (22):4673-4680). CLUSTALW performs multiple pairwise comparisons between groups of sequences and assembles them into a multiple alignment based on homology. For comparing two or more polypeptide sequences, the “(percentage of) sequence identity” between a first polypeptide sequence and a second polypeptide sequence may be calculated using methods known by the person skilled in the art, e.g., by optimally aligning the polypeptide sequences and introducing gaps, if necessary, followed by dividing the number of amino acids in the first polypeptide sequence that are identical to the amino acids at the corresponding positions in the second polypeptide sequence in a comparison window by the number of positions in the comparison window (the window size), and multiplying by 100%. Optimal sequence alignment of two or more polypeptide sequences over a comparison window can be obtained by using a known computer algorithm for sequence alignment such as NCBI Blast (Altschul et al., J. Mol. Biol. 1990 Oct. 5; 215 (3):403-410). Another example of an algorithm that is suitable for polypeptide sequence alignments is the CLUSTALW program (J. D. Thompson et al., Nucl. Acids Res. 1994 Nov. 11; 22 (22):4673-4680). CLUSTALW performs multiple pairwise comparisons between groups of sequences and assembles them into a multiple alignment based on homology. For amino acid alignments, the BLOSUM algorithm can be used as a protein weight matrix (Henikoff and Henikoff, PNAS USA 1992 Nov. 15; 89 (22):10915-10919). In determining the degree of sequence homology between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Conservative amino acid substitutions are counted as identities in order to calculate the percentage homology between two polypeptide sequences. Possible conservative amino acid substitutions will be clear to the person skilled in the art.

As used herein, “comparison window” makes reference to a contiguous and specified segment of an optimal alignment of polynucleotide or polypeptide sequences, wherein the sequences in the comparison window may comprise gaps for optimal alignment of the two sequences. The comparison window for determining sequence identity or homology may be as long as the longest of the aligned sequences, or as long as the shortest of the aligned sequences, or as long as the alignment including gaps in any of the sequences introduced to optimize the alignment. Comparison windows may be about 5000 positions long, or about 2000 positions, or about 1000 positions, or about 800 positions, or about 600 positions long, or about 500 positions long, or about 400 positions long, or about 300 positions long, or about 200 positions long, or about 100 positions long, or about 50 positions long, or about 40 positions long, or about 30 positions long, or about 20 positions long, or about 10 positions long. Amino acid sequences and nucleic acid sequences are said to be “exactly the same” if they have 100% sequence identity over their entire length.

As used herein, the terms “complementarity-determining region” or “CDR” within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have three CDR regions, each non-contiguous with the others (termed L1, L2, L3, H1, H2, H3) for the respective light (L) and heavy (H) chains.

The term “affinity,” as used herein, refers to the degree to which a polypeptide, in particular, an immunoglobulin, such as an antibody, or an immunoglobulin fragment, such as a VHH, binds to an antigen so as to shift the equilibrium of antigen and polypeptide toward the presence of a complex formed by their binding. Thus, for example, where an antigen and antibody (fragment) are combined in relatively equal concentration, an antibody (fragment) of high affinity will bind to the available antigen so as to shift the equilibrium toward high concentration of the resulting complex. The dissociation constant is commonly used to describe the affinity between the protein binding domain and the antigenic target. Typically, the dissociation constant is lower than 10⁻⁵ M. Preferably, the dissociation constant is lower than 10⁻⁶ M, and more preferably, lower than 10⁻⁷ M. Most preferably, the dissociation constant is lower than 10⁻⁸ M.

The terms “specifically bind” and “specific binding,” as used herein, generally refers to the ability of a polypeptide, in particular, an immunoglobulin, such as an antibody, or an immunoglobulin fragment, such as a VHH, to preferentially bind to a particular antigen that is present in a homogeneous mixture of different antigens. In certain embodiments, a specific binding interaction will discriminate between desirable and undesirable antigens in a sample, in some embodiments more than about 10- to 100-fold or more (e.g., more than about 1000- or 10,000-fold).

Accordingly, an amino acid sequence as disclosed herein is said to “specifically bind to” a particular target when that amino acid sequence has affinity for, specificity for and/or is specifically directed against that target (or for at least one part or fragment thereof).

The “specificity” of an amino acid sequence as disclosed herein can be determined based on affinity and/or avidity.

An amino acid sequence as disclosed herein is said to be “specific for a first target antigen of interest as opposed to a second target antigen of interest” when it binds to the first target antigen of interest with an affinity that is at least 5 times, such as at least 10 times, such as at least 100 times, and preferably at least 1000 times higher than the affinity with which that amino acid sequence as disclosed herein binds to the second target antigen of interest. Accordingly, in certain embodiments, when an amino acid sequence as disclosed herein is said to be “specific for” a first target antigen of interest as opposed to a second target antigen of interest, it may specifically bind to (as defined herein) the first target antigen of interest, but not to the second target antigen of interest.

As used herein, the terms “inhibiting,” “reducing” and/or “preventing” may refer to (the use of) an amino acid sequence as disclosed herein that specifically binds to a target antigen of interest and inhibits, reduces and/or prevents the interaction between that target antigen of interest, and its natural binding partner. The terms “inhibiting,” “reducing” and/or “preventing” may also refer to (the use of) an amino acid sequence as disclosed herein that specifically binds to a target antigen of interest and inhibits, reduces and/or prevents a biological activity of that target antigen of interest, as measured using a suitable in vitro, cellular or in vivo assay. Accordingly, “inhibiting,” “reducing” and/or “preventing” may also refer to (the use of) an amino acid sequence as disclosed herein that specifically binds to a target antigen of interest and inhibits, reduces and/or prevents one or more biological or physiological mechanisms, effects, responses, functions pathways or activities in which the target antigen of interest is involved. Such an action of the amino acid sequence as disclosed herein as an antagonist may be determined in any suitable manner and/or using any suitable (in vitro and usually cellular or in vivo) assay known in the art, depending on the target antigen of interest.

Thus, more particularly, “inhibiting,” “reducing” and/or “preventing” using amino acid sequence as disclosed herein may mean either inhibiting, reducing and/or preventing the interaction between a target antigen of interest and its natural binding partner, or inhibiting, reducing and/or preventing the activity of a target antigen of interest, or inhibiting, reducing and/or preventing one or more biological or physiological mechanisms, effects, responses, functions pathways or activities in which the target antigen of interest is involved, such as by at least 10%, but preferably at least 20%, for example, by at least 50%, at least 60%, at least 70%, at least 80%, at least 90%, at least 95% or more, as measured using a suitable in vitro, cellular, or in vivo assay, compared to the activity of the target antigen of interest in the same assay under the same conditions but without using the amino acid sequence as disclosed herein. In addition, “inhibiting,” “reducing” and/or “preventing” may also mean inducing a decrease in affinity, avidity, specificity and/or selectivity of a target antigen of interest for one or more of its natural binding partners and/or inducing a decrease in the sensitivity of the target antigen of interest for one or more conditions in the medium or surroundings in which the target antigen of interest is present (such as pH, ion strength, the presence of co-factors, etc.), compared to the same conditions but without the presence of the amino acid sequence as disclosed herein. In the context of this disclosure, “inhibiting,” “reducing” and/or “preventing” may also involve allosteric inhibition, reduction and/or prevention of the activity of a target antigen of interest.

The inhibiting or antagonizing activity or the enhancing or agonizing activity of an amino acid sequence as disclosed herein may be reversible or irreversible.

In respect of the amino acid sequences as disclosed herein, the terms “binding region,” “binding site” or “interaction site” present on the amino acid sequences as disclosed herein shall herein have the meaning of a particular site, region, locus, part, or domain present on the target molecule, which particular site, region, locus, part, or domain is responsible for binding to that target molecule. Such binding region thus essentially consists of that particular site, region, locus, part, or domain of the target molecule, which is in contact with the amino acid sequence when bound to that target molecule.

“Plant” as used herein, means live plants and live plant parts, including fresh fruit, vegetables and seeds. Also, the term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest.

The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The choice of suitable control plants is a routine part of an experimental setup and may include corresponding wild-type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

The term “plant tissue” as used herein refers to a group of similar plant cells from the same origin that together carry out a specific function. Examples of plant tissues include meristematic tissue, protective tissue, parenchyma, sclerenchyma, collenchyma, xylem, and phloem.

“Crop” as used herein means a plant species or variety that is grown to be harvested as food, livestock fodder, fuel raw material, or for any other economic purpose. As a non-limiting example, the crops can be maize, cereals, such as wheat, rye, barley and oats, sorghum, rice, sugar beet and fodder beet, fruit, such as pome fruit (e.g., apples and pears), citrus fruit (e.g., oranges, lemons, limes, grapefruit, or mandarins), stone fruit (e.g., peaches, nectarines or plums), nuts (e.g., almonds or walnuts), soft fruit (e.g., cherries, strawberries, blackberries or raspberries), the plantain family or grapevines, leguminous crops, such as beans, lentils, peas and soya, oil crops, such as sunflower, safflower, rapeseed, canola, castor or olives, cucurbits, such as cucumbers, melons or pumpkins, fiber plants, such as cotton, flax or hemp, fuel crops, such as sugarcane, miscanthus or switchgrass, vegetables, such as potatoes, tomatoes, peppers, lettuce, spinach, onions, carrots, egg-plants, asparagus or cabbage, ornamentals, such as flowers (e.g., petunias, pelargoniums, roses, tulips, lilies, or chrysanthemums), shrubs, broad-leaved trees (e.g., poplars or willows) and evergreens (e.g., conifers), grasses, such as lawn, turf or forage grass or other useful plants, such as coffee, tea, tobacco, hops, pepper, rubber or latex plants.

A “plant pest,” “plant pathogen” or “crop pest,” as used herein interchangeably, refers to organisms that specifically cause damage to plants, plant parts or plant products, particularly plants, plant parts or plant products, used in agriculture. Note that the term “plant pest” or “crop pest” is used in the meaning that the pest targets and harms plants. Relevant crop pest examples include, but are not limited to pathogenic fungi (including Ascomycetes (such as Fusarium spp., Thielaviopsis spp., Verticillium spp., and Magnaporthe spp.), Basidiomycetes (such as Rhizoctonia spp., Phakospora spp., and Puccinia spp.), and fungal-like Oomycetes (such as Pythium spp. and Phytophthora spp.), aphids, caterpillars, flies, wasps, and the like, nematodes (living freely in soil or particularly species that parasitize plant roots, such as root-knot nematode and cyst nematodes such as soybean cyst nematode and potato cyst nematode), mites (such as spider mites, thread-footed mites and gall mites) and gastropods (including slugs such as Deroceras spp., Milax spp., Tandonia sp., Limax spp., Arion spp. and Veronicella spp. and snails such as Helix spp., Cernuella spp., Theba spp., Cochlicella spp., Achatina spp., Succinea spp., Ovachlamys spp., Amphibulima spp., Zachrysia spp., Bradybaena spp., and Pomacea spp.), bacteria (such as Burkholderia spp. and Proteobacteria such as Xanthomonas spp. and Pseudomonas spp.), Phytoplasma, Spiroplasma, viruses (such as tobacco mosaic virus and cauliflower mosaic virus), and protozoa.

“Microbe,” as used herein, means fungus, yeast, bacterium, virus, and the like and “microbial” means derived from a microbe.

“Fungus,” as used herein, means a eukaryotic organism, belonging to the group of Eumycota. The term “fungus” in this disclosure also includes fungal-like organisms such as the Oomycota. Oomycota (or oomycetes) form a distinct phylogenetic lineage of fungus-like eukaryotic microorganisms. This group was originally classified among the fungi but modern insights support a relatively close relationship with the photosynthetic organisms such as brown algae and diatoms, within the group of heterokonts.

The terms “plant pathogenic fungus” or “fungal plant pathogen,” as used herein interchangeably, refer to any fungus as defined herein capable of causing an infection or other biological reaction on a plant.

The term “infection” as used herein refers to any inflammatory condition, disease or disorder in a plant that is caused by a plant pathogen.

“Fungal infection” or “fungal disease” as used herein refers to any inflammatory condition, disease or disorder in a living organism, such as a plant, animal or human, which is caused by a fungus.

The term “antimicrobial agent,” as used herein, refers to an agent that kills microorganisms or inhibits the growth of microorganisms. Antimicrobial agents encompass antibacterial, antifungal, antiviral, or antiparasitic agents.

The term “microbicidal agent,” as used herein, refers to an agent that kills microorganisms.

The term “microbiostatic agent,” as used herein, refers to an agent that inhibits the growth of microorganisms.

The term “fungicidal agent,” as used herein, refers to an agent that kills a fungus.

The term “fungistatic agent,” as used herein, refers to an agent that inhibits the growth of a fungus.

“Antimicrobial (effect)” or “antimicrobial use,” as used herein, includes any effect or use of an agent, in particular, of a VHH, for controlling, modulating or interfering with the harmful activity of a plant pathogen, including killing the plant pathogen, inhibiting the growth or activity of the plant pathogen, altering the behavior of the plant pathogen, and repelling or attracting the plant pathogen in plants, plant parts or in other agro-related settings, such as, for example, in soil.

“Microbicidal (effect)” or “microbicidal use,” as used herein, includes any effect or use of an agent, in particular, of a VHH, for controlling, modulating or interfering with the harmful activity of a plant pathogen, including killing the plant pathogen,

“Microbiostatic (effect)” or “microbiostatic use,” as used herein, includes any effect or use of an agent, in particular, of a VHH, for controlling, modulating or interfering with the harmful activity of a plant pathogen, including inhibiting the growth or activity of the plant pathogen, altering the behavior of the plant pathogen, and repelling or attracting the plant pathogen in plants, plant parts or in other agro-related settings, such as, for example, in soil.

“Antifungal (effect)” or “antifungal use,” as used herein, includes any effect or use of an agent, in particular, of a VHH, for controlling, modulating or interfering with the harmful activity of a fungus, including inhibiting the growth or activity of the fungus, altering the behavior of the fungus, and repelling or attracting the fungus in plants, plant parts or in other agro-related settings, such as in soil.

“Fungicidal (effect)” or “Fungicidal use,” as used herein, includes any effect or use of an agent, in particular, of a VHH, for controlling, modulating or interfering with the harmful activity of a fungus, including killing the fungus.

“Fungistatic (effect)” or “Fungistatic use,” as used herein, includes any effect or use of an agent, in particular, of a VHH, for controlling, modulating or interfering with the harmful activity of a fungus, including inhibiting the growth or activity of the fungus, altering the behavior of the fungus, and repelling or attracting the fungus in plants, plant parts or in other agro-related settings, such as, for example, in soil.

“Pesticidal activity” or “biocidal activity,” as used interchangeably herein, means to interfere with the harmful activity of a plant pathogen, including but not limited to killing the plant pathogen, inhibiting the growth or activity of the plant pathogen, altering the behavior of the plant pathogen, repelling or attracting the plant pathogen.

“Biostatic activity,” as used herein, means to interfere with the harmful activity of a plant pathogen, including but not limited to inhibiting the growth or activity of the plant pathogen, altering the behavior of the plant pathogen, repelling or attracting the plant pathogen.

Pesticidal, biocidal, or biostatic activity of an active ingredient, substance or principle or a composition or agent comprising a pesticidal, biocidal, or biostatic active ingredient, substance or principle, can be expressed as the minimum inhibitory activity (MIC) of an agent (expressed in units of concentration such as, e.g., mg/mL), without, however, being restricted thereto.

“Fungicidal activity,” as used herein, means to interfere with the harmful activity of a fungus, including but not limited to killing the fungus, inhibiting the growth or activity of the fungus, altering the behavior of the fungus, and repelling or attracting the fungus.

“Fungistatic activity,” as used herein, means to interfere with the harmful activity of a fungus, including but not limited to inhibiting the growth or activity of the fungus, altering the behavior of the fungus, and repelling or attracting the fungus.

Fungicidal or fungistatic activity of an active ingredient, substance or principle or a composition or agent comprising a pesticidal, biocidal, or biostatic active ingredient, substance or principle, can be expressed as the minimum inhibitory activity (MIC) of an agent (expressed in units of concentration such as, e.g., mg/mL), without, however, being restricted thereto.

As used herein, the term “antibody” refers to polyclonal antibodies, monoclonal antibodies, humanized antibodies, single-chain antibodies, and fragments thereof such as Fab, F(ab)2, Fv, and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab)2, Fv, and others that retain the antigen binding function of the antibody. Monoclonal antibodies of any mammalian species can be used in this disclosure. In practice, however, the antibodies will typically be of rat or murine origin because of the availability of rat or murine cell lines for use in making the required hybrid cell lines or hybridomas to produce monoclonal antibodies.

As used herein, the term “polyclonal antibody” refers to an antibody composition having a heterogeneous antibody population. Polyclonal antibodies are often derived from the pooled serum from immunized animals or from selected humans.

The terms “variable domain of a heavy-chain antibody” or “VHH” or “heavy chain variable domain of an antibody,” as used herein interchangeably, refer to the variable domain of the heavy chain of a heavy-chain antibody, which is naturally devoid of light chains, including but not limited to the variable domain of the heavy chain of heavy-chain antibodies of camelids or sharks.

The skilled person may understand that functional variants of VHH include any immunoglobulin single variable domain. Examples of immunoglobulin single variable domains include an immunoglobulin single variable domain selected form the group consisting of an immunoglobulin single variable domain derived from a heavy-chain antibody, an immunoglobulin single variable domain derived from a light chain variable domain sequence, an immunoglobulin single variable domain derived from a heavy chain variable domain sequence, an immunoglobulin single variable domain derived from a conventional four-chain antibody, a domain antibody, a single domain antibody, a “dAb” (Domantis/GSK), a VHH, or Nanobody (Ablynx).

As further described herein, the amino acid sequence and structure of a variable domain of a heavy-chain antibody can be considered, without however being limited thereto, to be comprised of four framework regions or “FRs,” which are referred to in the art and herein as “framework region 1” or “FR1”; as “framework region 2” or “FR2”; as “framework region 3” or “FR3”; and as “framework region 4” or “FR4,” respectively, which framework regions are interrupted by three complementarity-determining regions or “CDRs,” which are referred to in the art as “complementarity-determining region 1” or “CDR1”; as “complementarity-determining region 2” or “CDR2”; and as “complementarity-determining region 3” or “CDR3,” respectively.

As also further described herein, the total number of amino acid residues in a variable domain of a heavy-chain antibody or VHH can be in the region of 110-130, preferably is 112-115, and most preferably is 113. It should however be noted that parts, fragments or analogs of a variable domain of a heavy-chain antibody are not particularly limited as to their length and/or size, as long as such parts, fragments or analogs retain (at least part of) the functional activity, such as the pesticidal, biocidal, biostatic activity, fungicidal or fungistatic activity (as defined herein) and/or retain (at least part of) the binding specificity of the original a variable domain of a heavy-chain antibody from which these parts, fragments or analogs are derived from. Parts, fragments or analogs retaining (at least part of) the functional activity, such as the pesticidal, biocidal, biostatic activity, fungicidal or fungistatic activity (as defined herein) and/or retaining (at least part of) the binding specificity of the original variable domain of a heavy-chain antibody from which these parts, fragments or analogs are derived from are also further referred to herein as “functional fragments” of a variable domain of a heavy-chain antibody.

The amino acid residues of a variable domain of a heavy-chain antibody are numbered according to the general numbering for variable domains given by Kabat et al. (“Sequence of proteins of immunological interest,” U.S. Public Health Services, NIH Bethesda, Md., Publication No. 91), as applied to V_{HH} domains from Camelids in the article of Riechmann and Muyldermans, referred to above (see, for example, FIG. 2 of that reference). According to this numbering, FR1 of a variable domain of a heavy-chain antibody comprises the amino acid residues at positions 1-30, CDR1 of a variable domain of a heavy-chain antibody comprises the amino acid residues at positions 31-36, FR2 of a variable domain of a heavy-chain antibody comprises the amino acids at positions 36-49, CDR2 of a variable domain of a heavy-chain antibody comprises the amino acid residues at positions 50-65, FR3 of a variable domain of a heavy-chain antibody comprises the amino acid residues at positions 66-94, CDR3 of a variable domain of a heavy-chain antibody comprises the amino acid residues at positions 95-102, and FR4 of a variable domain of a heavy-chain antibody comprises the amino acid residues at positions 103-113. In this respect, it should be noted that—as is well known in the art for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDRs, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

Alternative methods for numbering the amino acid residues of a variable domain of a heavy-chain antibody are the method described by Chothia et al. (Nature 342, 877-883 (1989)), the so-called “AbM definition” and the so-called “contact definition.” However, in the present description, claims and figures, the numbering according to Kabat as applied to V_{HH} domains by Riechmann and Muyldermans will be followed, unless indicated otherwise.

For a general description of heavy-chain antibodies and the variable domains thereof, reference is made, inter alia, to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO97/49805, WO 01/21817, WO 03/035694, WO 03/054016 and WO 03/055527 of the Vlaams Instituut voor Biotechnologie (VIB); WO 03/050531 of Algonomics N.V. and Ablynx NV; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by Ablynx NV and the further published patent applications by Ablynx NV; Hamers-Casterman et al., Nature 1993 Jun. 3; 363 (6428):446-8; Davies and Riechmann, FEBS Lett. 1994 Feb. 21; 339 (3):285-90; Muyldermans et al., Protein Eng. 1994 September; 7 (9):1129-3; Davies and Riechmann, Biotechnology (NY) 1995 May; 13 (5):475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9 (6):531-7; Desmyter et al., Nat. Struct. Biol. 1996 September; 3 (9):803-11; Sheriff et al., Nat. Struct. Biol. 1996 September; 3 (9):733-6; Spinelli et al., Nat. Struct. Biol. 1996 September; 3 (9):752-7; Arbabi Ghahroudi et al., FEBS Lett. 1997 Sep. 15; 414 (3):521-6; Vu et al., Mol. Immunol. 1997 November-December; 34 (16-17):1121-31; Atarhouch et al., Journal of Carnel. Practice and Research 1997; 4:177-182; Nguyen et al., J. Mol. Biol. 1998 Jan. 23; 275 (3):413-8; Lauwereys et al., EMBO J 1998 Jul. 1; 17 (13):3512-20; Frenken et al., Res. Immunol. 1998 July-August; 149 (6):589-99; Transue et al., Proteins 1998 Sep. 1; 32 (4):515-22; Muyldermans and Lauwereys, J. Mol. Recognit. 1999 March-April; 12 (2):131-40; van der Linden et al., Biochim. Biophys. Acta 1999 Apr. 12; 1431 (1):37-46; Decanniere et al., Structure Fold. Des. 1999 Apr. 15; 7 (4):361-70; Ngyuen et al., Mol. Immunol. 1999 June; 36 (8):515-24; Woolven et al., Immunogenetics 1999 October; 50 (1-2):98-101; Riechmann and Muyldermans, J. Immunol. Methods 1999 Dec. 10; 231 (1-2):25-38; Spinelli et al., Biochemistry 2000 Feb. 15; 39 (6):1217-22; Frenken et al., J Biotechnol. 2000 Feb. 28; 78 (1): 11-21; Nguyen et al., EMBO J 2000 Mar. 1; 19 (5):921-30; van der Linden et al., J. Immunol. Methods 2000 Jun. 23; 240 (1-2):185-95; Decanniere et al., J. Mol. Biol. 2000 Jun. 30; 300 (1):83-91; van der Linden et al., J Biotechnol. 2000 Jul. 14; 80 (3):261-70; Harmsen et al., Mol. Immunol. 2000 August; 37 (10):579-90; Perez et al., Biochemistry 2001 Jan. 9; 40 (1):74-83; Conrath et al., J. Biol. Chem. 2001 Mar. 9; 276 (10):7346-50; Muyldermans et al., Trends Biochem. Sci. 2001 April; 26 (4):230-5; S. Muyldermans, J. Biotechnol. 2001 June; 74 (4):277-302; Desmyter et al., J. Biol. Chem. 2001 Jul. 13; 276 (28):26285-90; Spinelli et al., J. Mol. Biol. 2001 Aug. 3; 311 (1):123-9; Conrath et al., Antimicrob. Agents Chemother. 2001 October; 45 (10):2807-12; Decanniere et al., J. Mol. Biol. 2001 Oct. 26; 313 (3):473-8; Nguyen et al., Adv. Immunol. 2001; 79:261-96; Muruganandam et al., FASEB J. 2002 February; 16 (2):240-2; Ewert et al., Biochemistry 2002 Mar. 19; 41 (11):3628-36; Dumoulin et al., Protein Sci. 2002 March; 11 (3):500-15; Cortez-Retamozo et al., Int. J. Cancer 2002 Mar. 20; 98 (3):456-62; Su et al., Mol. Biol. Evol. 2002 March; 19 (3):205-15; J. M. van der Vaart, Methods Mol. Biol. 2002; 178:359-66; Vranken et al., Biochemistry 2002 Jul. 9; 41 (27):8570-9; Nguyen et al., Immunogenetics 2002 April; 54 (1):39-47; Renisio et al., Proteins 2002 Jun. 1; 47 (4):546-55; Desmyter et al., J. Biol. Chem. 2002 Jun. 28; 277 (26):23645-50; Ledeboer et al., J. Dairy Sci. 2002 June; 85 (6):1376-82; De Genst et al., J. Biol. Chem. 2002 Aug. 16; 277 (33):29897-907; Ferrat et al., Biochem. J. 2002 Sep. 1; 366 (Pt. 2):415-22; Thomassen et al., Enzyme and Microbiol. Technol. 2002; 30:273-8; Harmsen et al., Appl. Microbiol. Biotechnol. 2002 December; 60 (4):449-54; Jobling et al., Nat. Biotechnol. 2003 January; 21 (1):77-80; Conrath et al., Dev. Comp. Immunol. 2003 February; 27 (2):87-103; Pleschberger et al., Bioconjug. Chem. 2003 March-April; 14 (2):440-8; Lah et al., J. Biol. Chem. 2003 Apr. 18; 278 (16):14101-11; Nguyen et al., Immunology 2003 May; 109 (1):93-101; Joosten et al., Microb. Cell Fact. 2003 Jan. 30; 2 (1):1; Li et al., Proteins 2003 Jul. 1; 52 (1):47-50; Loris et al., Biol. Chem. 2003 Jul. 25; 278 (30):28252-7; van Koningsbruggen et al., J. Immunol. Methods 2003 August; 279 (1-2):149-61; Dumoulin et al., Nature 2003 Aug. 14; 424 (6950):783-8; Bond et al., J. Mol. Biol. 2003 Sep. 19; 332 (3):643-55; Yau et al., J. Immunol. Methods 2003 Oct. 1; 281 (1-2):161-75; Dekker et al., J. Virol. 2003 November; 77 (22):12132-9; Meddeb-Mouelhi et al., Toxicon. 2003 December; 42 (7):785-91; Verheesen et al., Biochim. Biophys. Acta 2003 Dec. 5; 1624 (1-3):21-8; Zhang et al., J. Mol. Biol. 2004 Jan. 2; 335 (1):49-56; Stijlemans et al., J. Biol. Chem. 2004 Jan. 9; 279 (2):1256-61; Cortez-Retamozo et al., Cancer Res. 2004 Apr. 15; 64 (8):2853-7; Spinelli et al., FEBS Lett. 2004 Apr. 23; 564 (1-2):35-40; Pleschberger et al., Bioconjug. Chem. 2004 May-June; 15 (3):664-71; Nicaise et al., Protein Sci. 2004 July; 13 (7):1882-91; Omidfar et al., Tumour Biol. 2004 July-August; 25 (4):179-87; Omidfar et al., Tumour Biol. 2004 September-December; 25 (5-6):296-305; Szynol et al., Antimicrob. Agents Chemother. 2004 September; 48 (9):3390-5; Saerens et al., J. Biol. Chem. 2004 Dec. 10; 279 (50):51965-72; De Genst et al., J. Biol. Chem. 2004 Dec. 17; 279 (51):53593-601; Dolk et al., Appl. Environ. Microbiol. 2005 January; 71 (1):442-50; Joosten et al., Appl. Microbiol. Biotechnol. 2005 January; 66 (4):384-92; Dumoulin et al., J. Mol. Biol. 2005 Feb. 25; 346 (3):773-88; Yau et al., J. Immunol. Methods 2005 February; 297 (1-2):213-24; De Genst et al., J. Biol. Chem. 2005 Apr. 8; 280 (14):14114-21; Huang et al., Eur. J. Hum. Genet. 2005 Apr. 13; Dolk et al., Proteins 2005 May 15; 59 (3):555-64; Bond et al., J. Mol. Biol. 2005 May 6; 348 (3):699-709; Zarebski et al., J. Mol. Biol. 2005 Apr. 21; [E-publication ahead of print].

Generally, it should be noted that the term “variable domain of a heavy-chain antibody,” “variable domain” or “heavy chain variable domain” as used herein in its broadest sense is not limited to a specific biological source or to a specific method of preparation. For example, as will be discussed in more detail below, the VHHs as taught herein can be obtained by expression of a nucleotide sequence encoding a naturally occurring V_{HH} domain, or by preparing a nucleic acid encoding a V_{HH} using techniques for nucleic acid synthesis, followed by expression of the nucleic acid thus obtained, or by any combination of the foregoing. Suitable methods and techniques for performing the foregoing will be clear to the skilled person based on the disclosure herein and, for example, include the methods and techniques described in more detail herein.

However, according to a specific embodiment, the variable domain of a heavy-chain antibody as disclosed herein do not have an amino acid sequence that is exactly the same as (i.e., as a degree of sequence identity of 100% with) the amino acid sequence of a naturally occurring VHH domain, such as the amino acid sequence of a naturally occurring VHH domain from a camelid or shark.

As used herein, the terms “determining,” “measuring,” “assessing,” “monitoring” and “assaying” are used interchangeably and include both quantitative and qualitative determinations.

All documents cited in the present specification are hereby incorporated by reference in their entirety. Unless otherwise defined, all terms herein used in describing the disclosure, including technical and scientific terms, have the meaning as commonly understood by one of ordinary skill in the art to which this disclosure belongs. By means of further guidance, term definitions are included to better appreciate the teaching of this disclosure.

### Transgenic Plants

This disclosure relates to a transgenic plant or plant tissue or plant cell comprising at least one polynucleotide encoding a VHH specifically binding to a sphingolipid of a fungus.

As used herein, the wording “at least one polynucleotide comprising at least one sequence encoding a VHH” and “at least one polynucleotide encoding a VHH” may be used interchangeably.

The term “transgenic plant or plant tissue” generally refers to plants or plant tissues or plant cells that have been genetically engineered to create plants with new characteristics. A transgenic plant or plant tissue or plant cell can also be identified as a genetically modified organism (GMO).

The term “transgenic plant” also encompasses commodity products derived from the transgenic plant or plant tissue or plant cell, wherein the commodity product comprises a detectable amount of the transgenic or recombinant polynucleotide and wherein the commodity product is selected from the group consisting of plant biomass, oil, meal, food, animal feed, flour, flakes, bran, lint, fiber, paper, protein, starch, silage, hulls, and processed seed, and wherein optionally the commodity product is non-regenerable.

In certain embodiments, the plant may be selected from the group consisting of maize, soya bean, alfalfa, cotton, sunflower, Brassica oil seeds such as Brassica napus (e.g., canola, rape-seed), Brassica rapa, B. juncea (e.g., (field) mustard) and Brassica carinata, Arecaceae sp. (e.g., oilpalm, coconut), rice, wheat, sugar beet, sugar cane, oats, rye, barley, millet and sorghum, triticale, flax, nuts, grapes and vine and various fruit and vegetables from various botanic taxa, e.g., Rosaceae sp. (e.g., pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g., olive tree), Actinidaceae sp., Lauraceae sp. (e.g., avocado, cinnamon, camphor), Musaceae sp. (e.g., banana trees and plantations), Rubiaceae sp. (e.g., coffee), Theaceae sp. (e.g., tea), Sterculiceae sp., Rutaceae sp. (e.g., lemons, oranges, mandarins and grapefruit); Solanaceae sp. (e.g., tomatoes, potatoes, peppers, capsicum, aubergines, tobacco), Liliaceae sp., Compositae sp. (e.g., lettuce, artichokes and chicory, including root chicory, endive or common chicory), Umbelliferae sp. (e.g., carrots, parsley, celery and celeriac), Cu-curbitaceae sp. (e.g., cucumbers, including gherkins, pumpkins, watermelons, calabashes and melons), Alliaceae sp. (e.g., leeks and onions), Cruciferae sp. (e.g., white cabbage, red cabbage, broccoli, cauliflower, Brussels sprouts, pak choi, kohlrabi, radishes, horseradish, cress and Chinese cabbage), Leguminosae sp. (e.g., peanuts, peas, lentils and beans, e.g., common beans and broad beans), Chenopodiaceae sp. (e.g., Swiss chard, fodder beet, spinach, beetroot), Linaceae sp. (e.g., hemp), Cannabeacea sp. (e.g., cannabis), Malvaceae sp. (e.g., okra, cocoa), Papaveraceae sp. (e.g., poppy), Asparagaceae sp. (e.g., asparagus); useful plants and ornamental plants in the garden and woods including turf, lawn, grass and Stevia rebaudiana, and genetically modified types of these plants.

In certain embodiments, the plant may be a crop selected from the group consisting of field crops, grasses, fruits, vegetables, lawns, trees, and ornamental plants.

In certain embodiments, the plant may be a harvestable part of the plant selected from the group consisting of a fruit, a flower, a nut, a vegetable, a fruit or vegetable with inedible peel, preferably selected from avocados, bananas, plantains, lemons, grapefruits, melons, oranges, pineapples, kiwi fruits, guavas, mandarins, mangoes and pumpkin, is preferred, more preferably bananas, oranges, lemons and peaches, in particular, bananas. In certain embodiments, the plant may be a cut flower of ornamental plants, preferably selected from Alstroemeria, Carnation, Chrysanthemum, Freesia, Gerbera, Gladiolus, baby's breath (Gypsophila spec), Helianthus, Hydrangea, Lilium, Lisianthus, roses and summer flowers. In certain embodiments, the plant may be cut grass or wood.

In certain embodiments, the plant may be a plant used for research purposes such as Arabidopsis, corn, tobacco, or poplar.

In certain embodiments, the plant may be a plant selected from the group consisting of corn, rice, wheat, barley, sorghum, millet oats, rye, triticale or other cereals, soybean, alfalfa or other leguminous crops, sugar beet, fodder beet, papaya, banana and plantains or other fruits, grapevines, nuts, oilseed rape, sunflower or other oil crops, squash cucumber, melons or other cucurbits, cotton or other fiber plants, sugarcane, palm, jatropha or other fuel crops, cabbages, tomato, pepper or other vegetables, ornamentals, shrubs, poplar, eucalyptus or other trees, evergreens, grasses, coffee plants, tea plants, tobacco plants, hop plants, rubber plants, and latex plants.

In certain preferred embodiments, the plant may be selected from the group consisting of banana, barley oat rye, canola, corn, cotton, potato, rice, soybean, tobacco, and wheat.

In certain more preferred embodiments, the plant may be selected from the group consisting of canola, corn, rice, soybean, and wheat.

In certain even more preferred embodiments, the plant may be selected from the group consisting of rice, soybean, and wheat.

Methods for the generation of transgenic plant including recombinant DNA techniques are well-known in the art.

Specifically recombinant methodologies generally involve inserting a DNA molecule expressing an amino acid sequence, protein or polypeptide of interest into an expression system to which the DNA molecule is heterologous (i.e., not normally present in the host). The heterologous DNA molecule is inserted into the expression system or vector in proper sense orientation and correct reading frame. The vector contains the necessary elements for the transcription and translation of the inserted protein-coding sequences. Transcription of DNA is dependent upon the presence of a promoter. Similarly, translation of mRNA in prokaryotes depends upon the presence of the proper prokaryotic signals, which differ from those of eukaryotes. For a review on maximizing gene expression, see Roberts and Lauer, Methods in Enzymology 68:473 (1979. Regardless of the specific regulatory sequences employed, the DNA molecule is cloned into the vector using standard cloning procedures in the art, as described by Sambrook et al, Molecular Cloning: A Laboratory Manual, Cold Springs Laboratory, Cold Springs Harbor, N.Y. (1989). Once the isolated DNA molecule encoding the protein has been cloned into an expression system, it is ready to be incorporated into a host cell. Such incorporation can be carried out by the various forms of transformation, depending upon the vector/host cell system.

In certain embodiments, this disclosure provides nucleic acid sequences capable of encoding a VHH in a transgenic plant or plant tissue or plant cell as defined herein. These nucleic acid sequences can also be in the form of a vector or a genetic construct or polynucleotide. As used herein, the terms “genetic construct” and “nucleic acid construct” are used interchangeably. The nucleic acid sequences as disclosed herein may be synthetic or semi-synthetic sequences, nucleotide sequences that have been isolated from a library (and, in particular, an expression library), nucleotide sequences that have been prepared by PCR using overlapping primers, or nucleotide sequences that have been prepared using techniques for DNA synthesis known per se. In certain embodiments, the polynucleotide may comprise nucleic acid sequence of SEQ ID NO:336 and/or SEQ ID NO:337.

The genetic constructs as disclosed herein may be DNA or RNA, and are preferably double-stranded DNA. The genetic constructs of the disclosure may also be in a form suitable for transformation of the intended host cell or host organism in a form suitable for integration into the genomic DNA of the intended host cell or in a form suitable for independent replication, maintenance and/or inheritance in the intended host organism. For instance, the genetic constructs of the disclosure may be in the form of a vector, such as, for example, a plasmid, cosmid, YAC, a viral vector or transposon. In particular, the vector may be an expression vector, i.e., a vector that can provide for expression in vitro and/or in vivo (e.g., in a suitable host cell, host organism and/or expression system).

Accordingly, in another further aspect, this disclosure also provides vectors comprising one or more nucleic acid sequences of the disclosure.

Also disclosed are chimeric genes comprising the following operably linked DNA elements: a) a plant expressible promoter, b) a DNA region, which, when transcribed, yields a mRNA molecule capable of being translated into a polypeptide and c) a 3′ end region comprising transcription termination and polyadenylation signals functioning in cells of the plant.

A “chimeric gene” or “chimeric construct” is a recombinant nucleic acid sequence in which a promoter (e.g., a plant expressible promoter) or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence when introduced into a cell such as a plant cell. The regulatory nucleic acid sequence of the chimeric gene is not normally operatively linked to the associated nucleic acid sequence as found in nature.

In certain embodiments, the polynucleotide may comprise a promoter suitable for expression in plants, a plant tissue or plant cell specific promoter, or an inducible promoter.

The terms “plant promoter” or “promoter suitable for expression in plants” as used herein refers to a nucleic acid sequence comprising regulatory elements, which mediate the expression of a coding sequence in plant cells. For expression in plants, the nucleic acid molecule must be linked operably to or comprise a suitable promoter that expresses the gene at the right point in time and with the required spatial expression pattern.

The term “operably linked” as used herein refers to a functional linkage between the promoter sequence and the gene of interest, such that the promoter sequence is able to initiate transcription of the gene of interest.

Plant expressible promoters comprise nucleic acid sequences that are able to direct the expression of a transgene in a plant. Examples of plant expressible promoters are constitutive promoters that are transcriptionally active during most, but not necessarily all, phases of growth and development and under most environmental conditions, in at least one cell, tissue or organ, other promoters are inducible promoters, other examples are tissue specific promoters, still other examples are abiotic stress inducible promoters.

The term “plant tissue or plant cell specific promoter” refers to promoters that are transcriptionally active in a specific type of plant cells or plant tissues.

The term “inducible promoter” refers to promoters that allow regulating gene expression levels at particular stages of plant development and in particular tissues of interest. Examples of inducible systems include AlcR/AlcA (ethanol inducible); GR fusions, GVG, and pOp/LhGR (dexamethasone inducible); XVE/OlexA (beta-estradiol inducible); and heat shock induction.

The chimeric gene (or the expression cassette) when transformed in a plant expresses a nucleic acid, which results in expression of a protein.

Also disclosed is a recombinant vector that comprises an expression cassette (or a chimeric gene) as herein described before.

The term “terminator” encompasses a control sequence that is a DNA sequence at the end of a transcriptional unit that signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

In certain embodiments of the transgenic plants or plant tissues or plant cells, methods, or uses, as taught herein, the polynucleotide may comprise at least one sequence encoding an antibody fragment consisting of a variable domain of a heavy-chain antibody (VHH). In certain embodiments, the polynucleotide may encode an antibody fragment consisting of a variable domain of a heavy-chain antibody (VHH).

In certain embodiments, the polynucleotide may not comprise a sequence encoding a light chain of an antibody, such as an immunoglobulin light chain. In certain embodiments, the polynucleotide may not encode an immunoglobulin.

In certain embodiments, the transgenic plant or plant tissue or plant cell may not comprise any polynucleotide (comprising at least one sequence) encoding a light chain of an antibody, such as an immunoglobulin light chain. In certain embodiments, the transgenic plant or plant tissue or plant cell may not comprise any polynucleotide (comprising at least one sequence) encoding an immunoglobulin.

In certain embodiments, the polynucleotide may comprise at least one sequence encoding a targeting signal for secretion, for location to the cytoplasm, or for location to cellular compartments or organelles, such as the ER lumen, the apoplast, the vacuole, or intra- and/or exterior membranes.

Examples of a targeting signal for secretion include the 2S2 signal peptide. Examples of a targeting signal for location to cellular compartments or organelles, such as the ER lumen include the ER retention signal (KDEL).

In certain embodiments, the polynucleotide may comprise an ATG start codon for location to the cytoplasm.

In certain embodiments, the polynucleotide may encode the VHH as such, as a combination with one or more identical or different VHHs, or as a combination with one or more identical or different VHHs with a fragment crystallizable region (Fc region); optionally with a spacer.

The term “fragment crystallizable region” or “Fc region” refers to the tail region of an antibody. The Fc region may interact with cell surface receptors called Fc receptors and some proteins of the complement system. The Fc region of IgG, IgA and IgD antibody isotypes is composed of two identical protein fragments, derived from the second and third constant domains of the antibody's two heavy chains. The Fc region of IgM and IgE contains three heavy chain constant domains (CH domains 2 to 4) in each polypeptide chain.

Such an Fc region may advantageously enhance the stability of the VHH and/or may increase the expression of the VHH.

In certain embodiments, the Fc region may be an Fc region of an IgG antibody, an IgA antibody, an IgD antibody, an IgM antibody, or an IgE antibody. In certain preferred embodiments, the Fc region may be an Fc region of an IgG antibody, an IgA antibody, or an IgD antibody. In certain more preferred embodiments, the Fc region may be an Fc region of an IgG antibody.

In certain embodiments, the Fc region may be an Fc region of a mouse antibody (i.e., mouse Fc region). In certain preferred embodiments, the Fc region may be an Fc region of a human antibody (i.e., human Fc region).

In certain preferred embodiments, the Fc region may be the Fc region of mouse IgG3 antibody. In certain preferred embodiments, the Fc region may be the Fc region of the human IgG1 antibody (hGFc; Van Droogenbroeck et al., 2007, Proc. Natl. Acad. Sci. U.S.A. 104 (4):1430-5).

The term “monovalent” as used herein refers to an antibody or antibody fragment comprising one binding site.

The term “bivalent” as used herein refers to an antibody or antibody fragment comprising two or more binding sites.

The terms “spacer” or “linker,” as used herein interchangeably, refer to at least one amino acid spatially separating at least two VHHs. An exemplary spacer includes the 9GS spacer consisting of GGGGSGGGS (SEQ ID NO:352).

In certain embodiments of the transgenic plants or plant tissues or plant cells, methods, or uses, as taught herein, the VHHs as disclosed herein may be optionally linked to one or more further groups, moieties, or residues via one or more linkers. These one or more further groups, moieties or residues can serve for binding to other targets of interest. It should be clear that such further groups, residues, moieties and/or binding sites may or may not provide further functionality to the VHH as disclosed herein and may or may not modify the properties of the VHH as disclosed herein. Such groups, residues, moieties or binding units may also, for example, be chemical groups that can be biologically active.

These groups, moieties or residues are, in particular embodiments, linked N- or C-terminally to the VHH as disclosed herein.

For example, the introduction or linkage of such functional groups to a variable domain heavy-chain antibody can result in an increase in the solubility and/or the stability of the variable domain heavy-chain antibody, in a reduction of the toxicity of the variable domain heavy-chain antibody, or in the elimination or attenuation of any undesirable side effects of the variable domain heavy-chain antibody, and/or in other advantageous properties.

In particular embodiments, the one or more groups, residues, moieties are linked to the variable domain heavy-chain antibody via one or more suitable linkers or spacers.

In further particular embodiments, two or more target-specific variable domain heavy-chain antibodies disclosed herein may be linked to each other or may be interconnected. In particular embodiments, the two or more variable domain heavy-chain antibodies may be linked to each other via one or more suitable linkers or spacers. Suitable spacers or linkers for use in the coupling of different variable domain heavy-chain antibodies as disclosed herein will be clear to the skilled person and may generally be any linker or spacer used in the art to link peptides and/or proteins.

Some particularly suitable linkers or spacers include, for example, but are not limited to, polypeptide linkers such as glycine linkers, serine linkers, mixed glycine/serine linkers, glycine- and serine-rich linkers or linkers composed of largely polar polypeptide fragments, or homo- or heterobifunctional chemical crosslinking compounds such as glutaraldehyde or, optionally PEG-spaced, maleimides or NHS esters.

For example, a polypeptide linker or spacer may be a suitable amino acid sequence having a length between 1 and 50 amino acids, such as between 1 and 30, and, in particular, between 1 and 10 amino acid residues. It should be clear that the length, the degree of flexibility and/or other properties of the linker(s) may have some influence on the properties of the VHHs, including but not limited to the affinity, specificity or avidity for the fungal target. It should be clear that when two or more linkers are used, these linkers may be the same or different. In the context and disclosure of this application, the person skilled in the art will be able to determine the optimal linkers for the purpose of coupling VHHs as disclosed herein without any undue experimental burden.

“Selectable marker,” “selectable marker gene” or “reporter gene” includes any gene that confers a phenotype on a cell in which it is expressed to facilitate the identification and/or selection of cells that are transfected or transformed with a nucleic acid construct of the disclosure. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptl1 that phosphorylates neomycin and kanamycin, or hpt, phosphorylating hygromycin, or genes conferring resistance to, for example, bleomycin, streptomycin, tetracycline, chloramphenicol, ampicillin, gentamycin, geneticin (G418), spectinomycin or blasticidin), to herbicides (for example, bar that provides resistance to BASTA®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or antinutritive markers such as the resistance to 2-deoxyglucose). Expression of visual marker genes results in the formation of color (for example, β-glucuronidase, GUS or β-galactosidase with its colored substrates, for example, X-Gal), luminescence (such as the luciferin/luciferase system) or fluorescence (Green Fluorescent Protein, GFP, and derivatives thereof). This list represents only a small number of possible markers. The skilled worker is familiar with such markers.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can, for example, be used in mutants, in which these genes are not functional by, for example, deletion by conventional methods. Furthermore, nucleic acid molecules encoding a selectable marker can be introduced into a host cell on the same vector that comprises the sequence encoding the polypeptides of the disclosure or used in the methods of the disclosure, or else in a separate vector. Cells that have been stably transfected with the introduced nucleic acid can be identified, for example, by selection (for example, cells that have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the disclosure for introducing the nucleic acids advantageously employs techniques that enable the removal or excision of these marker genes. One such a method is what is known as co-transformation. The co-transformation method employs two vectors simultaneously for the transformation, one vector bearing the nucleic acid according to the disclosure and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e., the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed that make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem. 275, 2000:22255-22267; Velmurugan et al., J. Cell. Biol. 149, 2000:553-566). A site-specific integration into the plant genome of the nucleic acid sequences according to the disclosure is possible.

A transgenic plant for the purposes of the disclosure is thus understood as meaning, as above, that the nucleic acids used in the method of the disclosure are not present in, or originating from, the genome of the plant, or are present in the genome of the plant but not at their natural locus in the genome of the plant, it being possible for the nucleic acids to be expressed homologously or heterologously. However, as mentioned, transgenic also means that, while the nucleic acids according to the disclosure or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the disclosure at an unnatural locus in the genome, i.e., homologous or, heterologous expression of the nucleic acids takes place. Preferred transgenic plants are mentioned herein.

The term “expression” or “gene expression” means the transcription of a specific gene or specific genes or specific genetic construct. The term “expression” or “gene expression,” as used herein, refers to transcription of a polynucleotide or gene or genetic construct into structural RNA (rRNA, tRNA) or mRNA with or without subsequent translation of the latter into a protein. The process includes transcription of DNA and processing of the resulting mRNA product.

The term “increased expression” or “overexpression” as used herein means any form of expression that is additional to the original wild-type expression level. For the purposes of this disclosure, the original wild-type expression level might also be zero, i.e., absence of expression or immeasurable expression.

Methods for increasing expression of genes or gene products are well documented in the art and include, for example, overexpression driven by appropriate promoters (as described herein before), the use of transcription enhancers or translation enhancers. Isolated nucleic acids that serve as promoter or enhancer elements may be introduced in an appropriate position (typically upstream) of a non-heterologous form of a polynucleotide so as to upregulate expression of a nucleic acid encoding the polypeptide of interest. If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell Biol. 8:4395-4405; Callis et al. (1987) Genes Dev. 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

The term “encoding,” as used herein, refers to the transcription of a polynucleotide or gene or genetic construct into structural RNA (rRNA, tRNA) or mRNA with the subsequent translation of the latter into a protein.

The term “introduction” or “transformation” as referred to herein encompass the transfer of an exogenous polynucleotide or chimeric gene (or expression cassette) into a host cell, irrespective of the method used for transfer. Plant tissue or plant cells capable of subsequent clonal propagation, whether by organogenesis or embryogenesis, may be transformed with a genetic construct of this disclosure and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen, and microprojection. Methods may be selected from the calcium/polyethylene glycol method for protoplasts (F. A. Krens et al., (1982) Nature 296:72-74; I. Negrutiu et al. (1987) Plant Mol. Biol. 8:363-373); electroporation of protoplasts (R. D. Shillito et al. (1985) Bio/Technol. 3:1099-1102); microinjection into plant material (A. Crossway et al. (1986) Mol. Gen. Genet. 202:179-185); DNA or RNA-coated particle bombardment (T. M. Klein et al. (1987) Nature 327:70) infection with (non-integrative) viruses and the like.

Transgenic plants, including transgenic crop plants, are preferably produced via Agrobacterium-mediated transformation. An advantageous transformation method is the transformation inplanta. To this end, it is possible, for example, to allow the agrobacteria to act on plant seeds or to inoculate the plant meristem with agrobacteria. It has proved particularly expedient in accordance with the disclosure to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well-known methods for rice transformation, such as those described in any of the following: European patent application EP1198985, Aldemita and Hodges (Planta 199:612-617, 1996); Chan et al. (Plant Mol. Biol. 22 (3):491-506, 1993), Hiei et al. (Plant J. 6 (2):271-282, 1994), which disclosures are incorporated by reference herein as if fully set forth. In the case of corn transformation, the preferred method is as described in either Ishida et al. (Nat. Biotechnol. 14 (6):745-50, 1996) or Frame et al. (Plant Physiol. 129 (1):13-22, 2002), which disclosures are incorporated by reference herein as if fully set forth. The methods are further described by way of example in B. Jenes et al., “Techniques for Gene Transfer” in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42:205-225 (1991)). The nucleic acids or the construct to be expressed is preferably cloned into a vector, which is suitable for transforming Agrobacterium tumefaciens, for example, pBin19 (Bevan et al. (1984) Nucl. Acids Res. 12-8711) or pMP90 (Koncz and Schell (1986) Mol. Gen. Genet. 204:383-396). Agrobacteria transformed by such a vector can then be used in known manner for the transformation of plants, such as plants used as a model, like Arabidopsis (Arabidopsis thaliana is within the scope of this disclosure, but not considered as a crop plant), or crop plants such as, by way of example, tobacco plants, for example, by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16:9877, or is known inter alia from F. F. White, “Vectors for Gene Transfer in Higher Plants,” in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and, in particular, those cells that develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (K. A. Feldman and M. D. Marks (1987) Mol. Gen. Genet. 208:1-9; K. Feldmann (1992) in C. Koncz, N.-H. Chua and J. Shell, eds, Methods in Arabidopsis Research, Word Scientific, Singapore, pp. 274-289). Alternative methods are based on the repeated removal of the inflorescences and incubation of the excision site in the center of the rosette with transformed agrobacteria, whereby transformed seeds can likewise be obtained at a later point in time (Chang (1994) Plant J. 5:551-558; Katavic (1994) Mol. Gen. Genet. 245:363-370). However, an especially effective method is the vacuum infiltration method with its modifications such as the “floral dip” method. In the case of vacuum infiltration of Arabidopsis, intact plants under reduced pressure are treated with an agrobacterial suspension (N. Bechthold (1993) C.R. Acad. Sci. Paris Life Sci. 316:1194-1199), while in the case of the “floral dip” method the developing floral tissue is incubated briefly with a surfactant-treated agrobacterial suspension (S. J. Clough and A. F. Bent (1998) The Plant J. 16:735-743). A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process that has been schematically displayed in Klaus et al., 2004 (Nature Biotechnology 22 (2):225-229). Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001), “Transgenic plastids in basic research and plant biotechnology,” J. Mol. Biol. 2001 Sep. 21; 312 (3):425-38, or P. Maliga (2003), “Progress towards commercialization of plastid transformation technology,” Trends Biotechnol. 21:20-28. Further, biotechnological progress has recently been reported in the form of marker-free plastid transformants, which can be produced by a transient co-integrated maker gene (Klaus et al., 2004, Nature Biotechnology 22 (2):225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers that are encoded by plant-expressible genes co-transferred with the gene of interest, following which, the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In certain embodiments, the transgenic plant or plant tissue or plant cell may have an increased or enhanced level of a VHH as taught herein relative to (i.e., compared with) a non-modified (i.e., non-transformed or untransformed, such as wild-type) plant or plant tissue.

The level of a VHH as taught herein can be determined by any method known in the art for measuring the concentration of a protein. For instance, the level of a VHH as taught herein can be determined by an enzyme-linked immunosorbent assay (ELISA). The level of a VHH can be expressed as a percentage of the amount of VHH relative to the total protein amount.

The terms “quantity,” “amount” and “level” are synonymous and generally well-understood in the art. With respect to proteins, the terms may particularly refer to an absolute quantification of the protein in a sample, or to a relative quantification of the protein in the sample, i.e., relative to another value such as relative to a reference value (e.g., the total protein content).

In certain embodiments, the transgenic plant or plant tissue or plant cell may have a level of a VHH as taught herein, which is at least 0.001% of the amount of total soluble protein in the transgenic plant or plant tissue or plant cell, in particular, in an extract of the transgenic plant or plant tissue. For example, the transgenic plant or plant tissue or plant cell may have a level of a VHH as taught herein, which is at least 0.005%, at least 0.01%, at least 0.05%, at least 0.1%, at least 0.2%, at least 0.3%, at least 0.4%, or at least 0.5% of the amount of total soluble protein in the transgenic plant or plant tissue or plant cell, in particular, in an extract of the transgenic plant or plant tissue.

Total soluble proteins of plants or plant tissues or plant cells can be extracted. Routine procedures can be used to determine the amount of total soluble protein in extracts of plants or plant tissues. Preferably, the protein concentration is determined by a colorimetric method, such as Bradford analysis known in the art. A Western blot using anti-VHH antibody fragment antibodies can be used to verify that VHH antibody fragments are expressed in transgenic plants. Preferably, anti-histidine antibodies are used to detect VHH that carry a hexahistidine tag. Preferably, anti-Fc antibodies are used to detect VHH fused to antibody Fc fragments. The concentration of VHH in a sample can be calculated comparing samples with a standard series of VHH in known amounts. The level of VHH expression can be calculated from the VHH and total soluble protein concentration.

In one aspect, the applicants herein have identified transgenic plants or plant tissues or plant cells comprising at least one VHH, which can specifically bind to a sphingolipid of a fungus and bind to a sphingolipid of a plant pathogenic fungus. Importantly, through this interaction with a specific molecular structure of the plant pathogenic fungus, the transgenic plant or plant tissue or plant cell disclosed herein is capable of controlling, modulating, inhibiting, preventing or reducing one or more biological activities of the plant pathogen, such that the growth of the plant pathogen is controlled, modulated, inhibited, prevented or reduced. In certain embodiments, the transgenic plants or plant tissues or plant cells as taught herein are capable of killing a plant pathogenic fungus through the specific interaction of at least one VHH, which can specifically bind to a sphingolipid of a fungus and which is expressed in the plant or plant tissue.

Accordingly, the transgenic plants or plant tissues or plant cells as disclosed herein can be used to modulate, such as to change, decrease or inhibit, the biological function of a plant pathogenic fungus by binding to a binding site present on a sphingolipid target of that plant pathogenic fungus thereby affecting the natural biological activities (such as, but not limited to, growth) of the plant pathogenic fungus and/or one or more biological pathways in which the structural target of that plant pathogenic fungus is involved.

In certain embodiments of the transgenic plants or plant tissues or plant cells, methods, or uses, as taught herein, the VHHs as taught herein may be capable of specific binding (as defined herein) to a plant pathogen target or a plant pathogen antigen; and more preferably capable of binding to a plant pathogen target or a plant pest antigen or plant pathogen antigen with an affinity as defined herein (suitably measured and/or expressed as a K_{D}-value (actual or apparent), a K_{A}-value (actual or apparent), a kₒₙ-rate and/or a k_{off}-rate, or alternatively as an IC₅₀ value, as further described herein).

In particular embodiments, the disclosure provides a transgenic plant or plant tissue or plant cell, for combating plant pests, more particularly a plant fungus, which comprises at least one polypeptide or amino acid sequence of between 80 and 200 amino acids as the active substance.

In certain embodiments, the disclosure provides a transgenic plant or plant tissue or plant cell, for combating plant pests, which comprises at least two polypeptides or at least two amino acid sequences of between 80 and 200 amino acids as the active substance.

In certain embodiments, the disclosure provides a transgenic plant or plant tissue or plant cell, for combating plant pests, which comprises at least three polypeptides or at least three amino acid sequences of between 80 and 200 amino acids as the active substance.

The transgenic plant or plant tissue or plant cell according to the disclosure for combating plant pests, as defined before, means that the transgenic plant or plant tissue or plant cell, more in particular, the VHH as defined before, encoded in the transgenic plant or plant tissue or plant cell, is able to interfere with, preferably to reduce or to arrest, the harmful effects of one or more plant pests on one or more plants, preferably crops.

Thus, in one embodiment, the transgenic plant or plant tissue or plant cell comprises a polypeptide of between 80 and 200 amino acids as the active substance.

In more specific embodiments the transgenic plant or plant tissue or plant cell comprises a polypeptide of between 80-100 amino acids, 800-120 amino acids, 80-140 amino acids, 80-160 amino acids or 80-180 amino acids.

In yet another embodiment the transgenic plant or plant tissue or plant cell comprises a polypeptide of between 100-200 amino acids, 100-180 amino acids, 100-160 amino acids, 100-150 amino acids, 100-140 amino acids or 100-120 amino acids.

In yet another embodiment the transgenic plant or plant tissue or plant cell comprises a polypeptide of between 110-200 amino acids, 110-180 amino acids, 110-160 amino acids, 110-140 amino acids or 110-130 amino acids.

In yet another embodiment, the transgenic plant or plant tissue or plant cell comprises a polypeptide of between 120-200 amino acids, 120-180 amino acids, 120-160 amino acids, or 120-140 amino acids.

In yet another embodiment, the transgenic plant or plant tissue or plant cell comprises a polypeptide of between 140-200 amino acids, 140-180 amino acids, or 140-160 amino acids.

In yet another embodiment, the transgenic plant or plant tissue or plant cell comprises a polypeptide of between 160-200 amino acids or 160-180 amino acids.

The at least one variable domain of a heavy-chain antibody (VHH) comprised in the transgenic plant or plant tissue or plant cell disclosed herein can be a naturally occurring polypeptide, or alternatively can be entirely artificially designed. Non-limiting examples of such naturally occurring polypeptides include heavy chain antibodies (hcAb).

According to particular embodiments, the disclosure provides a number of stretches of amino acid residues (i.e., small peptides) that are particularly suited for binding to a sphingolipid antigen or a sphingolipid target, such as but not limited to a fungal sphingolipid antigen or a fungal sphingolipid target.

These stretches of amino acid residues may be present in, and/or may be incorporated into, the VHH as disclosed herein, in particular, in such a way that they form (part of) the antigen binding site of that VHH. As these stretches of amino acid residues were first generated as CDR sequences of antibodies, such as heavy chain antibodies, or of V_{H} or V_{HH} sequences that were raised against a sphingolipid target (or may be based on and/or derived from such CDR sequences, as further described herein), they will also generally be referred to herein as “CDR sequences” (i.e., as CDR1 sequences, CDR2 sequences and CDR3 sequences, respectively). It should however be noted that the disclosure in its broadest sense is not limited to a specific structural role or function that these stretches of amino acid residues may have in the VHH as disclosed herein, as long as these stretches of amino acid residues allow the VHHs as disclosed herein to specifically bind to a sphingolipid target. Thus, generally, the disclosure in its broadest sense relates to transgenic plant or plant tissues or plant cells comprising at least one polynucleotide encoding a variable domain of a heavy-chain antibody (VHH) that is capable of binding to a sphingolipid target and that comprises a combination of CDR sequences as described herein.

Thus, in certain embodiments, the VHHs as disclosed herein may be variable domains that comprise at least one amino acid sequence that is chosen from the group consisting of the CDR1 sequences, CDR2 sequences and CDR3 sequences that are described herein. In particular, a heavy chain variable domain as disclosed herein may comprise at least one antigen binding site, wherein the antigen binding site comprises at least one combination of a CDR1 sequence, a CDR2 sequence and a CDR3 sequence that are described herein.

Any variable domain of a heavy-chain antibody as taught herein and having one these CDR sequence combinations is preferably such that it can specifically bind (as defined herein) to a sphingolipid target or a sphingolipid antigen, and more in particular, such that it specifically binds to a sphingolipid of a plant pathogen, in particular, with dissociation constant (Kd) of 10⁻⁸ moles/liter or less of the variable domain in solution.

Specific binding of a variable domain of a heavy-chain antibody to a sphingolipid target can be determined in any suitable manner known per se, including, for example, biopanning, Scatchard analysis and/or competitive binding assays, such as radioimmunoassays (RIA), enzyme immunoassays (EIA) and sandwich competition assays, and the different variants thereof known in the art.

In a preferred embodiment, the polypeptide of between 80 and 200 amino acids, is obtained by affinity selection against a particular pest target molecule and the polypeptide has a high affinity for the pest target molecule: typically, the dissociation constant of the binding between the polypeptide and its pest target molecule is lower than 10⁻⁵ M, more preferably, the dissociation constant is lower than 10⁻⁶ M, even more preferably, the dissociation constant is lower than 10⁻⁷ M, most preferably, the dissociation constant is lower than 10⁻⁸ M.

In particular embodiments, the at least one variable domain of a heavy-chain antibody as taught herein has a minimum inhibitory concentration (MIC) value for the plant pathogenic fungus of 1.0 μg/mL or less of the variable domain in solution.

Also disclosed herein are polypeptides of between 80 and 200 amino acids or a sub-range as disclosed herein before, obtained by affinity selection to a specific plant pest target, which is able to inhibit the growth and/or the activity of a crop pest at a minimum inhibitory concentration of about 0.00001 to 1 μM. In specific embodiments the minimum inhibitory concentrations are between 0.0001 to 1 μM, are between 0.001 to 1 μM, between 0.01 to 1 μM, between 0.1 to 1 μM, between 0.0001 to 0.1 μM, between 0.001 to 0.1 μM, between 0.01 to 0.1 M, between 0.00001 to 0.01 μM, between 0.0001 to 0.01 μM, between 0.001 to 0.01 μM.

The Minimal Inhibitory Concentration or the MIC value is the lowest concentration of an agent such as a polypeptide that inhibits the visible growth of the crop or plant pest after incubation. For example, the minimum fungicidal concentration (MFC) is considered as the lowest concentration of polypeptide that prevents growth and reduces the fungal inoculum by a 99.90% within 24 hours. MFCs (Minimal Fungal Concentrations) can be determined on agar plates but can also be conveniently determined in fluids (e.g., in microwell plates) depending on the type of the fungus and the assay conditions.

In certain embodiments of the transgenic plants or plant tissues or plant cells, methods, or uses, as taught herein, the polynucleotide may comprise a sequence encoding a VHH comprising, consisting of, or consisting essentially of a CDR1, CDR2 and CDR3 region chosen from the list of comprising:
a CDR1 region having SEQ ID NO:85, a CDR2 region having has SEQ ID NO:169, and a CDR3 region having SEQ ID NO:253, and/or
a CDR1 region having SEQ ID NO:86, a CDR2 region having has SEQ ID NO:170, and a CDR3 region having SEQ ID NO:254, and/or
a CDR1 region having SEQ ID NO:87, a CDR2 region having has SEQ ID NO:171, and a CDR3 region having SEQ ID NO:255, and/or
a CDR1 region having SEQ ID NO:88, a CDR2 region having has SEQ ID NO:172, and a CDR3 region having SEQ ID NO:256, and/or
a CDR1 region having SEQ ID NO:89, a CDR2 region having has SEQ ID NO:173, and a CDR3 region having SEQ ID NO:257, and/or
a CDR1 region having SEQ ID NO:90, a CDR2 region having has SEQ ID NO:174, and a CDR3 region having SEQ ID NO:258, and/or
a CDR1 region having SEQ ID NO:91, a CDR2 region having has SEQ ID NO:175, and a CDR3 region having SEQ ID NO:259, and/or
a CDR1 region having SEQ ID NO:92, a CDR2 region having has SEQ ID NO:176, and a CDR3 region having SEQ ID NO:260, and/or
a CDR1 region having SEQ ID NO:93, a CDR2 region having has SEQ ID NO:177, and a CDR3 region having SEQ ID NO:261, and/or
a CDR1 region having SEQ ID NO:94, a CDR2 region having has SEQ ID NO:178, and a CDR3 region having SEQ ID NO:262, and/or
a CDR1 region having SEQ ID NO:95, a CDR2 region having has SEQ ID NO:179, and a CDR3 region having SEQ ID NO:263, and/or
a CDR1 region having SEQ ID NO:96, a CDR2 region having has SEQ ID NO:180, and a CDR3 region having SEQ ID NO:264, and/or
a CDR1 region having SEQ ID NO:97, a CDR2 region having has SEQ ID NO:181, and a CDR3 region having SEQ ID NO:265, and/or
a CDR1 region having SEQ ID NO:98, a CDR2 region having has SEQ ID NO:182, and a CDR3 region having SEQ ID NO:266, and/or
a CDR1 region having SEQ ID NO:99, a CDR2 region having has SEQ ID NO:183, and a CDR3 region having SEQ ID NO:267, and/or
a CDR1 region having SEQ ID NO:100, a CDR2 region having has SEQ ID NO:184, and a CDR3 region having SEQ ID NO:268, and/or
a CDR1 region having SEQ ID NO:101, a CDR2 region having has SEQ ID NO:185, and a CDR3 region having SEQ ID NO:269, and/or
a CDR1 region having SEQ ID NO:102, a CDR2 region having has SEQ ID NO:186, and a CDR3 region having SEQ ID NO:270, and/or
a CDR1 region having SEQ ID NO:103, a CDR2 region having has SEQ ID NO:187, and a CDR3 region having SEQ ID NO:271, and/or
a CDR1 region having SEQ ID NO:104, a CDR2 region having has SEQ ID NO:188, and a CDR3 region having SEQ ID NO:272, and/or
a CDR1 region having SEQ ID NO:105, a CDR2 region having has SEQ ID NO:189, and a CDR3 region having SEQ ID NO:273, and/or
a CDR1 region having SEQ ID NO:106, a CDR2 region having has SEQ ID NO:190, and a CDR3 region having SEQ ID NO:274, and/or
a CDR1 region having SEQ ID NO:107, a CDR2 region having has SEQ ID NO:191, and a CDR3 region having SEQ ID NO:275, and/or
a CDR1 region having SEQ ID NO:108, a CDR2 region having has SEQ ID NO:192, and a CDR3 region having SEQ ID NO:276, and/or
a CDR1 region having SEQ ID NO:109, a CDR2 region having has SEQ ID NO:193, and a CDR3 region having SEQ ID NO:277, and/or
a CDR1 region having SEQ ID NO:110, a CDR2 region having has SEQ ID NO:194, and a CDR3 region having SEQ ID NO:278, and/or
a CDR1 region having SEQ ID NO:111, a CDR2 region having has SEQ ID NO:195, and a CDR3 region having SEQ ID NO:279, and/or
a CDR1 region having SEQ ID NO:112, a CDR2 region having has SEQ ID NO:196, and a CDR3 region having SEQ ID NO:280, and/or
a CDR1 region having SEQ ID NO:113, a CDR2 region having has SEQ ID NO:197, and a CDR3 region having SEQ ID NO:281, and/or
a CDR1 region having SEQ ID NO:114, a CDR2 region having has SEQ ID NO:198, and a CDR3 region having SEQ ID NO:282, and/or
a CDR1 region having SEQ ID NO:115, a CDR2 region having has SEQ ID NO:199, and a CDR3 region having SEQ ID NO:283, and/or
a CDR1 region having SEQ ID NO:116, a CDR2 region having has SEQ ID NO:200, and a CDR3 region having SEQ ID NO:284, and/or
a CDR1 region having SEQ ID NO:117, a CDR2 region having has SEQ ID NO:201, and a CDR3 region having SEQ ID NO:285, and/or
a CDR1 region having SEQ ID NO:118, a CDR2 region having has SEQ ID NO:202, and a CDR3 region having SEQ ID NO:286, and/or
a CDR1 region having SEQ ID NO:119, a CDR2 region having has SEQ ID NO:203, and a CDR3 region having SEQ ID NO:287, and/or
a CDR1 region having SEQ ID NO:120, a CDR2 region having has SEQ ID NO:204, and a CDR3 region having SEQ ID NO:288, and/or
a CDR1 region having SEQ ID NO:121, a CDR2 region having has SEQ ID NO:205, and a CDR3 region having SEQ ID NO:289, and/or
a CDR1 region having SEQ ID NO:122, a CDR2 region having has SEQ ID NO:206, and a CDR3 region having SEQ ID NO:290, and/or
a CDR1 region having SEQ ID NO:123, a CDR2 region having has SEQ ID NO:207, and a CDR3 region having SEQ ID NO:291, and/or
a CDR1 region having SEQ ID NO:124, a CDR2 region having has SEQ ID NO:208, and a CDR3 region having SEQ ID NO:292, and/or
a CDR1 region having SEQ ID NO:125, a CDR2 region having has SEQ ID NO:209, and a CDR3 region having SEQ ID NO:293, and/or
a CDR1 region having SEQ ID NO:126, a CDR2 region having has SEQ ID NO:210, and a CDR3 region having SEQ ID NO:294, and/or
a CDR1 region having SEQ ID NO:127, a CDR2 region having has SEQ ID NO:211, and a CDR3 region having SEQ ID NO:295, and/or
a CDR1 region having SEQ ID NO:128, a CDR2 region having has SEQ ID NO:212, and a CDR3 region having SEQ ID NO:296, and/or
a CDR1 region having SEQ ID NO:129, a CDR2 region having has SEQ ID NO:213, and a CDR3 region having SEQ ID NO:297, and/or
a CDR1 region having SEQ ID NO:130, a CDR2 region having has SEQ ID NO:214, and a CDR3 region having SEQ ID NO:298, and/or
a CDR1 region having SEQ ID NO:131, a CDR2 region having has SEQ ID NO:215, and a CDR3 region having SEQ ID NO:299, and/or
a CDR1 region having SEQ ID NO:132, a CDR2 region having has SEQ ID NO:216, and a CDR3 region having SEQ ID NO:300, and/or
a CDR1 region having SEQ ID NO:133, a CDR2 region having has SEQ ID NO:217, and a CDR3 region having SEQ ID NO:301, and/or
a CDR1 region having SEQ ID NO:134, a CDR2 region having has SEQ ID NO:218, and a CDR3 region having SEQ ID NO:302, and/or
a CDR1 region having SEQ ID NO:135, a CDR2 region having has SEQ ID NO:219, and a CDR3 region having SEQ ID NO:303, and/or
a CDR1 region having SEQ ID NO:136, a CDR2 region having has SEQ ID NO:220, and a CDR3 region having SEQ ID NO:304, and/or
a CDR1 region having SEQ ID NO:137, a CDR2 region having has SEQ ID NO:221, and a CDR3 region having SEQ ID NO:305, and/or
a CDR1 region having SEQ ID NO:138, a CDR2 region having has SEQ ID NO:222, and a CDR3 region having SEQ ID NO:306, and/or
a CDR1 region having SEQ ID NO:139, a CDR2 region having has SEQ ID NO:223, and a CDR3 region having the amino acid sequence NRY, and/or
a CDR1 region having SEQ ID NO:140, a CDR2 region having has SEQ ID NO:224, and a CDR3 region having SEQ ID NO:307, and/or
a CDR1 region having SEQ ID NO:141, a CDR2 region having has SEQ ID NO:225, and a CDR3 region having SEQ ID NO:308, and/or
a CDR1 region having SEQ ID NO:142, a CDR2 region having has SEQ ID NO:226, and a CDR3 region having SEQ ID NO:309, and/or
a CDR1 region having SEQ ID NO:143, a CDR2 region having has SEQ ID NO:227, and a CDR3 region having SEQ ID NO:310, and/or
a CDR1 region having SEQ ID NO:144, a CDR2 region having has SEQ ID NO:228, and a CDR3 region having SEQ ID NO:311, and/or
a CDR1 region having SEQ ID NO:145, a CDR2 region having has SEQ ID NO:229, and a CDR3 region having SEQ ID NO:312, and/or
a CDR1 region having SEQ ID NO:146, a CDR2 region having has SEQ ID NO:230, and a CDR3 region having SEQ ID NO:313, and/or
a CDR1 region having SEQ ID NO:147, a CDR2 region having has SEQ ID NO:231, and a CDR3 region having SEQ ID NO:314, and/or
a CDR1 region having SEQ ID NO:148, a CDR2 region having has SEQ ID NO:232, and a CDR3 region having SEQ ID NO:315, and/or
a CDR1 region having SEQ ID NO:149, a CDR2 region having has SEQ ID NO:233, and a CDR3 region having SEQ ID NO:316, and/or
a CDR1 region having SEQ ID NO:150, a CDR2 region having has SEQ ID NO:234, and a CDR3 region having SEQ ID NO:317, and/or
a CDR1 region having SEQ ID NO:151, a CDR2 region having has SEQ ID NO:235, and a CDR3 region having SEQ ID NO:318, and/or
a CDR1 region having SEQ ID NO:152, a CDR2 region having has SEQ ID NO:236, and a CDR3 region having SEQ ID NO:319, and/or
a CDR1 region having SEQ ID NO:153, a CDR2 region having has SEQ ID NO:237, and a CDR3 region having SEQ ID NO:320, and/or
a CDR1 region having SEQ ID NO:154, a CDR2 region having has SEQ ID NO:238, and a CDR3 region having SEQ ID NO:321, and/or
a CDR1 region having SEQ ID NO:155, a CDR2 region having has SEQ ID NO:239, and a CDR3 region having SEQ ID NO:322, and/or
a CDR1 region having SEQ ID NO:156, a CDR2 region having has SEQ ID NO:240, and a CDR3 region having SEQ ID NO:323, and/or
a CDR1 region having SEQ ID NO:157, a CDR2 region having has SEQ ID NO:241, and a CDR3 region having SEQ ID NO:324, and/or
a CDR1 region having SEQ ID NO:158, a CDR2 region having has SEQ ID NO:242, and a CDR3 region having SEQ ID NO:325, and/or
a CDR1 region having SEQ ID NO:159, a CDR2 region having has SEQ ID NO:243, and a CDR3 region having SEQ ID NO:326, and/or
a CDR1 region having SEQ ID NO:160, a CDR2 region having has SEQ ID NO:244, and a CDR3 region having SEQ ID NO:327, and/or
a CDR1 region having SEQ ID NO:161, a CDR2 region having has SEQ ID NO:245, and a CDR3 region having SEQ ID NO:328, and/or
a CDR1 region having SEQ ID NO:162, a CDR2 region having has SEQ ID NO:246, and a CDR3 region having SEQ ID NO:329, and/or
a CDR1 region having SEQ ID NO:163, a CDR2 region having has SEQ ID NO:247, and a CDR3 region having SEQ ID NO:330, and/or
a CDR1 region having SEQ ID NO:164, a CDR2 region having has SEQ ID NO:248, and a CDR3 region having SEQ ID NO:331, and/or
a CDR1 region having SEQ ID NO:165, a CDR2 region having has SEQ ID NO:249, and a CDR3 region having SEQ ID NO:332, and/or
a CDR1 region having SEQ ID NO:166, a CDR2 region having has SEQ ID NO:250, and a CDR3 region having SEQ ID NO:333, and/or
a CDR1 region having SEQ ID NO:167, a CDR2 region having has SEQ ID NO:251, and a CDR3 region having SEQ ID NO:334, and/or
a CDR1 region having SEQ ID NO:168, a CDR2 region having has SEQ ID NO:252, and a CDR3 region having SEQ ID NO:335.

In certain embodiments of the transgenic plants or plant tissues or plant cells, methods, or uses, as taught herein, the polynucleotide may comprise a sequence encoding a VHH comprising a CDR1, CDR2 and CDR3 region chosen from the list of comprising:
a CDR1 region having SEQ ID NO:85, a CDR2 region having has SEQ ID NO:169, and a CDR3 region having SEQ ID NO:253, and/or
a CDR1 region having SEQ ID NO:86, a CDR2 region having has SEQ ID NO:170, and a CDR3 region having SEQ ID NO:254.

In certain embodiments of the transgenic plants or plant tissues or plant cells, methods, or uses, as taught herein, the polynucleotide may comprise a sequence encoding a VHH comprising a CDR1 region having SEQ ID NO:85, a CDR2 region having has SEQ ID NO:169, and a CDR3 region having SEQ ID NO:253.

In certain embodiments of the transgenic plants or plant tissues or plant cells, methods, or uses, as taught herein, the polynucleotide may comprise a sequence encoding a VHH comprising a CDR1 region having SEQ ID NO:86, a CDR2 region having has SEQ ID NO:170, and a CDR3 region having SEQ ID NO:254.

In particular embodiments, the VHH as taught herein are heavy chain variable domains that essentially consist of four framework regions (FR1 to FR4, respectively) and three complementarity-determining regions (CDR1 to CDR3, respectively); or any suitable fragment of such an heavy chain variable domain (which will then usually contain at least some of the amino acid residues that form at least one of the CDRs, as further described herein).

Functional variants of the VHH as taught herein may, in particular, be a domain antibody (or an heavy chain variable domain that is suitable for use as a domain antibody), a single domain antibody (or an heavy chain variable domain that is suitable for use as a single domain antibody), or a “dAb” (or an heavy chain variable domain that is suitable for use as a dAb); other single variable domains, or any suitable fragment of any one thereof. For a general description of (single) domain antibodies, reference is also made to the prior art cited above, as well as to EP 0 368 684. For the term “dAbs,” reference is made, for example, to Ward et al. (Nature 1989 Oct. 12; 341 (6242):544-6), to Holt et al., Trends Biotechnol. 2003, 21 (11):484-490; as well as to, for example, WO 06/030220, WO 06/003388 and other published patent applications of Domantis Ltd.

Thus, in particular embodiments, this disclosure provides a variable domain of a heavy-chain antibody with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity-determining regions 1 to 3, respectively, and are as further defined herein.

SEQ ID NOS:1 to 84 (see Table 1) give the amino acid sequences of a number of variable domains of a heavy-chain antibody that have been raised against a sphingolipid target, in particular, against glucosylceramide.

TABLE 1
VHH sequences

| Name | SEQ ID | VHH Amino acid sequence |
|---|---|---|
| 41D01 | 1 | QVQLQESGGGLVQAGGSLRLSCAASGRTFSRYGMGWFRQLPGKQRELVTSIT |
| | | RGGTTTYADSVKGRFTISRDNAKNTVYLQMNSLKPEDTAVYYCNARSIWRDY |
| | | WGQGTQVTVSS |
| 56F11 | 2 | QVQLQESGGGLVQSGGSLRLSCVHSKTTFTRNAMGWYRQALGKERELVATIT |
| | | SGGTTNYADSVKGRFTISMDSAKNTVYLQMNSLKPEDTAVYYCNVNTRRIFG |
| | | GTVREYWGQGTQVTVSS |
| 40F07 | 3 | QVQLQESGGGLVQAGGSLRLSCVASGTTFSSYTMGWYRQAPGKQRELLASIE |
| | | GGGNTDYADSVKGRFTISRDNARNTVYLQMNSLKTEDTAVYYCNAARTWSIF |
| | | RNYWGQGTQVTVSS |
| 41D06 | 4 | QVQLQESGGGLVQAGGSLRLSCAASGGIFGINAMRWYRQAPGKQRELVASISS |
| | | GGNTNYSESVKGRFTISRDDANYTVYLQMNSLKPEDTAVYYCNFVRLWFPDY |
| | | WGQGTQVTVSS |
| 41G10 | 5 | QVQLQESGGGLVQPGGSLTLSCAATKTGFSINAMGWYRQAPGKQREMVATIT |
| | | SGGTTNYADSVKGRFAISRDNAKNTVSLQMNTLKPEDTALYYCNTEARRYFT |
| | | RASQVYWGQGTQVTVSS |
| 41H05 | 6 | QVQLQESGGGLVQPGGSLRLSCAASGGIFSINAMGWYRQDPGKQREMVATITS |
| | | GANTNYTDSVKGRFTISRDNAKNTVYLQMNSLKPEDTAVYYCNAVGRRWYG |
| | | GYVELWGQGTQVTVSS |
| 42C11 | 7 | QVQLQESGGGLVQPGGSLRLSCAASGSIFSTYVMGWYRQAIGKQRELVATITS |
| | | SGKTNYAASVKGRFTVSRDITKNTMYLQMNSLKPEDTAVYYCGADRWVLTR |
| | | WSNYWGQGTQVTVSS |
| 42C12 | 8 | QVQLQESGGGLVQPGGSLRLSCAASGSISSLGWYRQAPGKQREFVASATSGGD |
| | | TTYADSVKGRFTISRDNSKNTVYLQMNSLKPEDTAVYYCKGQRGVAWTRKE |
| | | YWGQGTQVTVSS |
| 50D03 | 9 | QVQLQESGGGLVQPGGSLRLSCAASGSIFSTYAMGWYRQAIGKQRELVATITS |
| | | SGKTNYAASVKGRFTISRDITKNTMYLQMNSLKPEDTAVYYCGADRWVLTR |
| | | WSNYWGQGTQVTVSS |
| 50D07 | 10 | QVQLQESGGGLVQPGGSLRLSCTASGNIVNIRDMGWYRQVPGKQRELVATITS |
| | | DQSTNYADSVKGRFTTTRDNAKKTVYLQMDSLKPEDTAGYYCNARVRTVLR |
| | | GWRDYWGQGTQVTVSS |
| 50E02 | 11 | QVQLQESGGGLVQPGGSLRLSCAASGSIFSINAMGWYRQAPGKQRELVAAITS |
| | | DGSTNYADSVKGRFTISRDNAKNTAYLQMNSLKPEDTAVYYCNLRRRTFLKSS |
| | | DYWGQGTQVTVSS |
| 51B08 | 12 | QVQLQESGGGLVQAGDSLRLSCAASGRRFGSYAMGWFRQVPGKERELVAGIS |
| | | SGGSTKYADSVRGRFTISRDNAKNTVSLQMKSLKPEDTAVYYCNAKYGRWTY |
| | | TGRPEYDSWGQGTQVTVSS |
| 51C06 | 13 | QVQLQESGGGLVQPGGSLRLSCAASGSIFSSDTMGWYRRAPGKQRELVAAITT |
| | | GGNTNYADSVKGRFTISRDNAKNTVYLQMNSLQPEDTAVYYCNCRRRWSRD |
| | | FWGQGTQVTVSS |
| 51C08 | 14 | QVQLQESGGGLVQPGGSLRLSCAASGTIFSIKTMGWYRQAPGKQRELVATISN |
| | | GGSTNYADSVKGRFTISRDNAKNTVYLQMNSLKPEDTAVYYCNARQQFIGAP |
| | | YEYWGQGTQVTVSS |
| 52A01 | 15 | QVQLQESGGGLVQAGGSLRLSCTASGAITFSLGTMGWYRQAPGKQRELVASIS |
| | | TGSTNYADSVKGRFTISRDIIKNILYLQMNSLKPEDTAVYSCNARLLWSNYWG |
| | | QGTQVTVSS |
| 52B01 | 16 | QVQLQESGGGLVQAGESLRLSCAASGSTFSINVMGWYRQAPGEQRELVATISR |
| | | GGSTNYADSVKGRFTISRDNAKVTVYLQMDSLKPEDTAVYYCNAAGWVGVT |
| | | NYWGQGTQVTVSS |
| 52G05 | 17 | QVQLQESGGGLVQAGGSLRLSCAASGSTGSISAMGWYRQAPGKQRELVASITR |
| | | RGSTNYADSVKDRFTISRDNAWNTVYLQMNSLKPEDTAVYYCNARRYYTRN |
| | | DYWGQGTQVTVSS |
| 53A01 | 18 | QVQLQESGGGLGQAGGSLRLSCEVSGTTFSINTMGWHRQAPGKQRELVASISS |
| | | GGWTNYADSVKGRFTISRDNAKKTVYLQMNNLKPEDTAVYYCRWGAIGNW |
| | | YGQGTQVTVSS |
| 53F05 | 19 | QVQLQESGGGLVQPGGSLRLSCAASVRIFGLNAMGWYRQGPGKQRELVASIT |
| | | TGGSTNYAEPVKGRFTISRDNANNTVYLQMNNLKPEDTAVYYCNAERRWGLP |
| | | NYWGQGTQVTVSS |
| 54A02 | 20 | QVQLQESGGGLVEAGGSLRLSCAASGRTFSRYGMGWFRQAPGKEREFVAANR |
| | | WSGGSTYYADSVRGRFTISRDNAKNTVYLQMNSLKPEDTAVYYCAAYAHITA |
| | | WGMRNDYEYDYWGQGTQVTVSS |
| 54B01 | 21 | QVQLQESGGGLVQAGGSLRLSCAATGRTFSRYTMGWFRQAPGKERDFVAGIT |
| | | WTGGSTDYADSVKGRFTISRDNAKNTVYLQMNSLKPEDTAVYYCAAGNLLRL |
| | | AGQLRRGYDSWGQGTQVTVSS |
| 54C01 | 22 | QVQLQESGGGLVQAGGSLRLSCAASGRTGSRYAMGWFRQAPGKEREFVAAIS |
| | | WSGGSTYYADSVKDRFTISRDNAKNTVYLQMHSLKPEDTAVYYCATRNRAGP |
| | | HYSRGYTAGQEYDYWGQGTQVTVSS |
| 54C04 | 23 | QVQLQESGGGLVQPGGSLRLSCAASGRIFSINAMGWYRQGPGKERELVVDMT |
| | | SGGSINYADSVSGRFTISRDNAKNTVYLQMNSLKPEDTAVYYCHANLRTAFW |
| | | RNGNDYWGQGTQVTVSS |
| 54C08 | 24 | QVQLQESGGGLVQPGGSLRLSCAASGSISSINAMGWYRQAPGKQRELVASITS |
| | | GGSTNYADSVKGRFTISRDNAKNTVNLQMNSLKPEDTAVYYCSAGPWYRRS |
| | | WGRGTQVTVSS |
| 54C10 | 25 | QVQLQESGGGLVQPGESLRLSCAASASIFWVNDMGWYRQAPGKQRELVAQIT |
| | | RRGSTNYADSVKGRFTISRDNAKDEVYLQMNSLKPEDTAVYYCNADLAVRGR |
| | | YWGQGTQVTVSS |
| 54C11 | 26 | QVQLQESGGGLVQPGGSLRLSCAASGSFFPVNDMAWYRQALGNERELVANIT |
| | | RGGSTNYADSVKGRFTISRDNAKNTVYLQMNTLKPEDTAVYYCNVRIGFGWT |
| | | AKAYWGQGTQVTVSS |
| 54D03 | 27 | QVQLQESGGGLVQPGGSLRLSCAASGGIFGINAMRWYRQAPGKQRELVASISS |
| | | GGNTNYSESVKGRFTISRDDANYTVYLQMNSLKPEDTAVYYCNFVRLWFPDY |
| | | WGQGTQVTVSS |
| 54D06 | 28 | QVQLQESGGGLVQPGGSLRLSCAASGSTIRINAMGWYRQAPGKQRELVATITR |
| | | GGITNYADSVKGRFTISRDNAKFTVYLQMNSLKPEDTAVYYCNARSWVGPEY |
| | | WGQGTQVTVSS |
| 54D10 | 29 | QVQLQESGGGLVQPGGSLRLSCAASGMTYSIHAMGWYRQAPGKERELVAITS |
| | | TSGTTDYTDSVKGRFTISRDGANNTVYLQMNSLKSEDTAVYYCHVKTRTWYN |
| | | GKYDYWGQGTQVTVSS |
| 54E01 | 30 | QVQLQESGGGLVQPGGSLRLSCTASGSIFSINPMGWYRQAPGKQRELVAAITS |
| | | GGSTNYADYVKGRFTISRDNAKNVVYLQMNSLKPEDTAVYYCNGRSTLWRR |
| | | DYWGQGTQVTVSS |
| 54E05 | 31 | QVQLQESGGGLVQPGGSLRLSCAASGSIFSINTMGWYRQAPGKQRELVAAITN |
| | | RGSTNYADFVKGRFTISRDNAKNTVYLQMNSLKPDDTAVYYCNAHRSWPRY |
| | | DSWGQGTQVTVSS |
| 54E10 | 32 | QVQLQESGGGLVQPGGSLRLSCAASGSIFSFNAMGWYRQAPGKQRELVAAITR |
| | | GGSTNYADSVKGRFTISRDNANNTVYLQMNSLKPEDTAVYYCNAESRIFRRYD |
| | | YWGPGTQVTVSS |
| 54F01 | 33 | QVQLQESGGGLVQPGGSLRLSCVTSGSIFGLNLMGWYRQAPGKQRELVATITR |
| | | GGSTNYADSVKGRFTISRDNAKKTVYLQMNSLKPEDTAVYYCNVDRGWSSY |
| | | WGQGTQVTVSS |
| 54F02 | 34 | QVQLQESGGGLVQPGGSLRLSCVTSGSIRSINTMGWYRQAPGNERELVATITSG |
| | | GTTNYADSVKNRFTISRDNAKNTVYLQMNSLKPEDTAVYYCNLHQRAWARS |
| | | YVYWGQGTQVTVSS |
| 54G01 | 35 | QVQLQESGGGSVQPGGSLRLSCAASGSIFAVNAMGWYRQAPGHQRELVAIISS |
| | | NSTSNYADSVKGRFTISRDNAKNTVYLQMNSLKPEDTAVYFCYAKRSWFSQE |
| | | YWGQGTQVTVSS |
| 54G08 | 36 | QVQLQESGGGLVQPGGSLRLSCAASGSIFSFNLMGWYRQAPGKQRELVAAITS |
| | | SSNTNYADSVKGRFTISRDNAKNTVYLQMNSLKPEDTAVYYCNAQYTITPWGI |
| | | KKDYWGQGTQVTVSS |
| 54G09 | 37 | QVQLQESGGGLMQPGGSLRLSCTASGNIVNIRDMGWYRQVPGKQRELVATITS |
| | | DQSTNYADSVKGRFTTTRDNAKKTVYLQMDSLKPEDTAGYYCNARVRTVLR |
| | | GWRDYWGQGTQVTVSS |
| 55B02 | 38 | QVQLQESGGGLVQPGESLRLSCVGSGSIFNINSMNWYRQASGKQRELVADMR |
| | | SDGSTNYADSVKGRFTISRDNARKTVYLQMNSLKPEDTAVYYCHANSIFRSRD |
| | | YWGQGTQVTVSS |
| 55B05 | 39 | QVQLQESGGGVVQAGDSLRLSCAASGRTFGGYTVAWFRQAPGKEREFVARIS |
| | | WSGIMAYYAESVKGRFTISRDNAKNTVYLQMNSLKPEDTAVYYCASRSQIRSP |
| | | WSSLDDYDRWGQGTQVTVSS |
| 55C05 | 40 | QVQLQESGGGLVQPGGSLRLSCVVSGSISSMKAMGWHRQAPGKERELVAQIT |
| | | RGDSTNYADSVKGRFTISRDNAKNTVYLQMNSLKPDDTGVYYCNADRFFGRD |
| | | YWGKGTQVTVSS |
| 55D08 | 41 | QVQLQESGGGLVQPGGSLRLSCAASRSILSISAMGWYRQGPGKQREPVATITSA |
| | | GSSNYSDSVKGRFTISRDNAKNTAYLQMNSLKPEDTAVYYCKTVYSRPLLGPL |
| | | EVWGQGTQVTVSS |
| 55E02 | 42 | QVQLQESGGGLVQTGGSLRLSCVASGSMFSSNAMAWYRQAPGKQRELVARIL |
| | | SGGSTNYADSVKGRFTISRGNAKNTVYLQMNSLKPEDTAVYYCNAVRYLVNY |
| | | WGQGTQVTVSS |
| 55E07 | 43 | QVQLQESGGGSVQVGDSLTLSCVASGRSLDIYGMGWFRQAPGKEREFVARITS |
| | | GGSTYYADSVKGRFTLSRDNAKNTVYLQMNSLKPEDTAVYYCAAGVVVATS |
| | | PKFYAYWGQGTQVTVSS |
| 55E09 | 44 | QVQLQESGGGLVQAGGSLRLSCAASKRIFSTYTMGWFRQAPGKEREFVAAIIW |
| | | SGGRTRYADSVKGRFTISRDNARNTVHLQMNSLEPEDTAVYYCYTRRLGTGY |
| | | WGQGTQVTVSS |
| 55E10 | 45 | QVQLQESGGGLVQAGGSLRLSCAASGSTFSIQTIGWYRQAPGKQRDRVATISS |
| | | GGSTNYADSVKGRFTISRDNAKKTVYLQMNNLKPEDTAVYYCNLRYWFRDY |
| | | WGQGTQVTVSS |
| 55F04 | 46 | QVQLQESGGGLVQPGGSLRLSCAASGSTFSINVRGWYRQAPGKQRELVATITS |
| | | DGSTNYADSVKGRFTISRDNAKNTAYLQMNSLKPEDTAVYYCNAVRLFRQY |
| | | WGQGTQVTVSS |
| 55F09 | 47 | QVQLQESGGGLVQPGGSLRLSCAASGSIFRLNAMGWYRQAPGKQRELVAAITP |
| | | GGGNTTYADSVKGRFTISRDNALNTIYLQMNSLKPEDTAVYYCNAGGSSRWY |
| | | SSRYYPGGYWGQGTQVTVSS |
| 55F10 | 48 | QVQLQESGGGLVQAGGSLRLSCATSGGTFSRYAMGWFRQAPGKERELVATIR |
| | | RSGSSTYYLDSTKGRFTISRDNAKNTVYLQMNSLKLEDTAVYYCAADSSARAL |
| | | VGGPGNRWDYWGQGTQVTVSS |
| 55G02 | 49 | QVQLQESGGGLVQPGGSLRLSCAASGSIGSINVMGWYRQYPGKQRELVAFITS |
| | | GGITNYTDSVKGRFAISRDNAQNTVYLQMNSLTPEDTAVYYCHLKNAKNVRP |
| | | GYWGQGTQVTVSS |
| 55G08 | 50 | QVQLQESGGGLVQPGGSLRLSCRASGGIFGINAMRWYRQAPGKQRELVASISS |
| | | GGTTDYVESVKGRFTISRDNATNTVDLQMSALKPEDTAVYYCNFVRFWFPDY |
| | | WGQGTQVTVSS |
| 56A05 | 51 | QVQLQESGGGLVQAGGSLRLSCAASGITFMSNTMGWYRQAPGKQRELVASIS |
| | | SGGSTNYADSVKGRFTISRDNAKKTVYLQMNSLKPEDTAVYYCNARRNVFISS |
| | | WGQGTQVTVSS |
| 56A06 | 52 | QVQLQESGGGLVQPGGSLRLSCVASGSISVYGMGWYRQAPGKQRELVARITNI |
| | | GTTNYADSVKGRFTISRDNAKNTVYLQMNSLQPEDTAVYYCNLRRLGRDYW |
| | | GQGTQVTVSS |
| 56A09 | 53 | QVQLQESGGGLVQPGGSLRLSCAASRTALRLNSMGWYRQAPGSQRELVATIT |
| | | RGGTTNYADSVKGRFTISREIGNNTVYLQMNSLEPEDTAVYYCNANFGILVGR |
| | | EYWGKGTQVTVSS |
| 56C09 | 54 | QVQLQESGGGLVQAGGSLRLSCAVSGSIFSILSMAWYRQTPGKQRELVANITS |
| | | VGSTNYADSVKGRFTISRDIAKKTLYLQMNNLKPEDTAIYYCNTRMPFLGDSW |
| | | GQGTQVTVSS |
| 56C12 | 55 | QVQLQESGGGLVQAGGSLRLSCAVSAFSFSNRAVSWYRQAPGKSREWVASIS |
| | | GIRITTYTNSVKGRFIISRDNAKKTVYLQMNDLRPEDTGVYRCYMNRYSGQGT |
| | | QVTVSS |
| 56D06 | 56 | QVQLQESGGGSVQPGGSLRLSCAASGTVFFSISAMGWYRQAPGKQRELVAGIS |
| | | RGGSTKYGDFVKGRFTISRDNGKKTIWLQMNNLQPEDTAIYYCRLTSITGTYL |
| | | WGQGTQVTVSS |
| 56D07 | 57 | QVQLQESGGGLVQPGGSLRLSCAASGSIFSMKVMGWYRQGPGKLRELVAVIT |
| | | SGGRTNYAESVKGRFTISRDNAKNTVSLQMNSLQPEDTAVYYCYYKTIRPYW |
| | | GQGTQVTVSS |
| 56D10 | 58 | QVQLQESGGGLVQAGGSLRLSCAASGITFRITTMGWYRQAPGKQRELVASSSS |
| | | GGTTNYASSVKGRFTISRDNAKNTVYLQMNSLRPEDTAVYYCNARKFITTPWS |
| | | TDYWGQGTQVTVSS |
| 56E04 | 59 | QVQLQESGGGLVQPGDSLRLSCTPSGSIFNHKATGWYRQAPGSQRELVAKITT |
| | | GGTTNYADSVKGRFTISRDNAKNTVYLQMSSLKPEDTAVYYCNAERYFATTL |
| | | WGQGTQVTVSS |
| 56E05 | 60 | QVQLQESGGGLVQAGGSLRLSCAASGITFSNNAGGWYRQAPGQQRELVARISS |
| | | GGNTNYTDSVKGRFTISRDITKNTLSLQMNNLKPEDSAVYYCNAQRRVILGPR |
| | | NYWGQGTQVTVSS |
| 56E08 | 61 | QVQLQESGGGLVQAGGSLRLSCAASGNIFRINDMGWYRQAPGNQRELVATITS |
| | | ANITNYADSVKGRFTISRDNAKNTVYLQMNSLNPEDTAVYYCTAQAKKWRIG |
| | | PWSDYWGQGTQVTVSS |
| 56F07 | 62 | QVQLQESGGGLVQPGGSLRLSCAASGRIFSINDMAWYRQAPGKQRELVAIITN |
| | | DDSTTYADSVKGRFTISRDNAKNTVYLQMNSLKPEDTAVYYCNADINTAIWR |
| | | RKYWGQGTQVTVSS |
| 56G07 | 63 | QVQLQESGGGLVQPGGSLRLSCAVSGSRIFIHDMGWHRQAPGEPRELVATITPF |
| | | GRRNYSEYVKGRFTVSRDIARNTMSLQMSNLKAEDTGMYYCNVRVNGVDY |
| | | WGQGTQVTVSS |
| 56G08 | 64 | QVQLQESGGGLVQAGGSLRLSCAISGITFRRPFGISRMGWYRQAPGKERELVA |
| | | TLSRAGTSRYVDSVKGRFTISRDDAKNTLYLQMVSLNPEDTAVYYCYIAQLGT |
| | | DYWGQGTQVTVSS |
| 56G10 | 65 | QVQLQESGGGLVQAGGSLRLSCVASGITLRMYQVGWYRQAPGKQRELVAEIS |
| | | SRGTTMYADSVKGRFTISRDGAKNIVYLQMNSLEPEDTAVYYCNARAFAFGR |
| | | NSWGQGTQVTVSS |
| 56H04 | 66 | QVQLQESGGGSVQAGGSLRLSCAVSGGTFSNKAMGWYRQSSGKQRALVARIS |
| | | TVGTAHYADSVKGRFTVSKDNAGNTLYLQMNSLKPEDTAVYYCNAQAGRLY |
| | | LRNYWGQGTQVTVSS |
| 56H05 | 67 | QVQLQESGGGLVQPGESLRLSCVAAASTSITTFNTMAWYRQAPGKQRELVAQI |
| | | NNRDNTEYADSVKGRFIISRGNAKNTSNLQMNDLKSEDTGIYYCNAKRWSWS |
| | | TGFWGQGTQVTVSS |
| 56H07 | 68 | QVQLQESGGGLVQAGGSLRLSCTASGLTFALGTMGWYRQAPGKQRELVASIS |
| | | TGSTNYADSVKGRFTISRDIIKNILYLQMNSLKPEDTAVYSCNARLWWSNYWG |
| | | QGTQVTVSS |
| 56H08 | 69 | QVQLQESGGGLVQAGGSLRLSCTASGRTSSVNPMGWYRQAPGKQRELVAVIS |
| | | SDGSTNYADSVKGRFTVSRDNAKNTLYLQMNSLKPEDTAVYYCNANRRWSW |
| | | GSEYWGQGTQVTVSS |
| 57A06 | 70 | QVQLQESGGGLVQAGGSLRLSCAASGITFTNNAGGWYRQAPGQQRELVARISS |
| | | GGNTNYTDSVKGRFTISRDITKNTLSLQMNNLKPEDSAVYYCNAQRRVILGPR |
| | | NYWGQGTQVTVSS |
| 57B01 | 71 | QVQLQESGGGLVQAGGSLRLSCEAPVSTFNINAMAWYRQAPGKSRELVARISS |
| | | GGSTNYADSVKGRFTISRDNAKNTVYLQMNSLKPEDTAVYICYVNRHWGWD |
| | | YWGQGTQVTVSS |
| 57B07 | 72 | QVQLQESGGGLVQPGGTLRLSCVASGSFRSINAMGWYRQAPGKQRELVATVD |
| | | SGGYTNYADSVKGRFTISRDNAKNTVYLQMSSLTPEDTAVYYCYAGIYKWPW |
| | | SVDARDYWGQGTQVTVSS |
| 57B11 | 73 | QVQLQESGGGLVQAGGSLRLSCAASGSSISMNSMGWYRQAPGKERERVALIR |
| | | SSGGTYYADSVKGRFTISRDNAKNTVYLQMNNLKPEDTAVYYCQARRTWLSS |
| | | ESWGQGTQVTVSS |
| 57C07 | 74 | QVQLQESGGGLVQAGGSLRLSCAVSGSTFGINTMGWYRQAPEKQRELVASISR |
| | | GGMTNYADSVKGRFIISRDNAKNTVYLQMNSLKPEDTAVYVCNAGIRSRWYG |
| | | GPITTYWGQGTQVTVSS |
| 57C09 | 75 | QVQLQESGGGLVQAGGSLRLSCAASGSTGSINAMGWYRQGPGKQRDLVASIS |
| | | SGGATNYADSVKGRFTISRDNSKNTVYLQMSSLKPEDTAVYYCNAKKSRWSW |
| | | SIVHDYWGQGTQVTVSS |
| 57D02 | 76 | QVQLQESGGGSVQTGGSLTLSCTTSGSIFGRSDMGWYRQAPGKQRELVATITR |
| | | RSRTNYAEFVKGRFTISRDSAKNLVTLQMNSLKPEDTNVYYCNARWGAGGIFS |
| | | TWGQGTQVTVSS |
| 57D09 | 77 | QVQLQESGGGLVQPGESLRLSCAASGSMSIDAMGWYRQAPGDQRELVASITT |
| | | GGSTNYADSVKGRFTISRDNAKNTVWLQMNSLKPEDTAVYYCNAKVRLRWF |
| | | RPPSDYWGQGTQVTVSS |
| 57D10 | 78 | QVQLQESGGGLVQPGGSLRLSCAASGRLLSISTMGWYRRTPEDQREMVASITK |
| | | DGTTNYADSVKGRLTISRDNAKNTVYLQMNSLKPDDTAVYVCNARATTWVP |
| | | YRRDAEFWGQGTQVTVSS |
| 57E07 | 79 | QVQLQESGGGLVQAGGSLRLSCAASGSIFGINDMGWYRQAPGKQRDLVADIT |
| | | RSGSTHYVDSVKGRFTISRDNAKNTVYLQMNSLKPEDTAVYYCNADSGSHW |
| | | WNRRDYWGQGTQVTVSS |
| 57E11 | 80 | QVQLQESGGGLVQPGGSLKLSCAASGFTFSINTMGWYRQAPGKQRELVARISR |
| | | LRVTNYADSVKGRFTISRDNAKNTVYLQMNSLKPEDTAVYYCNAANWGLAG |
| | | NEYWGQGTQVTVSS |
| 57G01 | 81 | QVQLQESGGGLVQAGGSLRPSCTASGSTLLINSMGWYRQAPGKQRELVATISN |
| | | SGTTNYVDAVKGRFAISRDNANHTVYLQMNSLEPEDTAVYYCNAQTFWRRN |
| | | YWGQGTQVTVSS |
| 57G07 | 82 | QVQLQESGGGLVQAGGSLRLSCAVSGSTSRINAMGWYRQAPGKKRESVATIR |
| | | RGGNTKYADSVKGRFTISRDNANNTVYLQLNSLKPEDTAVYYCNAHSWLDY |
| | | DYWGRGTQVTVSS |
| 57G08 | 83 | QVQLQESGGGLVQAGGSLRLSCASRRRINGITMGWYRQAPGKQRELVATIDIH |
| | | NSTKYADSVKGRFIISRDNGKSMLYLQMNSLKPEDTAVYYCNRIPTFGRYWGQ |
| | | GTQVTVSS |
| 57H08 | 84 | QVQLQESGGGLVQAGGSLRLSCVASGSTFYTFSTKNVGWYRQAPGKQRELVA |
| | | QQRYDGSTNYADSLQGRFTISRDNAKRTVYLQMNSLKPEDTAVYICNVNRGFI |
| | | SYWGQGTQVTVSS |

In particular, the disclosure in some specific embodiments provides transgenic plants or plant tissues or plant cells comprising a polynucleotide encoding at least one VHH that is directed against a sphingolipid target and that has at least 80%, preferably at least 85%, such as 90% or 95% or more sequence identity with at least one of the heavy chain variable domains of SEQ ID NOS:1 to 84 (see Table 1), and nucleic acid sequences that encode such heavy chain variable domains.

Some particularly preferred VHH as disclosed herein are those that can bind to and/or are directed against a sphingolipid of a plant pathogen and that have at least 90% amino acid identity with at least one of the VHH of SEQ ID NOS:1 to 84 (see Table 1), in which, for the purposes of determining the degree of amino acid identity, the amino acid residues that form the CDR sequences are disregarded.

In these heavy chain variable domains, the CDR sequences (see Table 2) are generally as further defined herein.

TABLE 2
CDR sequences

| | CDR1 | | | | | |
|---|---|---|---|---|---|---|
| Name | sequence | SEQ ID | CDR2 sequence | SEQ ID | CDR3 sequence | SEQ ID |
| 41D01 | RYGMG | 85 | SITRGGTTTYADSVKG | 169 | RSIWRDY | 253 |
| 56F11 | RNAMG | 86 | TITSGGTTNYADSVKG | 170 | NTRRIFGGTVREY | 254 |
| 40F07 | SYTMG | 87 | SIEGGGNTDYADSVKG | 171 | ARTWSIFRNY | 255 |
| 41D06 | INAMR | 88 | SISSGGNTNYSESVKG | 172 | VRLWFPDY | 256 |
| 41G10 | INAMG | 89 | TITSGGTTNYADSVKG | 173 | EARRYFTRASQVY | 257 |
| 41H05 | INAMG | 90 | TITSGANTNYTDSVKG | 174 | VGRRWYGGYVEL | 258 |
| 42C11 | TYVMG | 91 | TITSSGKTNYAASVKG | 175 | DRWVLTRWSNY | 259 |
| 42C12 | ISSLG | 92 | SATSGGDTTYADSVKG | 176 | QRGVAWTRKEY | 260 |
| 50D03 | TYAMG | 93 | TITSSGKTNYAASVKG | 177 | DRWVLTRWSNY | 261 |
| 50D07 | IRDMG | 94 | TITSDQSTNYADSVKG | 178 | RVRTVLRGWRDY | 262 |
| 50E02 | INAMG | 95 | AITSDGSTNYADSVKG | 179 | RRRTFLKSSDY | 263 |
| 51B08 | SYAMG | 96 | GISSGGSTKYADSVRG | 180 | KYGRWTYTGRPEY | 264 |
| | | | | | DS | |
| 51C06 | SDTMG | 97 | AITTGGNTNYADSVKG | 181 | RRRWSRDF | 265 |
| 51C08 | IKTMG | 98 | TISNGGSTNYADSVKG | 182 | RQQFIGAPYEY | 266 |
| 52A01 | LGTMG | 99 | SISTGSTNYADSVKG | 183 | RLLWSNY | 267 |
| 52B01 | INVMG | 100 | TISRGGSTNYADSVKG | 184 | AGWVGVTNY | 268 |
| 52G05 | ISAMG | 101 | SITRRGSTNYADSVKD | 185 | RRYYTRNDY | 269 |
| 53A01 | INTMG | 102 | SISSGGWTNYADSVKG | 186 | GAIGNW | 270 |
| 53F05 | LNAMG | 103 | SITTGGSTNYAEPVKG | 187 | ERRWGLPNY | 271 |
| 54A02 | RYGMG | 104 | ANRWSGGSTYYADSVRG | 188 | YAHITAWGMRND | 272 |
| | | | | | YEYDY | |
| 54B01 | RYTMG | 105 | GITWTGGSTDYADSVKG | 189 | GNLLRLAGQLRRG | 273 |
| | | | | | YDS | |
| 54C01 | RYAMG | 106 | AISWSGGSTYYADSVKD | 190 | RNRAGPHYSRGYT | 274 |
| | | | | | AGQEYDY | |
| 54C04 | INAMG | 107 | DMTSGGSINYADSVSG | 191 | NLRTAFWRNGNDY | 275 |
| 54C08 | INAMG | 108 | SITSGGSTNYADSVKG | 192 | GPWYRRS | 276 |
| 54C10 | VNDMG | 109 | QITRRGSTNYADSVKG | 193 | DLAVRGRY | 277 |
| 54C11 | VNDMA | 110 | NITRGGSTNYADSVKG | 194 | RIGFGWTAKAY | 278 |
| 54D03 | INAMR | 111 | SISSGGNTNYSESVKG | 195 | VRLWFPDY | 279 |
| 54D06 | INAMG | 112 | TITRGGITNYADSVKG | 196 | RSWVGPEY | 280 |
| 54D10 | IHAMG | 113 | ITSTSGTTDYTDSVKG | 197 | KTRTWYNGKYDY | 281 |
| 54E01 | INPMG | 114 | AITSGGSTNYADYVKG | 198 | RSTLWRRDY | 282 |
| 54E05 | INTMG | 115 | AITNRGSTNYADFVKG | 199 | HRSWPRYDS | 283 |
| 54E10 | FNAMG | 116 | AITRGGSTNYADSVKG | 200 | ESRIFRRYDY | 284 |
| 54F01 | LNLMG | 117 | TITRGGSTNYADSVKG | 201 | DRGWSSY | 285 |
| 54F02 | INTMG | 118 | TITSGGTTNYADSVKN | 202 | HQRAWARSYVY | 286 |
| 54G01 | VNAMG | 119 | IISSNSTSNYADSVKG | 203 | KRSWFSQEY | 287 |
| 54G08 | FNLMG | 120 | AITSSSNTNYADSVKG | 204 | QYTITPWGIKKDY | 288 |
| 54G09 | IRDMG | 121 | TITSDQSTNYADSVKG | 205 | RVRTVLRGWRDY | 289 |
| 55B02 | INSMN | 122 | DMRSDGSTNYADSVKG | 206 | NSIFRSRDY | 290 |
| 55B05 | GYTVA | 123 | RISWSGIMAYYAESVKG | 207 | RSQIRSPWSSLDDY | 291 |
| | | | | | DR | |
| 55C05 | MKAMG | 124 | QITRGDSTNYADSVKG | 208 | DRFFGRDY | 292 |
| 55D08 | ISAMG | 126 | TITSAGSSNYSDSVKG | 210 | VYSRPLLGPLEV | 294 |
| 55E07 | IYGMG | 127 | RITSGGSTYYADSVKG | 211 | GVVVATSPKFYAY | 295 |
| 55E09 | TYTMG | 128 | AIIWSGGRTRYADSVKG | 212 | RRLGTGY | 296 |
| 55E10 | IQTIG | 129 | TISSGGSTNYADSVKG | 213 | RYWFRDY | 297 |
| 55F04 | INVRG | 130 | TITSDGSTNYADSVKG | 214 | VRLFRQY | 298 |
| 55F09 | LNAMG | 131 | AITPGGGNTTYADSVKG | 215 | GGSSRWYSSRYYP | 299 |
| | | | | | GGY | |
| 55F10 | RYAMG | 132 | TIRRSGSSTYYLDSTKG | 216 | DSSARALVGGPGN | 300 |
| | | | | | RWDY | |
| 55G02 | INVMG | 133 | FITSGGITNYTDSVKG | 217 | KNAKNVRPGY | 301 |
| 55G08 | INAMR | 134 | SISSGGTTDYVESVKG | 218 | VRFWFPDY | 302 |
| 56A05 | SNTMG | 135 | SISSGGSTNYADSVKG | 219 | RRNVFISS | 303 |
| 56A06 | VYGMG | 136 | RITNIGTTNYADSVKG | 220 | RRLGRDY | 304 |
| 56A09 | LNSMG | 137 | TITRGGTTNYADSVKG | 221 | NFGILVGREY | 305 |
| 56C09 | ILSMA | 138 | NITSVGSTNYADSVKG | 222 | RMPFLGDS | 306 |
| 56C12 | NRAVS | 139 | SISGIRITTYTNSVKG | 223 | NRY | |
| 56D06 | ISAMG | 140 | GISRGGSTKYGDFVKG | 224 | TSITGTYL | 307 |
| 56D07 | MKVMG | 141 | VITSGGRTNYAESVKG | 225 | KTIRPY | 308 |
| 56D10 | ITTMG | 142 | SSSSGGTTNYASSVKG | 226 | RKFITTPWSTDY | 309 |
| 56E04 | HKATG | 143 | KITTGGTTNYADSVKG | 227 | ERYFATTL | 310 |
| 56E05 | NNAGG | 144 | RISSGGNTNYTDSVKG | 228 | QRRVILGPRNY | 311 |
| 56E08 | INDMG | 145 | TITSANITNYADSVKG | 229 | QAKKWRIGPWSDY | 312 |
| 56F07 | INDMA | 146 | IITNDDSTTYADSVKG | 230 | DINTAIWRRKY | 313 |
| 56G07 | IHDMG | 147 | TITPFGRRNYSEYVKG | 231 | RVNGVDY | 314 |
| 56G08 | ISRMG | 148 | TLSRAGTSRYVDSVKG | 232 | AQLGTDY | 315 |
| 56G10 | MYQVG | 149 | EISSRGTTMYADSVKG | 233 | RAFAFGRNS | 316 |
| 56H04 | NKAMG | 150 | RISTVGTAHYADSVKG | 234 | QAGRLYLRNY | 317 |
| 56H05 | FNTMA | 151 | QINNRDNTEYADSVKG | 235 | KRWSWSTGF | 318 |
| 56H07 | LGTMG | 152 | SISTGSTNYADSVKG | 236 | RLWWSNY | 319 |
| 56H08 | VNPMG | 153 | VISSDGSTNYADSVKG | 237 | NRRWSWGSEY | 320 |
| 57A06 | NNAGG | 154 | RISSGGNTNYTDSVKG | 238 | QRRVILGPRNY | 321 |
| 57B01 | INAMA | 155 | RISSGGSTNYADSVKG | 239 | NRHWGWDY | 322 |
| 57B07 | INAMG | 156 | TVDSGGYTNYADSVKG | 240 | GIYKWPWSVDARDY | 323 |
| 57B11 | MNSMG | 157 | LIRSSGGTYYADSVKG | 241 | RRTWLSSES | 324 |
| 57C07 | INTMG | 158 | SISRGGMTNYADSVKG | 242 | GIRSRWYGGPITTY | 325 |
| 57C09 | INAMG | 159 | SISSGGATNYADSVKG | 243 | KKSRWSWSIVHDY | 326 |
| 57D02 | RSDMG | 160 | TITRRSRTNYAEFVKG | 244 | RWGAGGIFST | 327 |
| 57D09 | IDAMG | 161 | SITTGGSTNYADSVKG | 245 | KVRLRWFRPPSDY | 328 |
| 57D10 | ISTMG | 162 | SITKDGTTNYADSVKG | 246 | RATTWVPYRRDAEF | 329 |
| 57E07 | INDMG | 163 | DITRSGSTHYVDSVKG | 247 | DSGSHWWNRRDY | 330 |
| 57E11 | INTMG | 164 | RISRLRVTNYADSVKG | 248 | ANWGLAGNEY | 331 |
| 57G01 | INSMG | 165 | TISNSGTTNYVDAVKG | 249 | QTFWRRNY | 332 |
| 57G07 | INAMG | 166 | TIRRGGNTKYADSVKG | 250 | HSWLDYDY | 333 |
| 57G08 | GITMG | 167 | TIDIHNSTKYADSVKG | 251 | IPTFGRY | 334 |
| 57H08 | TKNVG | 168 | QQRYDGSTNYADSLQG | 252 | NRGFISY | 335 |

Again, such VHHs may be derived in any suitable manner and from any suitable source, and may, for example, be naturally occurring V_{HH} sequences (i.e., from a suitable species of Camelid) or synthetic or semi-synthetic heavy-chain variable domains, including but not limited to “camelized” immunoglobulin sequences (and, in particular, camelized heavy chain variable domain sequences), as well as those that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

This disclosure also encompasses parts, fragments, analogs, mutants, variants, and/or derivatives of the VHHs as disclosed herein and/or polypeptides comprising or essentially consisting of one or more of such parts, fragments, analogs, mutants, variants, and/or derivatives, as long as these parts, fragments, analogs, mutants, variants, and/or derivatives are suitable for the purposes envisaged herein. Such parts, fragments, analogs, mutants, variants, and/or derivatives according to the disclosure are still capable of specifically binding to the sphingolipid target.

### Targets

In certain embodiments, the VHH as taught herein are obtained by affinity selection against a particular pest target. Obtaining suitable polypeptides by affinity selection against a particular pest target may, for example, be performed by screening a set, collection or library of cells that express polypeptides on their surface (e.g., bacteriophages) for binding against a pest target molecule, which molecule is known in the art to be a target for a pesticide; all of which may be performed in a manner known per se, essentially comprising the following non-limiting steps: a) obtaining an isolated solution or suspension of a pest target molecule, which molecule is known to be a target for a pesticide; b) bio-panning phages or other cells from a polypeptide library against the target molecule; c) isolating the phages or other cells binding to the target molecule; d) determining the nucleotide sequence encoding the polypeptide insert from individual binding phages or other cells; e) producing an amount of polypeptide according to this sequence using recombinant protein expression and f) determining the affinity of the polypeptide for the pest target and optionally g) testing the pesticidal activity of the polypeptide in a bioassay for the pest. Various methods may be used to determine the affinity between the polypeptide and the pest target molecule, including, for example, enzyme linked immunosorbent assays (ELISA) or Surface Plasmon Resonance (SPR) assays, which are common practice in the art, for example, as described in Sambrook et al. (2001), Molecular Cloning, A Laboratory Manual, Third Edition, Cold Spring Harbor Laboratory Press, Cold Spring Harbor, N.Y. The dissociation constant is commonly used to describe the affinity between a polypeptide and its pest target molecule. Typically, the dissociation constant of the binding between the polypeptide and its pest target molecule is lower than 10⁻⁵ M, more preferably, the dissociation constant is lower than 10⁻⁶ M, even more preferably, the dissociation constant is lower than 10⁻⁷ M, most preferably, the dissociation constant is lower than 10⁻⁸ M.

Pest target molecules as disclosed herein are molecules occurring in or on pest organisms and that, when bound and/or inhibited, kill or arrest, inhibit or reduce the growth or pesticidal activity of the pest organism. Such suitable target molecules are readily available from existing literature or patent databases for the skilled person and include, without limitation secreted parasitism proteins such as 16D10 as suitable pest target molecules for root knot nematodes (Huang et al (2006) PNAS 103:14302-14306), the V-ATPase proton pump as suitable pest target molecule for coleopteran, hemipteran, dipteran insect species and nematodes (A. J. Knight and C. A. Behm (2011) Ex. Parasitol. September 19), the tetraspanin PLS1 as suitable fungal pest target molecule for B. cinerea and M. grisea (Gourgues et al (2002) Biochem. Biophys. Res. Commun. 297:1197) or the proton-pumping-ATPase as antifungal target (E. K. Manavathu et al. (1999) Antimicrob. Agents and Chemotherapy, December p. 2950). It is understood that preferred pest target molecules are accessible in the extra-cellular space (as opposed to intracellular pest targets).

More particularly, the sphingolipid targets to which the VHHs as disclosed herein bind, constitute a distinctive group of membrane lipids characterized by a long-chain (monounsaturated), di-hydroxy amine structure (sphingosine). Sphingolipids are essential components of the plasma membrane of cells where they are typically found in the outer leaflet. They are membrane constituents of some bacterial groups, particularly anaerobes. These groups include Bacteroides, Prevotella, Porphyromonas, Fusobacterium, Sphingomonas, Sphingobacterium, Bdellovibrio, Cystobacter, Mycoplasma, Flectobacillus, and possibly Acetobacter. Fungi in which sphingolipids have been found comprise Saccharomyces, Candida, Histoplasma, Phytophthora, Cryptococcus, Aspergillus, Neurospora, Schizosaccharomyces, Fusicoccum, Shizophyllum, Amanita, Hansenula, Lactarius, Lentinus, Penicillium, Clitocybe, Paracoccidioides, Agaricus, Sporothrix, and oomycete plant pathogens.

The basic building block of fungal sphingolipids is sphinganine, which can be converted either to ceramide and finally to ceramide monohexosides (CMH; cerebrosides), or to phytoceramide and finally to ceramide dihexosides (CDH) or to glycoinositol phosphorylceramides (GIPCs). Non-limiting examples of sphinglolipids against which the VHH as disclosed herein are directed include for instance 9-methyl 4,8-sphingadienine, glycosylceramides, glucosylceramide, monoglucosylceramides, oligoglucosylceramides, gangliosides, sulfatides, ceramides, sphingosine-1-phosphate, ceramide-1-phosphate, galactosylceramide, inositol-phosphorylceramide (IPC), mannosyl-inositol-phosphorylceramide (MIPC), galactosyl-inositol-phosphorylceramide, mannosyl-(inositol-phosphoryl)₂-ceramide (M(IP)₂C), dimannosyl-inositol-phosphorylceramide (M2IPC), galactosyl-dimannosyl-inositol-phosphorylceramide (GalM2IPC), mannosyl-di-inositol-diphosphorylceramide, di-inositol-diphosphorylceramide, trigalactosyl-glycosylceramide.

Non-limiting examples of sphingolipids against which the VHH as disclosed herein are directed include for instance glycosylceramides, glucosylceramide, sphingomyelin, monoglycosylceramides, oligoglycosylceramides, gangliosides, sulfatides, ceramides, sphingosine-1-phosphate and ceramide-1-phosphate.

In certain preferred embodiments of the transgenic plant or plant tissue or plant cell, methods, or uses, as taught herein, the sphingolipid is a ceramide. In a further preferred embodiment, the sphingolipid is a glycosphingolipid. In a further preferred embodiment, the sphingolipid is a cerebroside (i.e., monoglycosylceramide). In a further preferred embodiment, the sphingolipid is a glucocerebroside (i.e., glucosylceramide).

In certain preferred embodiments of the transgenic plant or plant tissue or plant cell, methods, or uses, as taught herein, the fungal sphingolipid is a fungal ceramide. In a further preferred embodiment, the fungal sphingolipid is a fungal glycosphingolipid. In a further preferred embodiment, the fungal sphingolipid is a fungal cerebroside (i.e., monoglycosylceramide). In a further preferred embodiment, the fungal sphingolipid is a fungal glucocerebroside (i.e., glucosylceramide).

In certain embodiments, the sphingolipid as described herein is glucosylceramide (glucocerebroside) from Pleurotus citrinopileatus.

In certain embodiments of the transgenic plants or plant tissues or plant cells, methods, or uses, as taught herein, the sphingolipid may comprise a C19 sphingoid base with a C-9 methyl group, and two double bonds (Δ4, Δ8).

In certain embodiments of the transgenic plants or plant tissues or plant cells, methods, or uses, as taught herein, the sphingolipid may have, may comprise, consist of, or be represented by any of the following structures:

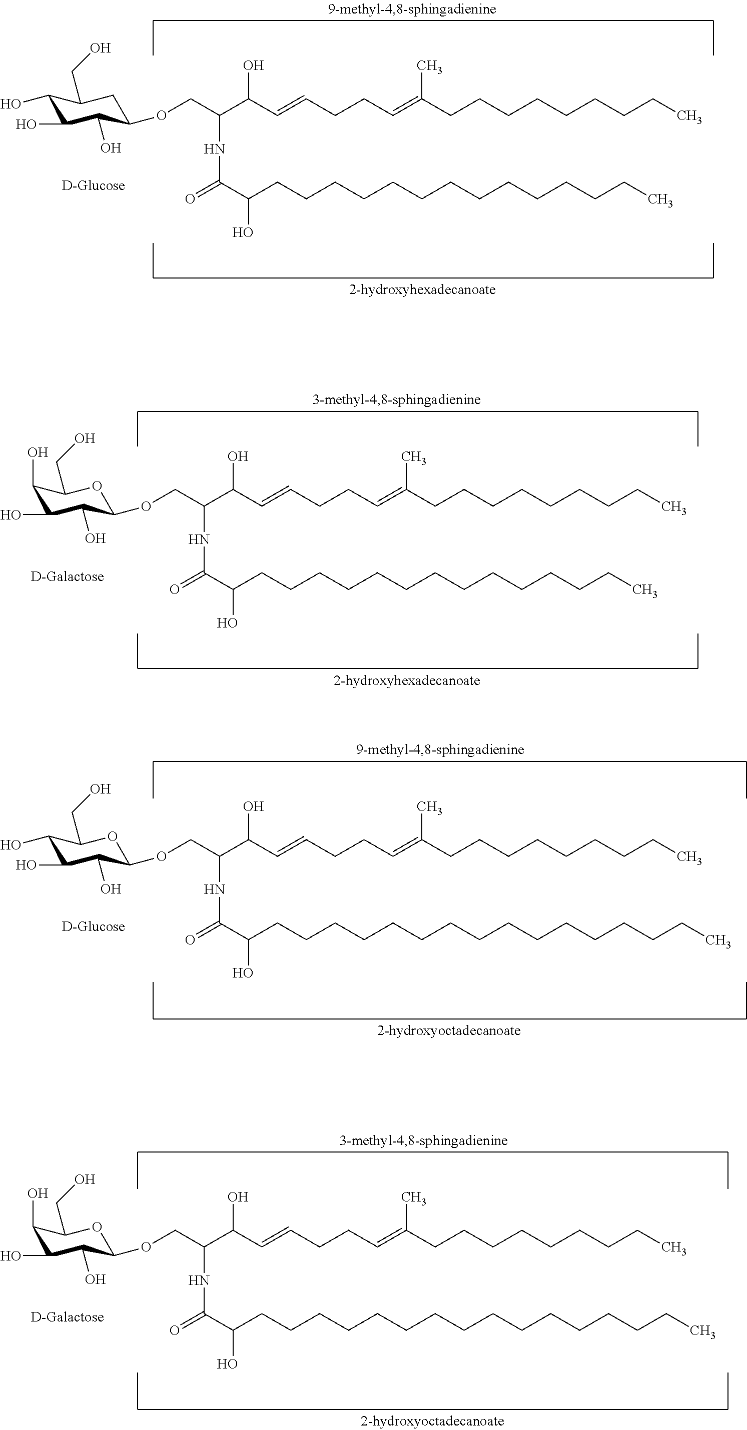

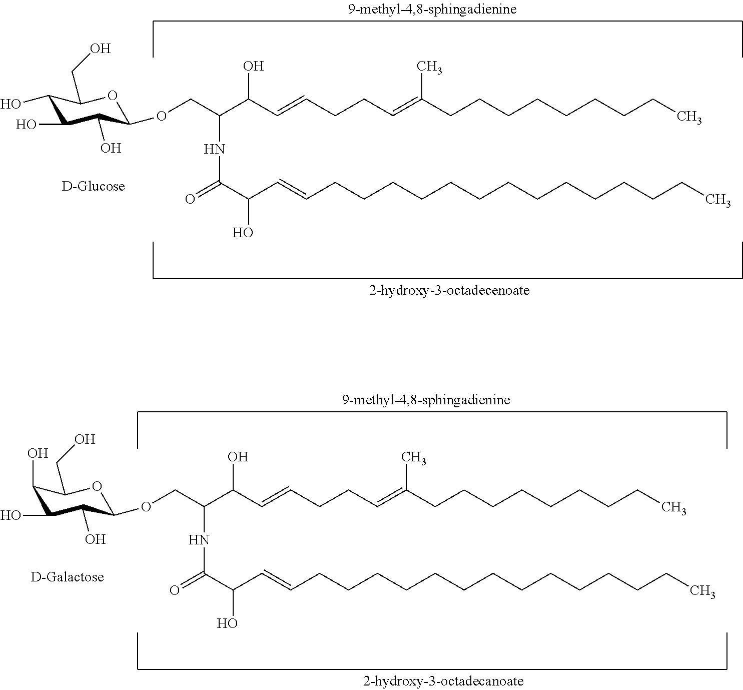

In certain embodiments of the transgenic plants or plant tissues or plant cells, methods, or uses, as taught herein, the sphingolipid as described herein may have, or comprise any of the following structures:

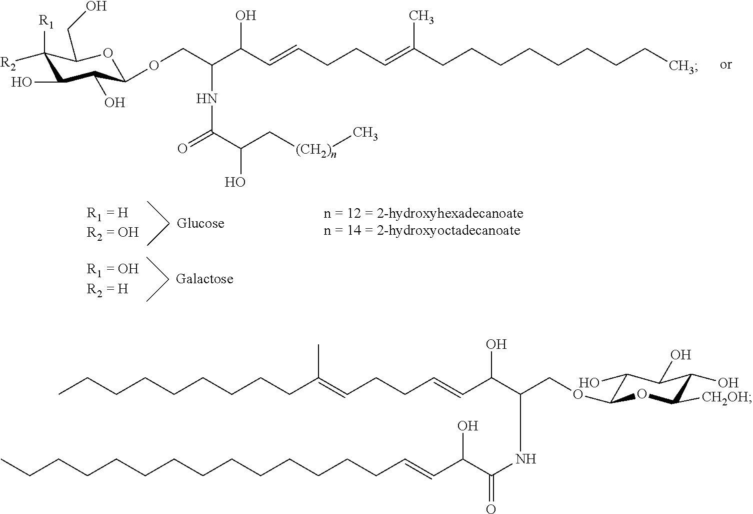

or N-2′-hydroxyhexadecanoyl-1-ß-D-glucopyranosyl-9-methyl-4,8-sphingadienine.

In certain embodiments, the plant pathogen is a fungus, such as a plant pathogenic fungus, as defined before. Fungi can be highly detrimental for plants and can cause substantial harvest losses in crops. Plant pathogenic fungi include necrotrophic fungi and biotrophic fungi, and include ascomycetes, basidiomycetes and oomycetes.

Examples of plant pathogenic fungi are known in the art and include, but are not limited to, those selected from the group consisting of the Genera: Alternaria, Ascochyta, Botrytis, Cercospora, Colletotrichum, Diplodia, Erysiphe, Fusarium, Leptosphaeria, Gaeumanomyces, Helminthosporium, Macrophomina, Nectria, Peronospora, Phoma, Phymatotrichum, Phytophthora, Plasmopara, Podosphaera, Puccinia, Puthium, Pyrenophora, Pyricularia, Pythium, Rhizoctonia, Scerotium, Sclerotinia, Septoria, Thielaviopsis, Uncinula, Venturia; and Verticillium. Specific examples of plant pathogenic fungi that may be combated by the transgenic plants or plant tissues or plant cells, methods, or uses, as taught herein include Erysiphe graminis in cereals, Erysiphe cichoracearum and Sphaerotheca fuliginea in cucurbits, Podosphaera leucotricha in apples, Uncinula necator in vines, Puccinia sp. in cereals, Rhizoctonia sp. in cotton, potatoes, rice and lawns, Ustilago sp. in cereals and sugarcane, Venturia inaequalis (scab) in apples, Helminthosporium sp. in cereals, Septoria nodorum in wheat, Septoria tritici in wheat, Rhynchosporium secalis on barley, Botrytis cinerea (gray mold) in strawberries, tomatoes and grapes, Cercospora arachidicola in groundnuts, Peronospora tabacina in tobacco, or other Peronospora in various crops, Pseudocercosporella herpotrichoides in wheat and barley, Pyrenophera teres in barley, Pyricularia oryzae in rice, Phytophthora infestans in potatoes and tomatoes, Fusarium sp. (such as Fusarium oxysporum) and Verticillium sp. in various plants, Plasmopara viticola in grapes, Alternaria sp. in fruit and vegetables, Pseudoperonospora cubensis in cucumbers, Mycosphaerella fijiensis in banana, Ascochyta sp. in chickpeas, Leptosphaeria sp. on canola, and Colleotrichum sp. in various crops.

In certain embodiments of the transgenic plants or plant tissues or plant cells, methods, or uses, as taught herein, the plant pathogenic fungus that may be combated by the transgenic plants or plant tissues or plant cells include the plant pathogenic fungi as defined in Table 3.

In certain embodiments of the transgenic plants or plant tissues or plant cells, methods, or uses, as taught herein, the plant pathogenic fungus that may be combated by the transgenic plants or plant tissues or plant cells include the plant pathogenic fungi as defined in Table 3 in combination with the transgenic plants or plant tissues or plant cells as defined in Table 3.

TABLE 3
List of plant pathogenic fungi and their combination with transgenic
plants or plant tissues or plant cells, in particular, crops

| | | Common name of fungal |
|---|---|---|
| Crop | Plant pathogenic fungus | disease |
| Banana | Mycosphaerella fi jiensis | Black Sigatoka |
| Banana | Mycosphaerella musicola | Yellow Sigatoka |
| Barley oat rye | Alternaria spp. | Kernel blight |
| Barley oat rye | Puccinia hordei | Leaf rust |
| Barley oat rye | Drechslera graminea = Pyrenophora graminea | Barley stripe |
| Barley oat rye | Pyrenophora teres | Net blotch |
| Barley oat rye | Erysiphe graminis f. sp. hordei | Powdery mildew |
| Barley oat rye | Stagonospora nodorum | Stagonospora blotch |
| Canola | Alternaria spp. | Alternaria blackspot |
| Canola | Leptosphaeria maculans | Blackleg |
| Canola | Sclerotinia sclerotiorum | Sclerotinia stem rot |
| Corn | Puccinia sorghi | Rust |
| Corn | Colletotrichum graminicola | Anthracnose leaf blight |
| Corn | Aureobasidium zeae | Eye spot |
| Corn | Cercospora sorghi | Gray leaf spot |
| Corn | Setosphaeria turcica | Northern corn leaf blight |
| Corn | Cochliobolus carbonum | Northern corn leaf spot |
| Corn | Cochliobolus heterostrophus | Southern corn leaf blight |
| Corn | Rhizoctonia solani | Rhizoctonia root and stalk |
| | | rot |
| Corn | Helmithosporium spp. (maydis, turcicum, | Leaf lights |
| | carbonum) | |
| Corn | Puccinia spp. | Rust |
| Corn | Phytium spp. | |
| Corn | Cercospora zeae-maydis | Gray Leaf Spot |
| Corn | Physoderma maydis | Physoderma Brown Spot |
| Corn | Diplodia maydis | Diplodia Ear Rot |
| Cotton | Glomerella gossypii | Anthracnose |
| Cotton | Ascochyta gossypii | Ascochyta blight, Boll rot |
| Cotton | Fusarium verticillioides | Hardlock |
| Cotton | Puccinia schedonnardi | Cotton rust |
| Cotton | Puccinia cacabata | Southwestern cotton rust |
| Cotton | Glomerella gossypii | Anthracnose |
| Cotton | Puccinia cacabata | Southwestern cotton rust |
| Cotton | Pythium aphanidermatum | Pythium seedling blight |
| Cotton | Rhizoctonia solani | Rhizoctonia seedling blight |
| Potato | Colletotrichum coccodes | Black dot |
| Potato | Alternaria solani | Early blight |
| Potato | Phytophthora infestans | Late blight |
| Potato | Erysiphe cichoracearum | Powdery mildew |
| Potato | Rhizoctonia solani | Black scurf |
| Potato | Helminthosporium solani | Silver scurf |
| Potato | Sclerotinia sclerotiorum | White Mold |
| Rice | Rhizoctonia solani | Sheath blight |
| Rice | Ceratobasidium oryzae-sativae = Rhizoctonia | Aggregate sheath spot |
| | oryzae-sativae | |
| Rice | Gaeumannomyces graminis var. graminis | Black sheath rot |
| Rice | Magnaporthe salvinii = Sclerotium oryzae = | Stem rot |
| | Nakateae sigmoidea | |
| Rice | Cochliobolus miyabeanus | Brown leaf spot |
| Rice | Entyloma oryzae | Leaf smut |
| Rice | Cercospora janseana = Cercospora oryzae | Narrow brown leaf spot |
| Rice | Tilletia barclayana = Neovossia barclayana | Kernel smut |
| Rice | Pyricularia grisea | Leaf/Panicle blast |
| Rice | Ustilaginoidea virens | False Smut |
| Soybean | Rhizoctonia solani | Aerial blight |
| Soybean | Alternaria spp. | Alternaria leaf spot |
| Soybean | Colletotrichum truncatum | Anthracnose |
| Soybean | Septoria glycines | Brown spot |
| Soybean | Cercospora kikuchii | Cercospora blight and leaf |
| | | spot |
| Soybean | Cercospora sojina | Frogeye leaf spot |
| Soybean | Diaporthe phaseolorum | Pod and stem blight |
| Soybean | Phakopsora spp. | Rust |
| Soybean | Rhizoctonia solani | Rhizoctonia solani |
| Soybean | Sclerotium rolfsii | Southern blight |
| Soybean | Sclerotinia sclerotiorum | White Mold |
| Tobacco | Peronospora tabacina | Blue mold |
| Tobacco | Cercospora nicotianae | Frogeye leaf spot |
| Tobacco | Rhizoctonia solani | Target spot |
| Wheat | Puccinia triticina = Puccinia recondita f. sp. | Leaf rust |
| | tritic | |
| Wheat | Septoria tritici, Septoria nodorum | Septoria leaf and glume |
| | | blotch |
| Wheat | Puccinia graminis | Stem rust |
| Wheat | Puccinia striiformis | Stripe rust |
| Wheat | Pyrenophora triticirepentis | Tan spot |
| Wheat | Erysiphe graminis | Powdery mildew |
| Wheat | Blumeria spp., Erysiphe spp. | Powdery mildew |
| Wheat | Stagonospora nordorum | Glume Blotch |
| Wheat | Blumeria spp., Erysiphe spp. | Powdery mildew |
| Wheat | Stagonospora nordorum | Glume Blotch |
| Wheat | Drechslera tritici-repentis | Helminthosporium leaf |
| | | blight |
| Wheat | Bipolaris sorokiniana | Spot Blotch |
| Wheat | Tapesia spp. | Foot Rot/Eyespot |

In certain embodiments of the transgenic plants or plant tissues or plant cells, methods, or uses, as taught herein, the plant pathogenic fungus may be a plant pathogenic fungus from the genus chosen from the group consisting of Alternaria, Ascochyta, Botrytis, Cercospora, Colletotrichum, Diplodia, Erysiphe, Fusarium, Leptosphaeria, Gaeumanomyces, Helminthosporium, Macrophomina, Nectria, Penicillium, Peronospora, Phoma, Phymatotrichum, Phytophthora, Plasmopara, Podosphaera, Puccinia, Pyrenophora, Pyricularia, Pythium, Rhizoctonia, Scerotium, Sclerotinia, Septoria, Thielaviopsis, Uncinula, Venturia, Verticillium, Magnaporthe, Blumeria, Mycosphaerella, Ustilago, Melampsora, Phakospora, Monilinia, Mucor, Rhizopus, and Aspergillus.

In certain embodiments, the transgenic plants or plant tissues or plant cells as taught herein may comprise at least one polynucleotide comprising at least one sequence encoding a VHH, which specifically binds to a sphingolipid of a fungus from the fungal species Botrytis, Fusarium or Penicillium. In further particular embodiments, the fungal sphingolipid is a ceramide, such as, in particular, glucosylceramide.

In particular embodiments, this disclosure provides transgenic plants or plant tissues or plant cells as taught herein may comprise at least one polynucleotide comprising at least one sequence encoding VHHs that are specifically directed against a structural molecular component of the fungus, i.e., a fungal sphingolipid. The applicants have surprisingly succeeded in identifying such VHHs while it is generally described in the art that it is (technically) difficult to generate proteins or amino acid sequences having a unique and specific interaction with non-protein molecular structures.

Based on the present teaching, further non-limiting examples of suitable fungal pest target molecules can be envisaged by the person skilled in the art and comprise, for example, chitin synthase, β-1,3-glucan synthase, succinate dehydrogenase, fungal glycosylceramides, or the tetraspanin PLS1.

Also disclosed herein are plant pathogenic bacteria including, but not limited to, Acidovorax avenae subsp. avenae (causing bacterial brown stripe of rice), Acidovorax avenae subsp. cattleyae (causing bacterial brown spot of cattleya), Acidovorax konjaci Konnyaku (causing bacterial leaf blight), Agrobacterium rhizogenes (causing hairy root of melon), Agrobacterium tumefaciens (causing crown gall), Burkholderia andropogonis (causing bacterial spot of carnation), Burkholderia caryophylli (causing bacterial wilt of carnation), Burkholderia cepacia (causing bacterial brown spot of cymbidium), Burkholderia gladioli pv. gladioli (causing neck rot of gladiolus), Burkholderia glumae (causing bacterial grain rot of rice), Burkholderia plantarii (causing bacterial seedling blight of rice), Clavibacter michiganensis subsp. michiganensis (causing bacterial canker of tomato), Clavibacter michiganensis subsp. sepedonicus (causing ring rot of potato), Clostridium spp. (causing slimy rot of potato), Curtobacterium flaccumfaciens (causing bacterial canker of onion), Erwinia amylovora (causing fire blight of pear), Erwinia ananas (causing bacterial palea browning of rice), Erwinia carotovora subsp. atroseptica (causing black leg of potato), Erwinia carotovora subsp. carotovora (causing bacterial soft rot of vegetables), Erwinia chrysanthemi (causing bacterial seedling blight of taro), Erwinia chrysanthemi pv. zeae (causing bacterial foot rot of rice), Erwinia herbicola pv. millettiae (causing bacterial gall of wisteria), Pseudomonas cichorii (causing bacterial spot of chrysanthemum), Pseudomonas corrugate Pith (causing necrosis of tomato), Pseudomonas fuscovaginae (causing sheath brown rot of rice), Pseudomonas marginalis pv. marginalis (causing soft rot of cabbage) Pseudomonas rubrisubalbicans (causing mottled stripe of sugar cane), Pseudomonas syringae pv. aptata (causing bacterial blight of sugar beet), Pseudomonas syringae pv. atropurpurea (causing halo blight of ryegrass), Pseudomonas syringae pv. castaneae (causing bacterial canker of chestnut), Pseudomonas syringae pv. glycinea (causing bacterial blight of soybean), Pseudomonas syringae pv. lachrymans (causing bacterial spot of cucumber), Pseudomonas syringae pv. maculicola (causing bacterial black spot of cabbage), Pseudomonas syringae pv. mori (causing bacterial blight of mulberry), Pseudomonas syringae pv. morsprunorum (causing bacterial canker of plums), Pseudomonas syringae pv. oryzae (causing halo blight of rice), Pseudomonas syringae pv. phaseolicola (causing halo blight of kidney bean), Pseudomonas syringae pv. pisi (causing bacterial blight of garden pea), Pseudomonas syringae pv. sesame (causing bacterial spot of sesame), Pseudomonas syringae pv. striafaciens (causing bacterial stripe blight of oats), Pseudomonas syringae pv. syringae (causing bacterial brown spot of small red bead), Pseudomonas syringae pv. tabaci (causing wild fire of tobacco), Pseudomonas syringae pv. theae (causing bacterial shoot blight of tea), Pseudomonas syringae pv. tomato (causing bacterial leaf spot of tomato), Pseudomonas viridiflava (causing bacterial brown spot of kidney bean), Ralstonia solanacearum (causing bacterial wilt), Rathayibacter rathayi (causing bacterial head blight of orchardgrass), Streptomyces scabies (causing common scab of potato), Streptomyces ipomoea (causing soil rot of sweet potato), Xanthomonas albilineans (causing white streak of sugar cane), Xanthomonas campestris pv. cerealis (causing bacterial streak of rye), Xanthomonas campestris pv. campestris (causing black rot), Xanthomonas campestris pv. citri (causing canker of citrus), Xanthomonas campestris pv. cucurbitae (causing bacterial brown spot of cucumber), Xanthomonas campestris pv. glycines (causing bacterial pastule of soybean), Xanthomonas campestris pv. incanae (causing black rot of stock), Xanthomonas campestris pv. (causing angular leaf spot of cotton malvacearum), Xanthomonas campestris pv. (causing bacterial canker of mango), Mangiferaeindicae Xanthomonas campestris pv. mellea (causing wisconsin bacterial leaf spot of tobacco), Xanthomonas campestris pv. (causing bacterial spot of great nigromaculans burdock), Xanthomonas campestris pv. phaseoli (causing bacterial pastule of kidney bean), Xanthomonas campestris pv. pisi (causing bacterial stem-rot of kidney bean), Xanthomonas campestris pv. pruni (causing bacterial shot hole of peach), Xanthomonas campestris pv. raphani (causing bacterial spot of Japanese radish), Xanthomonas campestris pv. ricini (causing bacterial spot of castor-oil plant), Xanthomonas campestris pv. theicola (causing canker of tea), Xanthomonas campestris pv. translucens (causing bacterial blight of orchardgrass), Xanthomonas campestris pv. vesicatoria (causing bacterial spot of tomato), Xanthomonas oryzae pv. oryzae (causing bacterial leaf blight of rice).

Also disclosed herein are plant pests such as insects, arachnids, helminths, viruses, nematodes and molluscs encountered in agriculture, in horticulture, in forests, in gardens and in leisure facilities. The transgenic plants or plant tissues or plant cells as taught herein are active against normally sensitive and resistant species and against all or some stages of development. These plant pests include: pests from the phylum: Arthropoda, in particular, from the class of the arachnids, for example, Acarus spp., Aceria sheldoni, Aculops spp., Aculus spp., Amblyomma spp., Amphitetranychus viennensis, Argas spp., Boophilus spp., Brevipalpus spp., Bryobia praetiosa, Centruroides spp., Chorioptes spp., Dermanyssus gallinae, Dermatophagoides pteronyssius, Dermatophagoides farinae, Dermacentor spp., Eotetranychus spp., Epitrimerus pyri, Eutetranychus spp., Eriophyes spp., Halotydeus destructor, Hemitarsonemus spp., Hyalomma spp., Ixodes spp., Latrodectus spp., Loxosceles spp., Metatetranychus spp., Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vaejovis spp., and Vasates lycopersici.

Still other examples are from the order of the Anoplura (Phthiraptera), for example, Damalinia spp., Haematopinus spp., Linognathus spp., Pediculus spp., Ptirus pubis, and Trichodectes spp.

Still other examples are from the order of the Chilopoda, for example, Geophilus spp., and Scutigera spp.

Still other examples are from the order of the Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptorhynchus lapathi, Cylindrocopturus spp., Dermestes spp., Diabrotica spp., Dichocrocis spp., Diloboderus spp., Epilachna spp., Epitrix spp., Faustinus spp., Gibbium psylloides, Hellula undalis, Heteronychus arator, Heteronyx spp., Hylamorpha elegans, Hylotrupes bajulus, Hypera postica, Hypothenemus spp., Lachnosterna consanguinea, Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllotreta spp., Popillia japonica, Premnotrypes spp., Prostephanus truncatus, Psylliodes spp., Ptinus spp., Rhizobius ventralis, Rhizopertha dominica, Sitophilus spp., Sphenophorus spp., Stegobium paniceum, Sternechus spp., Symphyletes spp., Tanymecus spp., Tenebrio molitor, Tribolium spp., Trogoderma spp., Tychius spp., Xylotrechus spp., and Zabrus spp.

Still other examples are from the order of the Collembola, for example, Onychiurus armatus.

Still other examples are from the order of the Diplopoda, for example, Blaniulus guttulatus.

Still other examples are from the order of the Diptera, for example, Aedes spp., Agromyza spp., Anastrepha spp., Anopheles spp., Asphondylia spp., Bactrocera spp., Bibio hortulanus, Calliphora erythrocephala, Ceratitis capitata, Chironomus spp., Chrysomyia spp., Chrysops spp., Cochliomyia spp., Contarinia spp., Cordylobia anthropophaga, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hylemyia spp., Hyppobosca spp., Hypoderma spp., Liriomyza spp., Lucilia spp., Lutzomia spp., Mansonia spp., Musca spp., Nezara spp., Oestrus spp., Oscinella frit, Pegomyia spp., Phlebotomus spp., Phorbia spp., Phormia spp., Prodiplosis spp., Psila rosae, Rhagoletis spp., Sarcophaga spp., Simulium spp., Stomoxys spp., Tabanus spp., Tannia spp., Tetanops spp., Tipula spp.

Still other examples are from the order of the Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., and Triatoma spp.

Still other examples are from the order of the Homoptera, for example, Acyrthosipon spp., Acrogonia spp., Aeneolamia spp., Agonoscena spp., Aleurodes spp., Aleurolobus barodensis, Aleurothrixus spp., Amrasca spp., Anuraphis cardui, Aonidiella spp., Aphanostigma pin, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Ferrisia spp., Geococcus coffeae, Hieroglyphus spp., Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes spp., Trioza spp., Typhlocyba spp., Unaspis spp., Viteus vitifolii, and Zygina spp.

Still other examples are from the order of the Hymenoptera, for example, Acromyrmex spp., Athalia spp., Atta spp., Diprion spp., Hoplocampa spp., Lasius spp., Monomorium pharaonis, Solenopsis invicta, Tapinoma spp., and Vespa spp.

Still other examples are from the order of the Isopoda, for example, Armadillidium vulgare, Oniscus asellus, and Porcellio scaber.

Still other examples are from the order of the Isoptera, for example, Coptotermes spp., Cornitermes cumulans, Cryptotermes spp., Incisitermes spp., Microtermes obesi, Odontotermes spp., and Reticulitermes spp.

Still other examples are from the order of the Lepidoptera, for example, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Chematobia brumata, Chilo spp., Choristoneura spp., Clysia ambiguella, Cnaphalocerus spp., Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Mocis spp., Mythimna separata, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudoplusia includens, Pyrausta nubilalis, Rachiplusia nu, Schoenobius spp., Scirpophaga spp., Scotia segetum, Sesamia spp., Sparganothis spp., Spodoptera spp., Stathmopoda spp., Stomopteryx subsecivella, Synanthedon spp., Tecia solanivora, Thermesia gemmatalis, Tinea pellionella, Tineola bisselliella, Tortrix spp., Trichophaga tapetzella, Trichoplusia spp., Tuta absoluta, Virachola spp.

Still other examples are from the order of the Orthoptera, for example, Acheta domesticus, Blatta orientalis, Blattella germanica, Dichroplus spp., Gryllotalpa spp., Leucophaea maderae, Locusta spp., Melanoplus spp., Periplaneta spp., Pulex irritans, Schistocerca gregaria, and Supella longipalpa.

Still other examples are from the order of the Siphonaptera, for example, Ceratophyllus spp., Ctenocephalides spp., Tunga penetrans, and Xenopsylla cheopis.

Still other examples are from the order of the Symphyla, for example, Scutigerella spp.

Still other examples are from the order of the Thysanoptera, for example, Anaphothrips obscurus, Baliothrips biformis, Drepanothris reuteri, Enneothrips flavens, Frankliniella spp., Heliothrips spp., Hercinothrips femoralis, Rhipiphorothrips cruentatus, Scirtothrips spp., Taeniothrips cardamoni, and Thrips spp.

Still other examples are from the order of the Zygentoma (=Thysanura), for example, Lepisma saccharina, Thermobia domestica, for example, Lepisma saccharina, and Thermobia domestica.

In another embodiment, pests of the phylum Mollusca, in particular, from the class of the Bivalvia, for example, Dreissena spp., are also important plant pests.

In another embodiment, pests of the class of the Gastropoda are important plant pests, for example, Anion spp., Biomphalaria spp., Bulinus spp., Deroceras spp., Galba spp., Lymnaea spp., Oncomelania spp., Pomacea spp., and Succinea spp.

In yet another embodiment, plant pests are from the phylum Nematoda are important plant pests, i.e., phytoparasitic nematodes, thus meaning plant parasitic nematodes that cause damage to plants. Plant nematodes encompass plant parasitic nematodes and nematodes living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonerna spp.; sedentary parasites such as Heterodera spp., Globodera spp., and Meloidogyne spp., and stem and leaf endoparasites such as Ditylenchus spp., Aphelenchoides spp., and Hirshmaniella spp. In addition, harmful root parasitic soil nematodes are cyst-forming nematodes of the genera Heterodera or Globodera, and/or root knot nematodes of the genus Meloidogyne. Harmful species of these genera are, for example, Meloidogyne incognata, Heterodera glycines (soybean cyst nematode), Globodera pallida and Globodera rostochiensis (potato cyst nematode). Still other important genera of importance as plant pests comprise Rotylenchulus spp., Paratriclodorus spp., Pratylenchus penetrans, Radolophus simuli, Ditylenchus dispaci, Tylenchulus semipenetrans, Xiphinema spp., and Bursaphelenchus spp., and the like, in particular, Aphelenchoides spp., Bursaphelenchus spp., Ditylenchus spp., Globodera spp., Heterodera spp., Longidorus spp., Meloidogyne spp., Pratylenchus spp., Radopholus similis, Trichodorus spp., Tylenchulus semipenetrans, and Xiphinema spp.

Also disclosed herein as being plant pests are plant viruses selected from analfamovirus, an allexivirus, an alphacryptovirus, an anulavirus, an apscaviroid, an aureusvirus, an avenavirus, an aysunviroid, a badnavirus, a begomovirus, a benyvirus, a betacryptovirus, a betaflexiviridae, a bromovirus, a bymovirus, a capillovirus, a carlavirus, a carmovirus, a caulimovirus, a cavemovirus, a cheravirus, a closterovirus, a cocadviroid, a coleviroid, a comovirus, a crinivirus, a cucumovirus, a curtovirus, a cytorhabdovirus, a dianthovirus, an enamovirus, an umbravirus and B-type satellite virus, a fabavirus, a fijivirus, a furovirus, a hordeivirus, a hostuviroid, an idaeovirus, an ilarvirus, an ipomovirus, a luteovirus, a machlomovirus, a macluravirus, a marafivirus, a mastrevirus, a nanovirus, a necrovirus, a nepovirus, a nucleorhabdovirus, an oleavirus, an ophiovirus, an oryzavirus, a panicovirus, a pecluvirus, a petuvirus, a phytoreovirus, a polerovirus, a pomovirus, a pospiviroid, a potexvirus, a potyvirus, a reovirus, a rhabdovirus, a rymovirus, a sadwavirus, a SbCMV-like virus, a sequivirus, a sobemovirus, a tenuivirus, a TNsatV-like satellite virus, a tobamovirus, a topocuvirus, a tospovirus, a trichovirus, a tritimovirus, a tungrovirus, a tymovirus, an umbravirus, a varicosavirus, a vitivirus, or a waikavirus.

In certain embodiments, this disclosure provides a transgenic plant or plant tissue or plant cell resistant to a plant pathogenic fungus, wherein the transgenic plant or plant tissue or plant cell comprises at least one polynucleotide comprising at least one sequence encoding a VHH specifically binding to a sphingolipid of a fungus.

In certain embodiments, this disclosure provides a transgenic plant or plant tissue or plant cell resistant to an infection or other biological inter-reaction with a plant pathogenic fungus, wherein the transgenic plant or plant tissue or plant cell comprises at least one polynucleotide comprising at least one sequence encoding a VHH specifically binding to a sphingolipid of a fungus.

### Methods and Uses

A further aspect provides a method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, comprising expressing in at least part of the plant or plant tissue or plant cell at least one polynucleotide encoding a VHH specifically binding to a pathogen.

In certain embodiments, this disclosure provides methods for protecting or treating a plant or a part of a plant from an infection or other biological interaction with a plant pathogen, at least comprising the step of expressing in the plant or plant tissue or plant cell or in at least part of the plant or plant tissue or plant cell (i.e., in planta) a polynucleotide comprising at least one sequence encoding a VHH specifically binding to a pathogen, in particular, to a sphingolipid of a fungus. In certain embodiments, the polynucleotide may be expressed in the plant or plant tissue or plant cell or in at least part of the plant or plant tissue or plant cell (i.e., in planta) under conditions effective to protect or treat at least part of the plant or plant tissue or plant cell against that infection or biological interaction with the plant pathogen, in particular, with a plant pathogenic fungus.

In certain particular embodiments, this disclosure provides methods of inhibiting, preventing, reducing or controlling the growth of a plant pathogen, in particular, a plant pathogenic fungus, comprising at least the step of expressing in at least part of the plant or plant tissue or plant cell at least one polynucleotide encoding a VHH specifically binding to a pathogen, in particular, to a sphingolipid of a fungus.

In certain other embodiments, this disclosure provides methods for killing a plant pathogen, in particular, a plant pathogenic fungus, comprising at least the step of expressing in at least part of the plant or plant tissue or plant cell at least one polynucleotide encoding a VHH specifically binding to a pathogen, in particular, to a sphingolipid of a fungus.

In certain embodiments, this disclosure provides the use of at least one variable domain of a heavy-chain antibody (VHH) specifically binding to a pathogen, as a antimicrobial agent, preferably as a microbiostatic agent, wherein the VHH is encoded by a polynucleotide that is expressed in at least part of the plant or plant tissue.

In certain embodiments, this disclosure provides the use of the polynucleotides of SEQ ID NO:336 and/or SEQ ID NO:337 for protecting a transgenic plant or plant tissue or plant cell against a plant pathogen, preferably a plant pathogenic fungus.

In certain embodiments, this disclosure provides the use of the polynucleotides of SEQ ID NO:336 and/or SEQ ID NO:337 for improving the yield of a transgenic plant or plant tissue.

The disclosure further provides methods for preparing or generating the VHH as taught herein, as well as methods for producing polynucleotides encoding these. Some preferred but non-limiting examples of such methods will become clear from the further description herein.

As will be clear to the skilled person, one particularly useful method for preparing VHH sequences as disclosed herein generally comprises the steps of:
a) expressing a nucleotide sequence encoding a heavy chain variable domain sequence as disclosed herein or a vector or genetic construct a nucleotide sequence encoding that heavy chain variable domain sequence; and
b) optionally isolating and/or purifying the VHH sequence.

In particular embodiments envisaged herein, the pest-specific a VHH sequences can be obtained by methods that involve generating a random library of amino acid sequences and screening this library for an amino acid sequence capable of specifically binding to a sphingolipid target.

Accordingly, in particular embodiments, methods for preparing a heavy chain variable domain sequence as disclosed herein comprise the steps of
a) providing a set, collection or library of amino acid sequences of a heavy chain variable domain sequences;
b) screening the set, collection or library of amino acid sequences for amino acid sequences that can bind to and/or have affinity for the sphingolipid target; and
c) isolating the amino acid sequence(s) that can bind to and/or have affinity for the sphingolipid target.

In such a method, the set, collection or library of amino acid sequences may be any suitable set, collection or library of amino acid sequences. For example, the set, collection or library of amino acid sequences may be a set, collection or library of immunoglobulin fragment sequences (as described herein), such as a naïve set, collection or library of immunoglobulin fragment sequences; a synthetic or semi-synthetic set, collection or library of immunoglobulin fragment sequences; and/or a set, collection or library of immunoglobulin fragment sequences that have been subjected to affinity maturation.

In particular embodiments of this method, the set, collection or library of amino acid sequences may be an immune set, collection or library of immunoglobulin fragment sequences, for example, derived from a mammal that has been suitably immunized with a sphingolipid target or with a suitable antigenic determinant based thereon or derived therefrom, such as an antigenic part, fragment, region, domain, loop or other epitope thereof. In one particular aspect, the antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example, on the basis of the further disclosure herein. Reference is also made to the review by Hoogenboom in Nature Biotechnology 23, 9:1105-1116 (2005).

In other embodiments, the methods for generating the VHH sequences as disclosed herein comprise at least the steps of:
a) providing a collection or sample of cells expressing heavy chain variable domain amino acid sequences;
b) screening the collection or sample of cells for cells that express an amino acid sequence that can bind to and/or have affinity for a sphingolipid target; and
c) either (i) isolating the amino acid sequence; or (ii) isolating from the cell a nucleic acid sequence that encodes the amino acid sequence, followed by expressing the amino acid sequence.

The collection or sample of cells may, for example, be a collection or sample of B-cells. Also, in this method, the sample of cells may be derived from a mammal that has been suitably immunized with a fungal target or with a suitable antigenic determinant based thereon or derived therefrom, such as an antigenic part, fragment, region, domain, loop or other epitope thereof. In one particular embodiment, the antigenic determinant may be an extracellular part, region, domain, loop or other extracellular epitope(s).

In other embodiments, the method for generating a heavy chain variable domain sequence directed against a sphingolipid target may comprise at least the steps of:
a) providing a set, collection or library of nucleic acid sequences encoding a heavy chain variable domain amino acid sequence;
b) screening the set, collection or library of nucleic acid sequences for nucleic acid sequences that encode an amino acid sequence that can bind to and/or has affinity for the sphingolipid target; and
c) isolating the nucleic acid sequence, followed by expressing the amino acid sequence.

In the above methods, the set, collection or library of nucleic acid sequences encoding amino acid sequences may, for example, be a set, collection or library of nucleic acid sequences encoding a naïve set, collection or library of immunoglobulin fragment sequences; a set, collection or library of nucleic acid sequences encoding a synthetic or semi-synthetic set, collection or library of immunoglobulin fragment sequences; and/or a set, collection or library of nucleic acid sequences encoding a set, collection or library of immunoglobulin fragment sequences that have been subjected to affinity maturation.

In particular, in such a method, the set, collection or library of nucleic acid sequences encodes a set, collection, or library of VHHs. For example, the set, collection or library of nucleic acid sequences may encode a set, collection or library of domain antibodies or single domain antibodies, or a set, collection or library of amino acid sequences that are capable of functioning as a domain antibody or single domain antibody. In specific embodiments, the set, collection or library of nucleotide sequences encodes a set, collection or library of VHH sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example, on the basis of the further disclosure herein. Reference is also made to the review by Hoogenboom in Nature Biotechnology 23, 9:1105-1116 (2005).

The disclosure also relates to amino acid sequences that are obtainable or obtained by the above methods, or alternatively by a method that comprises one of the above methods and in addition at least the steps of determining the nucleotide sequence or amino acid sequence of the immunoglobulin sequence; and of expressing or synthesizing the amino acid sequence in a manner known per se, such as by expression in a suitable host cell or host organism or by chemical synthesis.

In some cases, the methods for producing the amino acid sequences binding specifically to a fungal target as envisaged herein may further comprise the step of isolating from the amino acid sequence library at least one heavy chain variable domain having detectable binding affinity for, or detectable in vitro effect on a sphingolipid target.

These methods may further comprise the step of amplifying a sequence encoding at least one heavy chain variable domain having detectable binding affinity for, or detectable in vitro effect on the activity of a sphingolipid target. For example, a phage clone displaying a particular amino acid sequence, obtained from a selection step of a method described herein, may be amplified by reinfection of a host bacteria and incubation in a growth medium.

In particular embodiments, these methods may encompass determining the sequence of the one or more amino acid sequences capable of binding to a sphingolipid target.

Where a heavy chain variable domain sequence, comprised in a set, collection or library of amino acid sequences, is displayed on a suitable cell or phage or particle, it is possible to isolate from the cell or phage or particle, the nucleotide sequence that encodes that amino acid sequence. In this way, the nucleotide sequence of the selected amino acid sequence library member(s) can be determined by a routine sequencing method.

In further particular embodiments, the methods for producing a heavy chain variable domain as envisaged herein comprise the step of expressing the nucleotide sequence(s) in a host organism under suitable conditions, so as to obtain the actual desired amino acid sequence. This step can be performed by methods known to the person skilled in the art.

In addition, the obtained heavy chain variable domain sequences having detectable binding affinity for, or detectable in vitro effect on the activity of a sphingolipid target, may be synthesized as soluble protein construct, optionally after their sequence has been identified.

For instance, the heavy chain variable domain sequences obtained, obtainable or selected by the above methods can be synthesized using recombinant or chemical synthesis methods known in the art. Also, the amino acid sequences obtained, obtainable or selected by the above methods can be produced by genetic engineering techniques. Thus, methods for synthesizing the heavy chain variable domain sequences obtained, obtainable or selected by the above methods may comprise transforming or infecting a host cell with a nucleic acid or a vector encoding an amino acid sequence having detectable binding affinity for, or detectable in vitro effect on the activity of a sphingolipid target. Accordingly, the amino acid sequences having detectable binding affinity for, or detectable in vitro effect on the activity of a sphingolipid target can be made by recombinant DNA methods. DNA encoding the amino acid sequences can be readily synthesized using conventional procedures. Once prepared, the DNA can be introduced into expression vectors, which can then be transformed or transfected into host cells such as E. coli or any suitable expression system, in order to obtain the expression of amino acid sequences in the recombinant host cells and/or in the medium in which these recombinant host cells reside.

It should be understood, as known by someone skilled in the art of protein expression and purification, that the VHH as taught herein produced from an expression vector using a suitable expression system may be tagged (typically at the N-terminal or C-terminal end of the amino acid sequence) with, e.g., a His-tag or other sequence tag for easy purification.

Transformation or transfection of nucleic acids or vectors into host cells may be accomplished by a variety of means known to the person skilled in the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Suitable host cells for the expression of the desired heavy chain variable domain sequences may be any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic plant.

Thus, the application also provides methods for the production of heavy chain variable domain sequences having detectable binding affinity for, or detectable in vitro effect on the activity of a sphingolipid target comprising transforming, transfecting or infecting a host cell with nucleic acid sequences or vectors encoding such amino acid sequences and expressing the amino acid sequences under suitable conditions.

In other particular embodiments of these methods, the step of obtaining at least one heavy chain variable domain or functional fragment thereof, which specifically binds to a sphingolipid of a plant pathogen comprises:
a) providing a set, collection or library of VHHs or functional variants thereof,
b) screening the set, collection or library of VHHs or functional variants thereof for sequences that specifically bind to and/or have affinity for a sphingolipid of a plant pathogen, and optionally
c) isolating the VHHs or functional variants thereof that specifically bind to and/or have affinity for a sphingolipid of a plant pathogen.

The following non-limiting Examples describe methods and means according to the disclosure. Unless stated otherwise in the Examples, all techniques are carried out according to protocols standard in the art. The following examples are included to illustrate embodiments of the disclosure. Those of skill in the art should, in light of the present disclosure, appreciate that many changes can be made in the specific embodiments that are disclosed and still obtain a like or similar result without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Thus, the Figures, Sequence Listing and the Experimental Part/Examples are only given to further illustrate the disclosure and should not be interpreted or construed as limiting the scope of the disclosure and/or of the appended claims in any way, unless explicitly indicated otherwise herein.

The above disclosure will now be further described by means of the following non-limiting Examples.

### EXAMPLES

### Example 1

Isolation of Nucleic Acid Sequences Encoding Peptides with Affinity for Fungal Glucosylceramide

Animal Immunizations:

VHHs were generated from llamas immunized with fungal glucosylceramide (GlcCer). Llamas were immunized according to standard protocols with 6 boosts of thin Layer Chromatography (TLC)-purified (99%) glucosylceramide (GlcCer) from Pleurotus citrinopileatus (Nacalai Tesque). Purified GlcCer was dissolved in a water:methanol:chloroform mixture and spotted on a TLC silica glass plate. Silica with adsorbed GlcCer was scraped from the plate and suspended in phosphate buffer. The suspension was sonicated, mixed with Freund incomplete adjuvant, and used for subcutaneous injections. VHH were also generated from llamas immunized with native germinated fungal or oomycete spores. Llamas were immunized according to standard protocols with 6 boosts of native germinated spores of Botrytis cinerea or Phytophthora infestans by subcutaneous injections. All llamas remained healthy throughout the immunization process and blood samples were taken before and after immunizations.

Library Construction:

A phage library of antibodies is a phage population in which each individual phage exposes a unique antigen-binding antibody domain on its surface as a part of a chimeric pIII protein. Peripheral blood mononuclear cells were prepared from blood samples of the immunized llamas using Ficoll-Hypaque according to the manufacturer's instructions. Total RNA was extracted from these cells and used as starting material for RT-PCR to amplify VHH encoding gene fragments. These fragments were cloned into phagemid vector pASF20. pASF20 is an expression vector that is derived from pUC119, which contains the lacZ promotor, a synthetic leader sequence, a multiple cloning site, a coliphage pIII protein coding sequence, a resistance gene for ampicillin, and an M13 phage origin for single strand production. In frame with the VHH coding sequence, the vector codes for a C-terminal (His)6 peptide tag and c-myc peptide tag. Phages were prepared according to standard methods (Phage Display of Peptides and Proteins: A Laboratory Manual; Brian K. Kay, Jill Winter, Dr. John McCafferty). Four libraries, each with a clonal diversity equal to or greater than 1E+08, were obtained and phage were produced ensuring presentation of the antibody diversity.

VHH Selections by Phage Display:

Phage expressing antigen-binding antibody domains specific for a particular antigen were isolated by selecting the phage in the library for binding to the antigen. Fungal GlcCer were immobilized on polystyrene Maxisorp multiwell plates by dissolving fungal GlcCer in a water:methanol:chloroform mixture or methanol at different concentrations, adding dissolved fungal GlcCer to wells of the multiwell plate, and allowing to dry overnight at room temperature. Wells with coated fungal GlcCer were washed and blocked with 1% fish gelatin in preparation of VHH selections by phage display. VHH library phages were allowed to bind for two hours at room temperature to wells of 96-well plate coated with fungal GlcCer. To specifically select for phage binding to fungal GlcCer phage were pre-incubated with 1% fish gelatin and/or BSA and/or skimmed milk and/or plant GlcCer and/or mammalian GlcCer. Non-bound phage were removed by extensive washing and bound phage were eluted by competitive elution with RsAFP2 (Osborn et al., 1995) or with trypsin. One to three consecutive rounds of selection were performed, and the titers of phage from fungal GlcCer-coated wells were compared to titers of phage from blank wells and non-target pathogen sphingolipids for enrichment and specificity, respectively. Enrichments were observed in first and subsequent rounds of selection, and phage populations after one or more selection rounds already showed specificity for fungal GlcCer in ELISA (not shown). Individual clones were picked from first, second and/or third round selections for further characterization by sequence analysis and primary binding assays.

VHH Characterization by Sequencing and Binding Assays:

The diversity of the obtained antibody or antibody domain population can be rapidly determined using high-throughput DNA sequencing and allows precise quantification of clonal diversity. Antibody or antibody domain binding and specificity of binding to an antigen can be analyzed in assays for binding to that antigen and compared to related and unrelated controls. Each antibody or antibody domain can bind to a specific antigen and possibly to antigenic variants of that antigen. Specificity is the degree to which the binding of an antibody or antibody domain discriminates between antigenic variants. From individual VHH clones that were picked from first, second or third round phage display selections the DNA was amplified in a colony PCR and PCR products were sequenced by Sanger-sequencing. After sequence analysis and based on sequence diversity, VHH were selected for further characterization. To check for species specificity, fungal and non-fungal GlcCer from target and non-target species were used in binding assays. Primary binding assays to identify which clones were functionally selected from the libraries were performed with TLC-purified (99%) GlcCer or GlcCer-enriched Glycosphingolipids (GSL) fractions from A. brassicicola, B. cinerea, C. beticola, F. culmorum, F. graminearum, F. oxysporum, P. citrinopileatus P. digitatum, P. expansum, or V. dahlia (prepared as described in Ternes et al., 2011 JBC 286:11401-14). GlcCer from soybean and porcine GlcCer were purchased from Avanti Polar Lipids. VHH were produced in 96-well deep-well plates and the binding profile of diluted crude VHH-containing periplasmic extracts was assessed in ELISA format. In the same way, binding assays were performed with purified VHH.

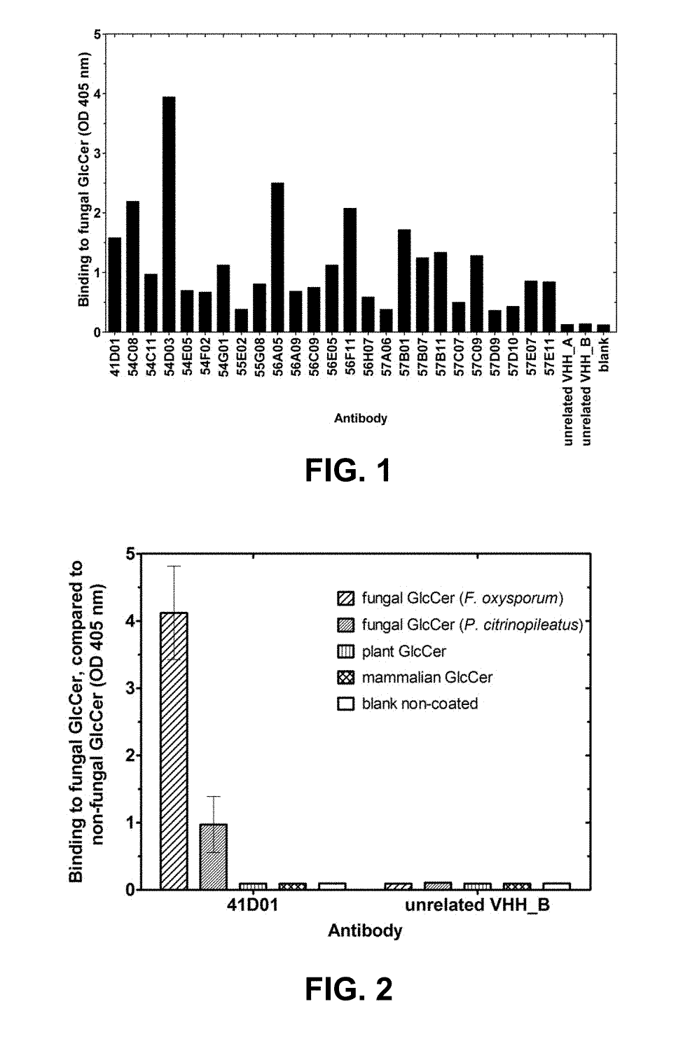

From the primary binding assays 130 VHH-containing periplasmic extracts showed to bind fungal GlcCer with higher OD 405 nm values than the unrelated VHH_A, unrelated VHH_B and blank. OD 405 nm values demonstrating the specific binding of several of these fungal GlcCer binding VHHs are shown in FIG. 1. Sequence analysis revealed 84 unique sequences from the identified set of anti-GlcCer VHH.

Further Characterization by Differential Binding Screens:

For further characterization, VHH belonging to the abovementioned lead panel were produced in E. coli in culture flasks according to standard procedures. Hexahistidine-tagged VHH were purified from the periplasmic extract with TALON metal affinity resin (Clontech), according to the manufacturer's instructions. Purified VHH were concentrated and dialyzed to PBS. VHH were also purified using automated purification systems using a combination of immobilized Nickel IMAC and desalting columns. VHH of the lead panel that scored positively in primary binding assays, were subsequently tested for their specificity towards GlcCer or cell wall fractions from different fungal phytopathogens.

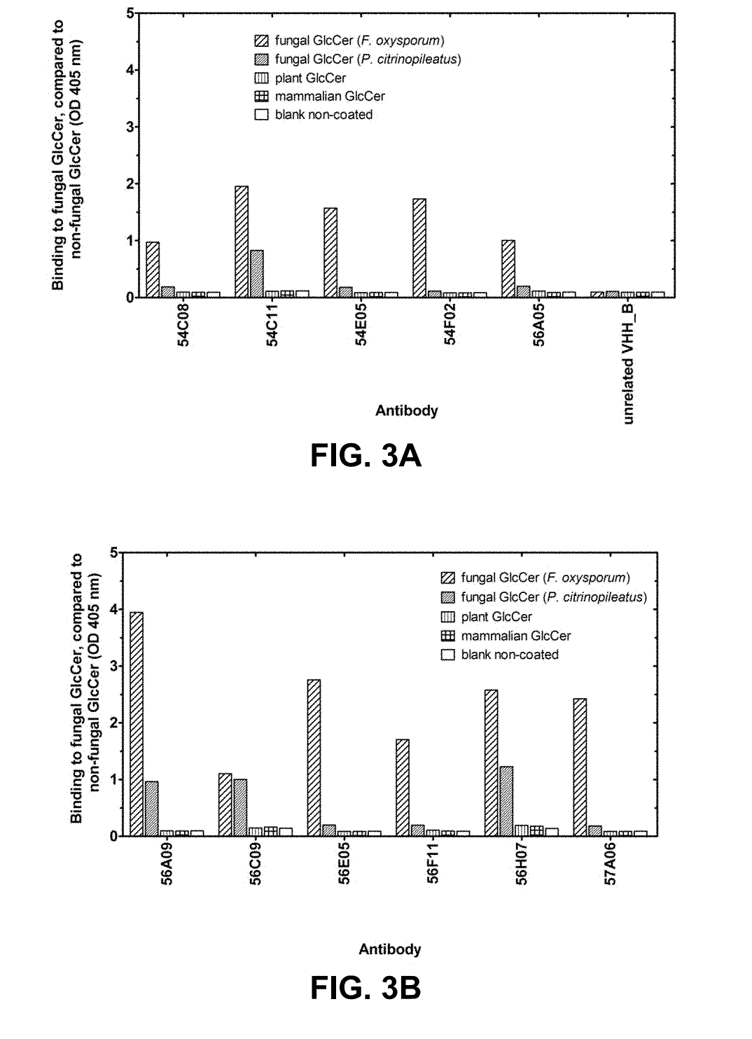

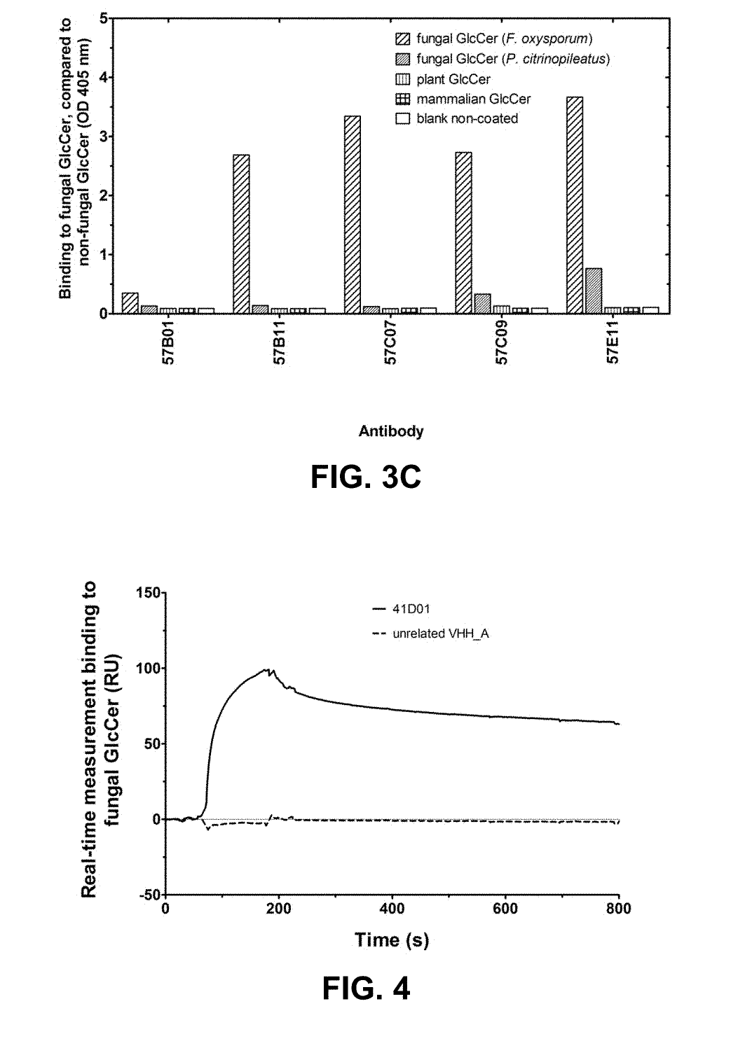

As demonstrated in FIGS. 2, 3A, 3B and 3C, GlcCer-specific VHH showed specific binding to fungal GlcCer (Pleurotus citrinopileatus, Fusarium oxysporum) and not to other non-fungal GlcCer or blank non-coated well.

Surface Plasmon Resonance:

Binding of VHH to fungal GlcCer was characterized by surface plasmon resonance in a BIACORE® 3000 instrument. Anti-GlcCer VHH 41D01 or unrelated VHH_A were covalently bound to CM5 sensor chips surface via amine coupling until an increase of 1000 response units was reached. Remaining reactive groups were inactivated. A range of concentrations of in solution Fusarium oxysporum GlcCer prepared according to Salio et al., 2013 PNAS 110, E4753-E4761 was injected for 2 minutes at a flow rate of 30 μl/minute to allow for binding to chip-bound VHH. Running buffer without GlcCer was injected over the chip at the same flow rate to allow spontaneous dissociation of bound fungal GlcCer for 10 minutes. A K_{off}-value was calculated from the sensorgrams obtained for the different fungal GlcCer concentrations with 1:1 Langmuir dissociation global fitting model.

For anti-GlcCer VHH a slow off-rate of 4.86*1E−4/s was calculated. As shown in FIG. 4, an unrelated VHH did not bind fungal GlcCer.

Plant (soy), mammalian (pork) and fungal (Fusarim oxysporum) GlcCer in solution were sequentially injected for 2 minutes at a flow rate of 30 μl/minute to allow for binding to chip-bound VHH (anti-GlcCer VHH 41D01 or unrelated VHH_A). Running buffer without GlcCer was injected over the chip between each injection at the same flow rate to allow spontaneous dissociation of bound GlcCer.

No plant or mammalian GlcCer binding to anti-GlcCer VHH 41D01 or unrelated VHH_A was observed. Specific binding of fungal GlcCer was observed for anti-GlcCer VHH 41D01 and not for unrelated VHH_A.

Differential Binding to Different Fungal Lipid Extracts:

The binding of anti-GlcCer VHH compositions to different fungal lipid extracts compared to unrelated compounds.

Fungal extracts were prepared according to Rodrigues et al. 2000 Infection and Immunity 68 (12):7049-60. Briefly, mycelium from Botrytis cinerea B05-10, Botrytis cinerea MUCL401, Botrytis cinerea R16, Botrytis cinerea (own pear isolate), Fusarium culmorum MUCL555, Fusarium graminearum MUCL53451, Penicillium digitatum MUCL43-410, Penicillium digitatum (own lemon isolate) or Penicillium expansum CBS 146.45 were harvested from fungi grown in agar plates and lipids were extracted with chloroform/methanol 2:1 (vol/vol) and 1:2 (vol/vol); crude lipid extract was partitioned according to Folch et al. 1957, Journal of Biological Chemistry 226 (1):497-509. Fungal lipid extracts were recovered from Folch's lower phase. Binding of anti-GlcCer VHH 41D01 (0.1 μg/ml) and anti-GlcCer VHH 56F11 (1 μg/ml) was evaluated to wells coated with the extracted fungal lipids (each in 1/20 dilution), purified Fusarium oxysporum GlcCer, purified Pleurotus citrinopileatus GlcCer and unrelated compounds: apple pectin (Apple pectin high esterified 70-75%, Sigma, cat#: 76282), citrus pectin (Citrus pectin low esterified 20-34%, Sigma, cat# P9311) or potato lectin (Solanum Tuberosum Lectin, Vector labs, cat#: L-1160) or a blank non-coated well. Binding was measured after consecutive incubation with enzyme-conjugated detection antibodies, adding substrate, and measuring absorbance at 405 nm. Bars represent average OD 405 nm values, error bars represent standard errors of the mean of n=2.

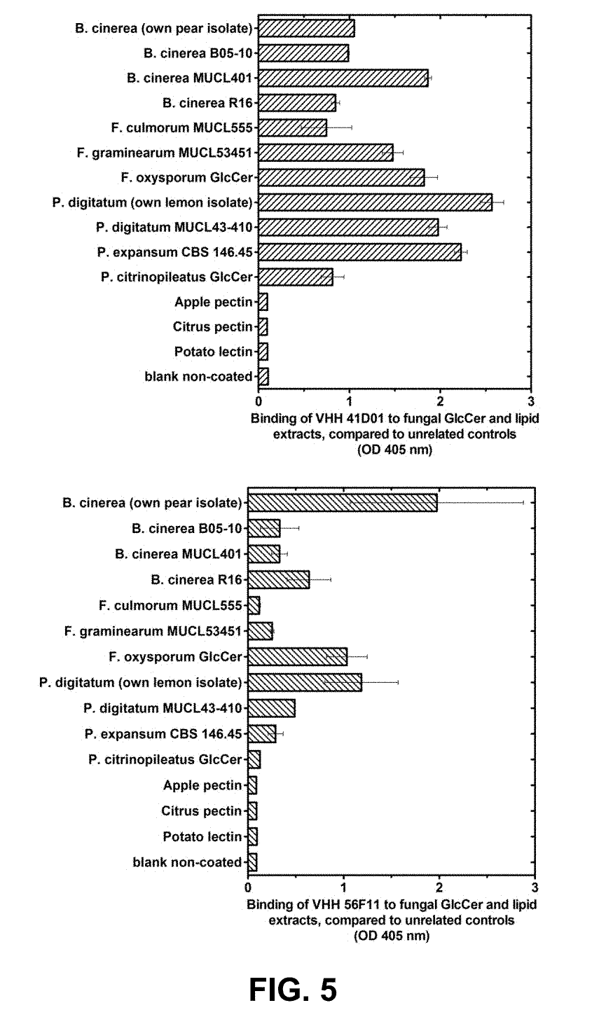

As shown in FIG. 5, anti-GlcCer VHH 41D01 and 56F11 specifically recognized all the fungi lipid extracts tested. Anti-GlcCer VHH 41D01 and 56F11 did not show binding to unrelated coated compounds or non-coated wells. The binding of the anti-GlcCer VHH compositions to a wide array of fungal lipids extracts potentiates a variety of applications for the anti-GlcCer VHH compositions as disclosed herein against different fungi.

Binding of Anti-GlcCer VHH to Fungal GlcCer in Different Aqueous Compositions:

Aqueous compositions containing anti-GlcCer VHH 41D01 and/or protease inhibitors and/or non-ionic surfactants and/or preservatives were prepared. Composition A1 (protease inhibitors: 0.06 μg/ml aprotinin (Roche, cat#: 10236624001), 0.5 μg/ml leupeptin (Roche, cat#: 11017101001), 24 μg/ml 4-benzenesulfonyl fluoride hydrochloride (Sigma, A8456), 1 mM EDTA (Carl-Roth, cat#8040.1) and non-ionic surfactant: 0.00001% Polysorbate 20 (TWEEN®20, Sigma, cat# P2287); Composition A2 (protease inhibitors: 1 μg/ml aprotinin, 2.5 μg/ml leupeptin, 100 μg/ml 4-benzenesulfonyl fluoride hydrochloride, 1 mM EDTA and non-ionic surfactant: 0.05% Polysorbate 20); Composition A3 (protease inhibitors: 2 μg/ml aprotinin, 5 μg/ml leupeptin, 240 μg/ml 4-benzenesulfonyl fluoride hydrochloride, 1 mM EDTA and non-ionic surfactant: 5% Polysorbate 20), Composition B1 (non-ionic surfactant: 0.0001%% Polysorbate 20), Composition B2 (non-ionic surfactant: 0.05% Polysorbate 20), Composition B3 (non-ionic surfactant: 5% Polysorbate 20) and Composition C1 (preservative: 0.05% sodium benzoate (Sigma, cat# B3420)). Binding of anti-GlcCer VHH (at 0.1 μg/ml) to fungal GlcCer in different aqueous compositions was tested in ELISA with coated GlcCer from F. oxysporum and compared to blank non-coated wells. Binding was measured after consecutive incubation with enzyme-conjugated detection antibodies, adding substrate and measuring absorbance at 405 nm.

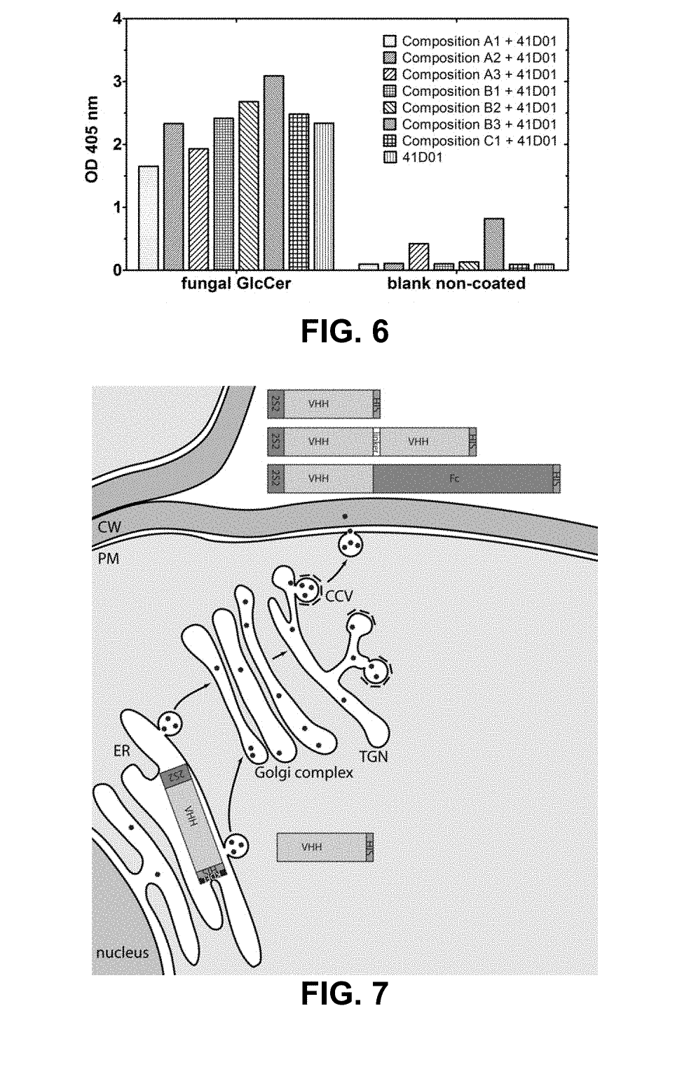

In FIG. 6, values of GlcCer-specific VHH 41D01 in the different compositions were compared with 41D01 in solution without other additives. It is shown in FIG. 6 that GlcCer-specific VHH 41D01 was capable of specifically binding to fungal GlcCer in all tested compositions.

### Example 2

Generation of Transgenic Plants According to Embodiments of this Disclosure

Arabidopsis has been transformed with different vectors for the expression of polynucleotides encoding VHH specifically binding to a sphingolipid of a plant pathogenic fungus. The polynucleotides comprise at least one sequence encoding a targeting signal for secretion (e.g., 2S2), for localization to the cytoplasm (e.g., start codon), or for location to the endoplasmatic reticulum (e.g., KDEL), as schematically illustrated in FIG. 7. The transgenic Arabidopsis plants are analyzed in bioassays with different plant pathogenic fungi.

Transgenic plants comprising a polynucleotide encoding VHH 41D01 were made. Also, transgenic plants comprising a polynucleotide encoding VHH 56F11 were made. The nucleotide sequence of the polynucleotides encoding VHH 41D01 and VHH 56F11 are respectively represented by SEQ ID NO:336 and SEQ ID NO:337.

Different chimeric constructs were made comprising each of the polynucleotides encoding VHH 41D01 and 56F11 and polynucleotides encoding various tag sequences, signal sequences, spacer sequences, hinge sequence, and/or Fc sequences. These different chimeric constructs are listed in Table 4.

TABLE 4
Chimeric constructs for generating transgenic plants according to
embodiments of this disclosure

| Construct name | Targeting | SEQ ID NO |
|---|---|---|
| 41D01_His_KDEL | ER | 338 |
| sec_41D01_hinge_Fc_His | secreted | 339 |
| sec_41D01-9GS-41D01_His | secreted | 340 |
| sec_41D01_His | secreted | 341 |
| cyto_41D01_His | cytoplasmatic | 342 |
| 56F11_His_KDEL | ER | 343 |
| sec_56F11_hinge_Fc_His | secreted | 344 |
| sec_56F11-9GS-56F11_His | secreted | 345 |
| sec_56F11_His | secreted | 346 |
| cyto_56F11_His | cytoplasmatic | 347 |

41D01: VHH1 (SEQ ID NO:336); His: His6 tag, consists of 6 His repeats (SEQ ID NO:348); KDEL: ER retention signal (SEQ ID NO:349); sec: 2S2 seed storage protein gene signal peptide (SEQ ID NO:350); Fc: Fc from mouse IgG3 (SEQ ID NO:351); 9GS: spacer, consisting of GGGGSGGGS (SEQ ID NO:352); hinge: mouse IgG3 hinge (SEQ ID NO:353); cyto: addition of an ATG start codon; 56F11: VHH2 (SEQ ID NO:337); ER: endoplasmatic reticulum.

Table 5 lists the protein sequence of the various tags, signal sequences, spacers, hinge regions, or Fc used to generate the different chimeric constructs.

TABLE 5
Protein sequence of the elements used to generate chimeric constructs

| Sequence elements | SEQ ID NO |
|---|---|
| His6 | 348 |
| ER retention signal | 349 |
| 2S2 seed storage protein gene signal peptide | 350 |
| Fc of mouse IgG3 | 351 |
| 9GS | 352 |
| Mouse IgG3 hinge | 353 |

The chimeric constructs of Table 4 were placed under transcriptional control of the 35S CaMV promoter (SEQ ID NO:354) in the pK7WG2 destination vector (see further below).

### Generation of Entry Vectors

The vectors listed in Table 6 were ordered at GENEART® Gene Synthesis (Life technologies).

TABLE 6
Vectors used to generate entry vectors

| Vector number | Vector name |
|---|---|
| 1 | pMA_56F11-KDEL |
| 2 | pMA_sec-bi56F11 |
| 3 | pMA_sec-56F11Fc |
| 4 | pMA_sec-56F11 |
| 5 | pMA_41D01-KDEL |
| 6 | pMA_sec-bi41D01 |
| 7 | pMA_sec-41D01Fc |
| 8 | pMA_sec-41D01 |

KDEL: 4 amino acid sequence (K, D, E, and L) to retain the expressed VHH in the endoplasmic reticulum (ER); sec: 2S2 signal peptide (to target the expressed VHH to the secretory pathway); bi: indicates that the VHH is expressed as a bivalent; Fc: “fragment crystallizable” chain of the mouse IgG3 antibody

PCR amplification of these sequences was done using specific primers (pMA_FW and pMA_REV) to introduce restriction sites (EcoRI+BamHI). A second set of primers (pCYTO_FW and pMA_REV) was used to introduce a cytoplasmic targeting signal. For this, sequence 4 and 8 were used as a template. The sequences of pMA_FW, pMA_REV, and pCYTO_FW are listed in Table 7.

TABLE 7
Nucleic acid sequences of the primers used
for PCR amplification

| | | SEQ ID |
|---|---|---|
| Primer | Nucleic acid sequence | NO |
| pMA_FW | TTGTAAAACGACGGCCAG | 355 |
| pMA_REV | GGAAACAGCTATGACCATGT | 356 |
| pMA_CYTO | CCGGAATTCCCACCATG | 357 |
| | CAGGTTCAGCTGCAGGAAT | |
| | (underlined section overlaps with | |
| | N-terminal VHH ends) | |

Restriction digest was performed on both amplified fragments and the entry vector E-IgG3-GmR. This vector introduces AttL1 and AttL2 sites for further downstream Gateway-compatible cloning. Digested sequence fragments and vector fragment were purified (using a purification kit for the sequence fragments and in-gel purification for the vector fragment).

Digested sequence fragments were ligated into the digested E-IgG3-GmR vector.

Next, the obtained entry clones (10 in total: 2×VHH-KDEL, 2× sec-VHH, 2× sec-biVHH, 2× sec-VHH-Fc and 2× cyto-VHH) were used for transformation of DH5α E. coli cells.

Per entry clone, 10 colonies were checked via colony-PCR. Of each construct, up to 4 positive clones were plated again, to screen for single colonies. Next, colony-PCR was performed on the generated single colonies using a high-fidelity polymerase (Phusion PCR, New England Biolabs).

The PCR products resulting from this Phusion PCR (35 in total) were sent to LGC Genomics for sequence analysis.

### Generation of Expression Vectors

All 35 entry vectors (representing 10 different entry vector constructs in total) that were sent for sequencing, were initially continued with. DNA was purified of each construct, and used for LR reaction (Gateway cloning) to the pK7WG2 destination vector (Plant Systems Biology (Karimi et al., “Gateway vectors for Agrobacterium-mediated plant transformation,” Trends Plant. Sci. 2002 May; 7 (5): 193-195); SEQ ID NO:358).

The obtained expression clones were used for transformation of DH5α E. coli cells.

Based on the obtained sequencing results from the 35 entry clones, a positive candidate for each of the ten different entry vector constructs was selected.

Per expression clone, two colonies were checked via colony-PCR. From one positive clone per expression clone, plasmid was prepared and sent for sequence analysis (VIB sequence service facility).

A glycerol bank of DH5α E. coli cells containing each of the ten entry clones and each of the ten expression clones was established (20 entries in total). The ten expression vectors are listed in Table 8.

TABLE 8
Expression vectors for generating plants according to embodiments of
this disclosure

| Construct name | Targeting | SEQ ID NO |
|---|---|---|
| pK7WG2-41D01_His_KDEL | ER | 359 |
| pK7WG2-sec_41D01_hinge_Fc_His | secreted | 360 |
| pK7WG2-sec_41D01-9GS-41D01_His | secreted | 361 |
| pK7WG2-sec_41D01_His | secreted | 362 |
| pK7WG2-cyto_41D01_His | cytoplasmatic | 363 |
| pK7WG2-56F11_His_KDEL | ER | 364 |
| pK7WG2-sec_56F11_hinge_Fc_His | secreted | 365 |
| pK7WG2-sec_56F11-9GS-56F11_His | secreted | 366 |
| pK7WG2-sec_56F11_His | secreted | 367 |
| pK7WG2-cyto_56F11_His | cytoplasmatic | 368 |

Transformation of Expression Vectors into Agrobacterium and Arabidopsis Transformation

Each of the 10 expression constructs (SEQ ID NOS:359-368) was transformed into Agrobacterium strain C58C1 Rif^{R}(pMP90); (Koncz and Schell (1986) Mol. Gen. Genet. 204:383-396). Colonies were checked for the presence of the expression vector via colony-PCR. For each of the 10 expression constructs, a positive strain was identified.

Of these positive strains, a glycerol bank was established.

The same positive strains were used for the transformation of Arabidopsis Col-0 WT plants using floral dipping. Five plants were dipped per construct. The floral dip transformation protocol is described in S. J. Clough and A. F. Bent (1998), “Floral dip: a simplified method for Agrobacterium-mediated transformation of Arabidopsis thaliana,” Plant J. 16:735-743.

Floral dip transformation of Arabidopsis generated T1 seeds after 6 weeks. Per construct, 300-400 mg of T1 seed was obtained.

T1 seeds were sterilized by bleach and ethanol, and sown on K1 medium, supplemented with kanamycin (50 mg/L), nystatin (50 mg/L) and vancomycin (750 mg/L). After sowing with 7 ml of 0.1% agarose, plates were first incubated for 4 nights at 4° C., then transferred to the growth chamber. For each construct, 30 kanamycin-resistant T1 plants were transferred to soil and further allowed to set seed.

### VHH Expression Analysis

### T1 Plants—Confirmation of VHH Expression:

VHH Expression Analysis (Protein Level) in Isolated Leaves from T1 Plants by Western Blotting

For each construct (10 in total), VHH protein expression analysis was performed for 30 T1 plants. From each T1 plant, 2 leaves were cut and harvested in 2-ml Eppendorfs, cooled in liquid nitrogen and crushed for 2 minutes at 20 Hz with two steel, 4-mm balls. The powder was dissolved in 100 μl extraction buffer (20 mM Pi, 300 mM NaCl, 0.1% CHAPS, pH 7.8, COMPLETE® protease inhibitor) and centrifuged (10 minutes, max speed, 4° C.) to spin down the cell debris. The supernatant was transferred to a fresh Eppendorf and centrifuged again (10 minutes, max speed, 4° C.). Ninety μl of final supernatant was kept, 22.5 μl of glycerol was added and the extract was stored at −20° C. Total protein content of each extract was determined by Bradford analysis. The total protein concentrations ranged from 0.5 to 4 mg/ml.

Protein extracts were analyzed by SDS-PAGE (12% TGX gels; Bio-Rad) followed by Western blotting (WB). For SDS-PAGE analysis, a volume corresponding with 10 μg of total protein content of each extract was loaded onto gel. WB detection was performed in 2 steps: the primary antibody was a mouse anti-His (Serotec; 1/1000 dilution). The secondary antibody comprised a mixture of 2 antibodies: sheep anti-mIgG-HRP (GE; 1/5000 dilution) and anti-IgG1-HRP (Sigma; 1/1000 dilution). For the constructs that contain the Fc fragment, detection was performed in a single step using goat anti-mouse IgG3-HRP (Sigma; 1/5000 dilution). All events for which a band appeared that corresponded with the appropriate size of the VHH-construct were considered as events in which the VHH-construct was expressed.

From the WB analysis, it became clear that expression was highest for VHHs fused to an Fc fragment. It was decided to continue analysis with the constructs sec_41D01_hinge_Fc_HIS and sec_56F11_hinge_Fc_HIS.

T1 plants that showed expression of VHH on WB were further allowed to set seed and T2 seed were harvested for segregation analysis.

### T2 Plants—Segregation Analysis

T2 seeds were sterilized by bleach and ethanol, and sown on K1 medium, supplemented with kanamycin (50 mg/L), nystatin (50 mg/L) and vancomycin (750 mg/L). After sowing with 7 ml of 0.1% agarose, plates were first incubated for 4 nights at 4° C., and then transferred to the growth chamber. To identify lines containing the expression construct at a single locus in the genome, plates were scored for the ratio of the number of kanamycin-sensitive to resistant seedlings, and plates for which this ratio diverged from 1:3 were discarded. For the single-copy lines that showed the highest VHH expression, 10 plants were transferred to soil and further allowed to set seed. T3 seed were harvested for zygosity analysis.

### T3 Plants—Zygosity Analysis

T3 seeds were sterilized by bleach and ethanol, and sown on K1 medium, supplemented with kanamycin (50 mg/L), nystatin (50 mg/L) and vancomycin (750 mg/L). After sowing with 7 ml of 0.1% agarose, plates were first incubated for 4 nights at 4° C., and then transferred to the growth chamber. Plates were scored for the number of kanamycin-resistant seedlings, and plates for which not all seedlings were resistant were discarded. For the remaining homozygous lines, plants were transferred to soil for seed propagation.

### Example 3

### Functional Assays

### Glucosylceramide Binding Assay

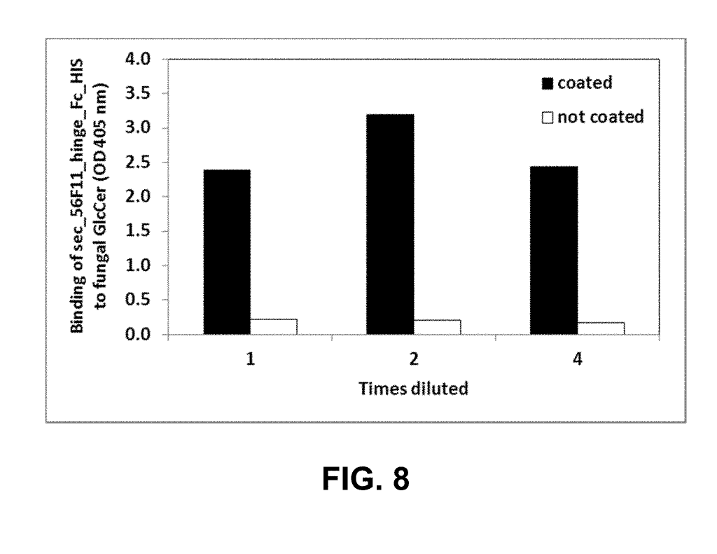

Leaf extract from a homozygous single-copy event of Arabidopsis overexpressing sec_56F11_hinge_Fc_HIS was tested in an ELISA for binding of glucosylceramide (GlcCer). For this, wells of a multi-well plate (Greiner Bio-one, μClear, black, half area, high bind) was coated with 250 ng (50 μl of a 5 μg/ml solution) of GlcCer purified from Fusarium oxysporum. After coating, the plate was blocked with 1% gelatin in PBS for 1 hour. The blocking agent was removed and the plate incubated with 50 μl of (diluted) leaf extract for 1 hour. Next, the plate is washed 3 times with PBS. The plate is incubated with mouse anti-His (Serotec) for 1 hour. After washing the plate 3 times with PBS, the plate was incubated with anti-mouse IgG/alkaline phosphatase antibody (Sigma) for 1 hour. After washing three times with PBS, the plate was developed by adding ELISA buffer (100 mM Tris-HCl; 100 mM NaCl; 5 mM MgCl; pH 9.5; containing 2 mg/ml PNPP ELISA substrate (Sigma)) in each well. After 5 minutes, the absorbance at 405 nm was measured. As a control, uncoated wells were used. FIG. 8 shows the specific binding of sec_56F11_hinge_Fc_HIS in leaf extract to fungal GlcCer.

Botrytis cinerea Infection Assay

A. thaliana wild-type (Col-0) and plants overexpressing sec_41D01_hinge_Fc_HIS and sec_56F11_hinge_Fc_HIS were grown for five weeks in soil (“DCM potgrond voor Zaaien en Stekken,” DCM, Sint-Katelijne-Waver, Belgium) in a growth chamber with 21° C., 75% humidity and a 12-hour day-light cycle with a light intensity of approximately 120 μmol/m²s. A 5 μL drop of a B. cinerea spore suspension (B05.10, 5×10⁴/mL in ½ PDB) was inoculated onto three leaves per plant. Plants were kept in transparent sealed boxes to retain almost 100% humidity after inoculation. Disease symptoms were scored by measuring the diameters of the necrotic lesions on 3, 4 and 7 days post inoculation (dpi). Twenty plants per line and condition, divided over 5 boxes, were analyzed.

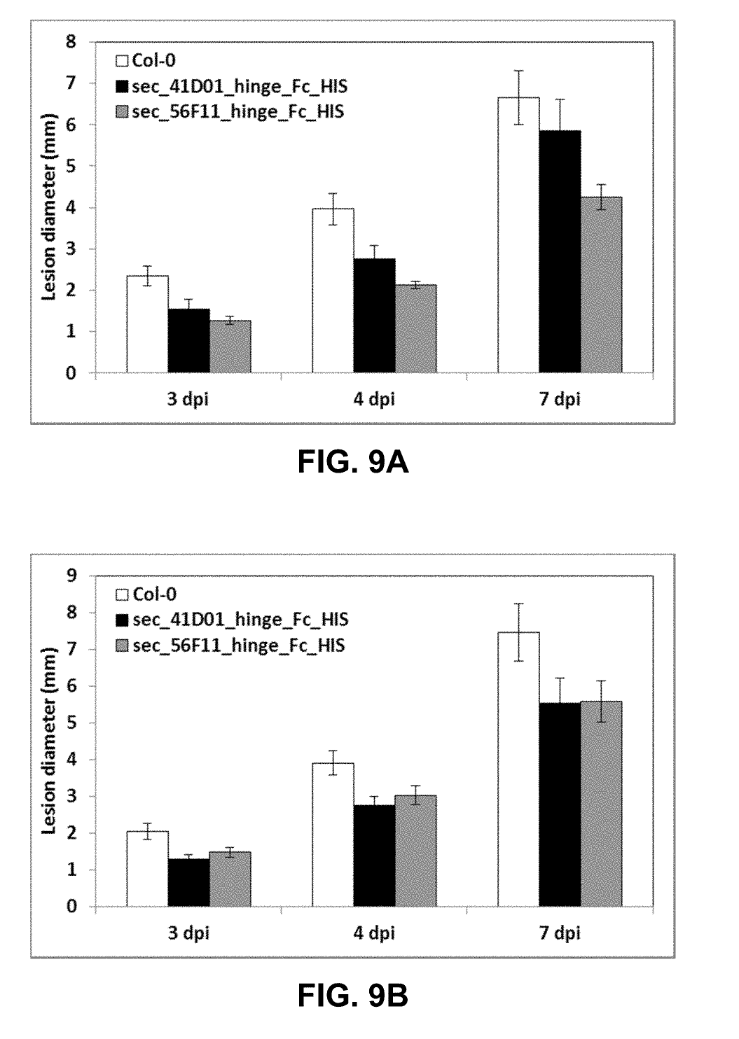

FIG. 9A shows increased resistance against B. cinerea in plants expressing VHH against fungal GlcCer as compared to wild-type plants.

A further infection assay was performed under identical conditions using twenty four plants per line and condition, divided over 6 boxes. A different line of plants overexpressing sec_41D01_hinge_Fc_HIS was used than in the first bioassay. FIG. 9B confirms increased resistance against B. cinerea in plants expressing VHH against fungal GlcCer as compared to wild-type plants.

### Example 4

### In Vitro Evaluation of the Antifungal Activity of Anti-GlcCer VHH-Containing Leaf Extract

From a homozygous single-copy event of Arabidopsis overexpressing anti-GlcCer VHH, 2 leaves are cut and harvested in 2-ml Eppendorfs, cooled in liquid nitrogen and crushed for 2 minutes at 20 Hz with two steel, 4-mm balls. The powder is dissolved in 100 μl extraction buffer (20 mM Pi, 300 mM NaCl, 0.1% CHAPS, pH 7.8, COMPLETE® protease inhibitor) and centrifuged (10 minutes, max speed, 4° C.) to spin down the cell debris. The supernatant is transferred to a fresh Eppendorf and centrifuged again (10 minutes, max speed, 4° C.). This final supernatant is used in the antifungal bioassay.

The antifungal activity of anti-GlcCer VHH-containing leaf extract is tested using antifungal assays in liquid media and on agar plates as described in Thevissen et al., 2011, Bioorg. Med. Chem. Lett. 21 (12):3686-92; Frangois et al., 2009, J. Biol. Chem. 284 (47):32680-5; Aerts et al., 2009, FEBS Lett. 583 (15):25143-6. The inhibitory action is determined for the anti-GlcCer VHH-containing leaf extract on in vitro growth of Botrytis cinerea and Phytophthora infestans.

Two-fold dilutions of the anti-GlcCer VHH-containing leaf extract in water are prepared in 96-well microtiter plates. To 20 μl of these dilutions and to 20 μl of water as a control, 80 μl of fungal spores suspension (1E+05 spores/ml in half strength potato dextrose broth (PDB)) are added. The fungal test strains are Alternaria brassicicola MUCL20297, Botrytis cinerea R16, Cercospora beticola (own sugar beet isolate), Fusarium culmorum MUCL555 and Verticillium dahliae MUCL6963. The test plates are incubated for 72 hours at room temperature in the dark and the antifungal activity of the test compounds is scored microscopically and quantified based on photographic standards, whereby a score of 0 or 100 refers to no or maximal fungal growth, respectively. All tests are performed in at least 2 replicas.

The results of the antifungal activity assays, indicate a clear difference between the growth inhibition pattern, expressed as the % fungal growth in function of dilution of the anti-GlcCer VHH-containing leaf extract. This difference is clear irrespective of the species of the test fungus.

The results show the antifungal potency of anti-GlcCer VHH-containing leaf extract. Moreover, the results reveal a broad-spectrum of antifungal activity of anti-GlcCer VHH-containing leaf extract towards at least 5 different fungal plant pathogens and indicate that the spectrum of antifungal activity of the selected anti-GlcCer VHH-containing leaf extract can be broadened to other plant pathogenic fungi.

### Example 5

### In Planta Evaluation of the Antifungal Activity of Leaf Extracts Containing Anti-GlcCer VHH to Protect Crops Against Fungal Infection

Efficacy of leaf extracts containing anti-GlcCer VHH on tomato leaves inoculated with Botrytis cinerea: preventive treatment.

Leaf extract from homozygous single-copy event of Arabidopsis overexpressing anti-GlcCer VHH is tested in a Botrytis cinerea infection assay on tomato. The effect of a preventive treatment with plant extracts containing anti-GlcCer VHH on the disease severity of Botrytis cinerea B05-10 inoculated tomato leaves is evaluated and compared with the effect of water.

Leaves from greenhouse grown tomato plants are treated with 10 μl of VHH-containing leaf extract and water. Upon drying of the applied compositions, 10 μl drops of a Botrytis cinerea spores suspension (6E+06 spores/ml in 4-fold diluted PDB) are applied on the treated surfaces. Treated and inoculated leaves are incubated at high relative humidity and at room temperature in small plant propagators. Disease severity is scored measuring the bidirectional diameter at 6 days post inoculation (dpi).

Preventive treatment with the anti-GlcCer VHH composition results in a smaller average lesion diameter than treatment with water. Preventive treatment of tomato leaves with the application of the anti-GlcCer VHH-containing leaf extract clearly results in a reduction of disease severity compared with the treatment with water. Therefore, anti-GlcCer VHH-containing leaf extracts show the potency to be used as antifungal compounds to protect crops against fungal pathogens in agricultural applications.

Efficacy of Anti-GlcCer VHH Compositions on Tomato Leaves Inoculated with Botrytis cinerea: Curative Treatment.

The effect of a curative treatment with anti-GlcCer VHH-containing leaf extract on the disease severity of Botrytis cinerea B05-10 inoculated tomato leaves is evaluated and compared with the effect of water.

Leaves from greenhouse-grown tomato plants are inoculated with 10 μl drops of a Botrytis cinerea spores suspension ((6E+06 spores/ml) in 4-fold diluted PDB). One hour after inoculation, the inoculated spots on the leaves are treated with 10 μl of VHH-containing leaf extract and water. Inoculated and treated leaves are incubated at high relative humidity and at room temperature in small plant propagators. Disease severity is scored measuring the bidirectional diameter at 5 dpi.

Curative treatment with anti-GlcCer VHH-containing leaf extract results in a smaller average lesion diameter than treatment with water. Curative treatment of tomato leaves with the application of anti-GlcCer VHH-containing leaf extract clearly results in a reduction of disease severity compared with the treatment with water Therefore, anti-GlcCer VHH-containing leaf extracts show the potency to be used as antifungal compounds to protect crops against fungal pathogens in agricultural applications.

What is claimed is:

1. A transgenic plant or plant tissue or plant cell comprising:
at least one polynucleotide comprising at least one sequence encoding a variable domain of a heavy-chain antibody (VHH) that specifically binds to a sphingolipid of a fungus.

2. The transgenic plant or plant tissue or plant cell according to claim 1, wherein the expression of the polynucleotide in at least part of the transgenic plant or plant tissue or plant cell (i) protects at least part of the transgenic plant or plant tissue or plant cell from an infection with a plant pathogenic fungus, (ii) inhibits the growth of a plant pathogenic fungus on at least part of the transgenic plant or plant tissue or plant cell, and/or (iii) increases the resistance of at least part of the transgenic plant or plant tissue or plant cell against a plant pathogenic fungus.

3. The transgenic plant or plant tissue or plant cell according to claim 1, wherein the polynucleotide comprises a promoter suitable for expression in plants, a plant tissue or plant cell specific promoter, or an inducible promoter.

4. The transgenic plant or plant tissue or plant cell of claim 1, wherein the polynucleotide comprises at least one sequence encoding a targeting signal for secretion, for location to the cytoplasm, or for location to cellular compartments or organelles.

5. The transgenic plant or plant tissue or plant cell of claim 1, wherein the polynucleotide encodes the VHH as such, as a combination with one or more identical or different VHHs, or as a combination with one or more identical or different VHHs with a fragment crystallizable region (Fc region); optionally with a spacer.

6. The transgenic plant or plant tissue or plant cell of claim 1, wherein the plant is a plant selected from the group consisting of corn, rice, wheat, barley, sorghum, millet oats, rye, triticale or other cereals, soybean, alfalfa or other leguminous crops, sugar beet, fodder beet, papaya, banana and plantains or other fruits, grapevines, nuts, oilseed rape, sunflower or other oil crops, squash cucumber, melons or other cucurbits, cotton or other fiber plants, sugarcane, palm, jatropha or other fuel crops, cabbages, tomato, pepper or other vegetables, ornamentals, shrubs, poplar, eucalyptus or other trees, evergreens, grasses, coffee plants, tea plants, tobacco plants, hop plants, rubber plants, and latex plants.

7. The transgenic plant or plant tissue or plant cell of claim 1, wherein the polynucleotide comprises a sequence encoding a VHH comprising:
(i) any one or more of SEQ ID NOS:1 to 84, and/or
(ii) a CDR1, CDR2, and CDR3 region, wherein (i) the CDR1 region is selected from the group of SEQ ID NOS:85-168, and/or (ii) the CDR2 region is selected from the group of SEQ ID NO:169-252, and/or (iii) the CDR3 region is selected from the group of SEQ ID NOS:253-335, or the CDR3 region has the amino acid sequence NRY.

8. Harvestable parts and propagation materials of the transgenic plant or plant tissue or plant cell of claim 1, the harvestable parts and propagation materials comprising:
at least one polynucleotide comprising at least one polynucleotide encoding a variable domain of a heavy-chain antibody (VHH) that specifically binds to a sphingolipid of a fungus.

9. The harvestable parts and propagation materials according to claim 8, wherein the harvestable parts and propagation materials of a transgenic plant or plant tissue or plant cell are selected from the group consisting of seeds, fruits, grains, bulbs, bolls, tubers, progeny, and hybrids.

10. A method for producing the transgenic plant or plant tissue or plant cell of claim 1, the method comprising:
introducing at least one polynucleotide comprising at least one polynucleotide encoding a variable domain of a heavy-chain antibody (VHH) that specifically binds to a sphingolipid of a fungus into the genome of a plant or plant tissue.

11. A method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, the method comprising:
expressing in at least part of the plant or plant tissue or plant cell at least one polynucleotide encoding a variable domain of a heavy-chain antibody (VHH) specifically binding to a pathogen.

12. The method according to claim 11, for protecting at least part of a plant or plant tissue or plant cell from an infection with a plant pathogenic fungus, for inhibiting the growth of a plant pathogenic fungus on at least part of a plant or plant tissue or plant cell, and/or for increasing resistance of at least part of a plant or plant tissue or plant cell against the plant pathogenic fungus, wherein the method comprises:
expressing in at least part of the plant or plant tissue or plant cell at least one polynucleotide comprising at least one polynucleotide encoding a VHH that specifically binds to a sphingolipid of a fungus.

13. An extract of the transgenic plant or plant tissue or plant cell of claim 1, said extract comprising said VHH.

14. A composition comprising the extract of claim 13.

15. A method for protecting at least part of a plant or plant tissue or plant cell from an infection with a plant pathogen, for inhibiting the growth of a plant pathogen on at least part of a plant or plant tissue or plant cell, and/or for increasing pathogen resistance of at least part of a plant or plant tissue or plant cell, the method comprising:
treating said at least part of a plant or plant tissue or plant cell with the extract of claim 13.

16. The transgenic plant or plant tissue or plant cell of claim 1, wherein the VHH specifically binds to glucocerebroside of a fungus.

17. The transgenic plant or plant tissue or plant cell of claim 4, wherein the polynucleotide comprises at least one sequence encoding a targeting signal for location to a cellular part selected from the group consisting of the endoplasmatic reticulum (ER) lumen, the apoplast, the vacuole, intra-membranes, exterior membranes, and a combination of any thereof.

18. The transgenic plant or plant tissue or plant cell of claim 7, wherein the polynucleotide comprises a sequence encoding a VHH comprising SEQ ID NO:1.

19. The transgenic plant or plant tissue or plant cell of claim 7, wherein the polynucleotide comprises a sequence encoding a VHH comprising SEQ ID NO:2.

20. The transgenic plant or plant tissue or plant cell of claim 7, wherein the polynucleotide comprises a sequence encoding a VHH comprising SEQ ID NO:70.


SEQUENCE LISTING (368 sequences)

SEQ ID NO: 1 - protein, 115 aa, Artificial Sequence
  VHH sequence 41D01
QVQLQESGGGLVQAGGSLRLSCAASGRTFSRYGMGWFRQLPGKQRELVTSITRGGTTTYADSVKGRFTISRDNAKNTVYLQMNSLKPEDTAVYYCNARSIWRDYWGQGTQVTVSS

SEQ ID NO: 2 - protein, 121 aa, Artificial Sequence
  VHH sequence 56F11
QVQLQESGGGLVQSGGSLRLSCVHSKTTFTRNAMGWYRQALGKERELVATITSGGTTNYADSVKGRFTISMDSAKNTVYLQMNSLKPEDTAVYYCNVNTRRIFGGTVREYWGQGTQVTVSS

SEQ ID NO: 3 - protein, 118 aa, Artificial Sequence
  VHH sequence 40F07
QVQLQESGGGLVQAGGSLRLSCVASGTTFSSYTMGWYRQAPGKQRELLASIEGGGNTDYADSVKGRFTISRDNARNTVYLQMNSLKTEDTAVYYCNAARTWSIFRNYWGQGTQVTVSS

SEQ ID NO: 4 - protein, 116 aa, Artificial Sequence
  VHH sequence 41D06
QVQLQESGGGLVQAGGSLRLSCAASGGIFGINAMRWYRQAPGKQRELVASISSGGNTNYSESVKGRFTISRDDANYTVYLQMNSLKPEDTAVYYCNFVRLWFPDYWGQGTQVTVSS

SEQ ID NO: 5 - protein, 121 aa, Artificial Sequence
  VHH sequence 41G10
QVQLQESGGGLVQPGGSLTLSCAATKTGFSINAMGWYRQAPGKQREMVATITSGGTTNYADSVKGRFAISRDNAKNTVSLQMNTLKPEDTALYYCNTEARRYFTRASQVYWGQGTQVTVSS

SEQ ID NO: 6 - protein, 120 aa, Artificial Sequence
  VHH sequence 41H05
QVQLQESGGGLVQPGGSLRLSCAASGGIFSINAMGWYRQDPGKQREMVATITSGANTNYTDSVKGRFTISRDNAKNTVYLQMNSLKPEDTAVYYCNAVGRRWYGGYVELWGQGTQVTVSS

SEQ ID NO: 7 - protein, 119 aa, Artificial Sequence
  VHH sequence 42C11
QVQLQESGGGLVQPGGSLRLSCAASGSIFSTYVMGWYRQAIGKQRELVATITSSGKTNYAASVKGRFTVSRDITKNTMYLQMNSLKPEDTAVYYCGADRWVLTRWSNYWGQGTQVTVSS

SEQ ID NO: 8 - protein, 116 aa, Artificial Sequence
  VHH sequence 42C12
QVQLQESGGGLVQPGGSLRLSCAASGSISSLGWYRQAPGKQREFVASATSGGDTTYADSVKGRFTISRDNSKNTVYLQMNSLKPEDTAVYYCKGQRGVAWTRKEYWGQGTQVTVSS

SEQ ID NO: 9 - protein, 119 aa, Artificial Sequence
  VHH sequence 50D03
QVQLQESGGGLVQPGGSLRLSCAASGSIFSTYAMGWYRQAIGKQRELVATITSSGKTNYAASVKGRFTISRDITKNTMYLQMNSLKPEDTAVYYCGADRWVLTRWSNYWGQGTQVTVSS

SEQ ID NO: 10 - protein, 120 aa, Artificial Sequence
  VHH sequence 50D07
QVQLQESGGGLVQPGGSLRLSCTASGNIVNIRDMGWYRQVPGKQRELVATITSDQSTNYADSVKGRFTTTRDNAKKTVYLQMDSLKPEDTAGYYCNARVRTVLRGWRDYWGQGTQVTVSS

SEQ ID NO: 11 - protein, 119 aa, Artificial Sequence
  VHH sequence 50E02
QVQLQESGGGLVQPGGSLRLSCAASGSIFSINAMGWYRQAPGKQRELVAAITSDGSTNYADSVKGRFTISRDNAKNTAYLQMNSLKPEDTAVYYCNLRRRTFLKSSDYWGQGTQVTVSS

SEQ ID NO: 12 - protein, 123 aa, Artificial Sequence
  VHH sequence 51B08
QVQLQESGGGLVQAGDSLRLSCAASGRRFGSYAMGWFRQVPGKERELVAGISSGGSTKYADSVRGRFTISRDNAKNTVSLQMKSLKPEDTAVYYCNAKYGRWTYTGRPEYDSWGQGTQVTVSS

SEQ ID NO: 13 - protein, 116 aa, Artificial Sequence
  VHH sequence 51C06
QVQLQESGGGLVQPGGSLRLSCAASGSIFSSDTMGWYRRAPGKQRELVAAITTGGNTNYADSVKGRFTISRDNAKNTVYLQMNSLQPEDTAVYYCNCRRRWSRDFWGQGTQVTVSS

SEQ ID NO: 14 - protein, 119 aa, Artificial Sequence
  VHH sequence 51C08
QVQLQESGGGLVQPGGSLRLSCAASGTIFSIKTMGWYRQAPGKQRELVATISNGGSTNYADSVKGRFTISRDNAKNTVYLQMNSLKPEDTAVYYCNARQQFIGAPYEYWGQGTQVTVSS

SEQ ID NO: 15 - protein, 115 aa, Artificial Sequence
  VHH sequence 52A01
QVQLQESGGGLVQAGGSLRLSCTASGAITFSLGTMGWYRQAPGKQRELVASISTGSTNYADSVKGRFTISRDIIKNILYLQMNSLKPEDTAVYSCNARLLWSNYWGQGTQVTVSS

SEQ ID NO: 16 - protein, 117 aa, Artificial Sequence
  VHH sequence 52B01
QVQLQESGGGLVQAGESLRLSCAASGSTFSINVMGWYRQAPGEQRELVATISRGGSTNYADSVKGRFTISRDNAKVTVYLQMDSLKPEDTAVYYCNAAGWVGVTNYWGQGTQVTVSS

SEQ ID NO: 17 - protein, 117 aa, Artificial Sequence
  VHH sequence 52G05
QVQLQESGGGLVQAGGSLRLSCAASGSTGSISAMGWYRQAPGKQRELVASITRRGSTNYADSVKDRFTISRDNAWNTVYLQMNSLKPEDTAVYYCNARRYYTRNDYWGQGTQVTVSS

SEQ ID NO: 18 - protein, 114 aa, Artificial Sequence
  VHH sequence 53A01
QVQLQESGGGLGQAGGSLRLSCEVSGTTFSINTMGWHRQAPGKQRELVASISSGGWTNYADSVKGRFTISRDNAKKTVYLQMNNLKPEDTAVYYCRWGAIGNWYGQGTQVTVSS

SEQ ID NO: 19 - protein, 117 aa, Artificial Sequence
  VHH sequence 53F05
QVQLQESGGGLVQPGGSLRLSCAASVRIFGLNAMGWYRQGPGKQRELVASITTGGSTNYAEPVKGRFTISRDNANNTVYLQMNNLKPEDTAVYYCNAERRWGLPNYWGQGTQVTVSS

SEQ ID NO: 20 - protein, 126 aa, Artificial Sequence
  VHH sequence 54A02
QVQLQESGGGLVEAGGSLRLSCAASGRTFSRYGMGWFRQAPGKEREFVAANRWSGGSTYYADSVRGRFTISRDNAKNTVYLQMNSLKPEDTAVYYCAAYAHITAWGMRNDYEYDYWGQGTQVTVSS

SEQ ID NO: 21 - protein, 125 aa, Artificial Sequence
  VHH sequence 54B01
QVQLQESGGGLVQAGGSLRLSCAATGRTFSRYTMGWFRQAPGKERDFVAGITWTGGSTDYADSVKGRFTISRDNAKNTVYLQMNSLKPEDTAVYYCAAGNLLRLAGQLRRGYDSWGQGTQVTVSS

SEQ ID NO: 22 - protein, 129 aa, Artificial Sequence
  VHH sequence 54C01
QVQLQESGGGLVQAGGSLRLSCAASGRTGSRYAMGWFRQAPGKEREFVAAISWSGGSTYYADSVKDRFTISRDNAKNTVYLQMHSLKPEDTAVYYCATRNRAGPHYSRGYTAGQEYDYWGQGTQVTVSS

SEQ ID NO: 23 - protein, 121 aa, Artificial Sequence
  VHH sequence 54C04
QVQLQESGGGLVQPGGSLRLSCAASGRIFSINAMGWYRQGPGKERELVVDMTSGGSINYADSVSGRFTISRDNAKNTVYLQMNSLKPEDTAVYYCHANLRTAFWRNGNDYWGQGTQVTVSS

SEQ ID NO: 24 - protein, 115 aa, Artificial Sequence
  VHH sequence 54C08
QVQLQESGGGLVQPGGSLRLSCAASGSISSINAMGWYRQAPGKQRELVASITSGGSTNYADSVKGRFTISRDNAKNTVNLQMNSLKPEDTAVYYCSAGPWYRRSWGRGTQVTVSS

SEQ ID NO: 25 - protein, 116 aa, Artificial Sequence
  VHH sequence 54C10
QVQLQESGGGLVQPGESLRLSCAASASIFWVNDMGWYRQAPGKQRELVAQITRRGSTNYADSVKGRFTISRDNAKDEVYLQMNSLKPEDTAVYYCNADLAVRGRYWGQGTQVTVSS

SEQ ID NO: 26 - protein, 119 aa, Artificial Sequence
  VHH sequence 54C11
QVQLQESGGGLVQPGGSLRLSCAASGSFFPVNDMAWYRQALGNERELVANITRGGSTNYADSVKGRFTISRDNAKNTVYLQMNTLKPEDTAVYYCNVRIGFGWTAKAYWGQGTQVTVSS

SEQ ID NO: 27 - protein, 116 aa, Artificial Sequence
  VHH sequence 54D03
QVQLQESGGGLVQPGGSLRLSCAASGGIFGINAMRWYRQAPGKQRELVASISSGGNTNYSESVKGRFTISRDDANYTVYLQMNSLKPEDTAVYYCNFVRLWFPDYWGQGTQVTVSS

SEQ ID NO: 28 - protein, 116 aa, Artificial Sequence
  VHH sequence 54D06
QVQLQESGGGLVQPGGSLRLSCAASGSTIRINAMGWYRQAPGKQRELVATITRGGITNYADSVKGRFTISRDNAKFTVYLQMNSLKPEDTAVYYCNARSWVGPEYWGQGTQVTVSS

SEQ ID NO: 29 - protein, 120 aa, Artificial Sequence
  VHH sequence 54D10
QVQLQESGGGLVQPGGSLRLSCAASGMTYSIHAMGWYRQAPGKERELVAITSTSGTTDYTDSVKGRFTISRDGANNTVYLQMNSLKSEDTAVYYCHVKTRTWYNGKYDYWGQGTQVTVSS

SEQ ID NO: 30 - protein, 117 aa, Artificial Sequence
  VHH sequence 54E01
QVQLQESGGGLVQPGGSLRLSCTASGSIFSINPMGWYRQAPGKQRELVAAITSGGSTNYADYVKGRFTISRDNAKNVVYLQMNSLKPEDTAVYYCNGRSTLWRRDYWGQGTQVTVSS

SEQ ID NO: 31 - protein, 117 aa, Artificial Sequence
  VHH sequence 54E05
QVQLQESGGGLVQPGGSLRLSCAASGSIFSINTMGWYRQAPGKQRELVAAITNRGSTNYADFVKGRFTISRDNAKNTVYLQMNSLKPDDTAVYYCNAHRSWPRYDSWGQGTQVTVSS

SEQ ID NO: 32 - protein, 118 aa, Artificial Sequence
  VHH sequence 54E10
QVQLQESGGGLVQPGGSLRLSCAASGSIFSFNAMGWYRQAPGKQRELVAAITRGGSTNYADSVKGRFTISRDNANNTVYLQMNSLKPEDTAVYYCNAESRIFRRYDYWGPGTQVTVSS

SEQ ID NO: 33 - protein, 115 aa, Artificial Sequence
  VHH sequence 54F01
QVQLQESGGGLVQPGGSLRLSCVTSGSIFGLNLMGWYRQAPGKQRELVATITRGGSTNYADSVKGRFTISRDNAKKTVYLQMNSLKPEDTAVYYCNVDRGWSSYWGQGTQVTVSS

SEQ ID NO: 34 - protein, 119 aa, Artificial Sequence
  VHH sequence 54F02
QVQLQESGGGLVQPGGSLRLSCVTSGSIRSINTMGWYRQAPGNERELVATITSGGTTNYADSVKNRFTISRDNAKNTVYLQMNSLKPEDTAVYYCNLHQRAWARSYVYWGQGTQVTVSS

SEQ ID NO: 35 - protein, 117 aa, Artificial Sequence
  VHH sequence 54G01
QVQLQESGGGSVQPGGSLRLSCAASGSIFAVNAMGWYRQAPGHQRELVAIISSNSTSNYADSVKGRFTISRDNAKNTVYLQMNSLKPEDTAVYFCYAKRSWFSQEYWGQGTQVTVSS

SEQ ID NO: 36 - protein, 121 aa, Artificial Sequence
  VHH sequence 54G08
QVQLQESGGGLVQPGGSLRLSCAASGSIFSFNLMGWYRQAPGKQRELVAAITSSSNTNYADSVKGRFTISRDNAKNTVYLQMNSLKPEDTAVYYCNAQYTITPWGIKKDYWGQGTQVTVSS

SEQ ID NO: 37 - protein, 120 aa, Artificial Sequence
  VHH sequence 54G09
QVQLQESGGGLMQPGGSLRLSCTASGNIVNIRDMGWYRQVPGKQRELVATITSDQSTNYADSVKGRFTTTRDNAKKTVYLQMDSLKPEDTAGYYCNARVRTVLRGWRDYWGQGTQVTVSS

SEQ ID NO: 38 - protein, 117 aa, Artificial Sequence
  VHH sequence 55B02
QVQLQESGGGLVQPGESLRLSCVGSGSIFNINSMNWYRQASGKQRELVADMRSDGSTNYADSVKGRFTISRDNARKTVYLQMNSLKPEDTAVYYCHANSIFRSRDYWGQGTQVTVSS

SEQ ID NO: 39 - protein, 125 aa, Artificial Sequence
  VHH sequence 55B05
QVQLQESGGGVVQAGDSLRLSCAASGRTFGGYTVAWFRQAPGKEREFVARISWSGIMAYYAESVKGRFTISRDNAKNTVYLQMNSLKPEDTAVYYCASRSQIRSPWSSLDDYDRWGQGTQVTVSS

SEQ ID NO: 40 - protein, 116 aa, Artificial Sequence
  VHH sequence 55C05
QVQLQESGGGLVQPGGSLRLSCVVSGSISSMKAMGWHRQAPGKERELVAQITRGDSTNYADSVKGRFTISRDNAKNTVYLQMNSLKPDDTGVYYCNADRFFGRDYWGKGTQVTVSS

SEQ ID NO: 41 - protein, 120 aa, Artificial Sequence
  VHH sequence 55D08
QVQLQESGGGLVQPGGSLRLSCAASRSILSISAMGWYRQGPGKQREPVATITSAGSSNYSDSVKGRFTISRDNAKNTAYLQMNSLKPEDTAVYYCKTVYSRPLLGPLEVWGQGTQVTVSS

SEQ ID NO: 42 - protein, 115 aa, Artificial Sequence
  VHH sequence 55E02
QVQLQESGGGLVQTGGSLRLSCVASGSMFSSNAMAWYRQAPGKQRELVARILSGGSTNYADSVKGRFTISRGNAKNTVYLQMNSLKPEDTAVYYCNAVRYLVNYWGQGTQVTVSS

SEQ ID NO: 43 - protein, 121 aa, Artificial Sequence
  VHH sequence 55E07
QVQLQESGGGSVQVGDSLTLSCVASGRSLDIYGMGWFRQAPGKEREFVARITSGGSTYYADSVKGRFTLSRDNAKNTVYLQMNSLKPEDTAVYYCAAGVVVATSPKFYAYWGQGTQVTVSS

SEQ ID NO: 44 - protein, 116 aa, Artificial Sequence
  VHH sequence 55E09
QVQLQESGGGLVQAGGSLRLSCAASKRIFSTYTMGWFRQAPGKEREFVAAIIWSGGRTRYADSVKGRFTISRDNARNTVHLQMNSLEPEDTAVYYCYTRRLGTGYWGQGTQVTVSS

SEQ ID NO: 45 - protein, 115 aa, Artificial Sequence
  VHH sequence 55E10
QVQLQESGGGLVQAGGSLRLSCAASGSTFSIQTIGWYRQAPGKQRDRVATISSGGSTNYADSVKGRFTISRDNAKKTVYLQMNNLKPEDTAVYYCNLRYWFRDYWGQGTQVTVSS

SEQ ID NO: 46 - protein, 115 aa, Artificial Sequence
  VHH sequence 55F04
QVQLQESGGGLVQPGGSLRLSCAASGSTFSINVRGWYRQAPGKQRELVATITSDGSTNYADSVKGRFTISRDNAKNTAYLQMNSLKPEDTAVYYCNAVRLFRQYWGQGTQVTVSS

SEQ ID NO: 47 - protein, 125 aa, Artificial Sequence
  VHH sequence 55F09
QVQLQESGGGLVQPGGSLRLSCAASGSIFRLNAMGWYRQAPGKQRELVAAITPGGGNTTYADSVKGRFTISRDNALNTIYLQMNSLKPEDTAVYYCNAGGSSRWYSSRYYPGGYWGQGTQVTVSS

SEQ ID NO: 48 - protein, 126 aa, Artificial Sequence
  VHH sequence 55F10
QVQLQESGGGLVQAGGSLRLSCATSGGTFSRYAMGWFRQAPGKERELVATIRRSGSSTYYLDSTKGRFTISRDNAKNTVYLQMNSLKLEDTAVYYCAADSSARALVGGPGNRWDYWGQGTQVTVSS

SEQ ID NO: 49 - protein, 118 aa, Artificial Sequence
  VHH sequence 55G02
QVQLQESGGGLVQPGGSLRLSCAASGSIGSINVMGWYRQYPGKQRELVAFITSGGITNYTDSVKGRFAISRDNAQNTVYLQMNSLTPEDTAVYYCHLKNAKNVRPGYWGQGTQVTVSS

SEQ ID NO: 50 - protein, 116 aa, Artificial Sequence
  VHH sequence 55G08
QVQLQESGGGLVQPGGSLRLSCRASGGIFGINAMRWYRQAPGKQRELVASISSGGTTDYVESVKGRFTISRDNATNTVDLQMSALKPEDTAVYYCNFVRFWFPDYWGQGTQVTVSS

SEQ ID NO: 51 - protein, 116 aa, Artificial Sequence
  VHH sequence 56A05
QVQLQESGGGLVQAGGSLRLSCAASGITFMSNTMGWYRQAPGKQRELVASISSGGSTNYADSVKGRFTISRDNAKKTVYLQMNSLKPEDTAVYYCNARRNVFISSWGQGTQVTVSS

SEQ ID NO: 52 - protein, 114 aa, Artificial Sequence
  VHH sequence 56A06
QVQLQESGGGLVQPGGSLRLSCVASGSISVYGMGWYRQAPGKQRELVARITNIGTTNYADSVKGRFTISRDNAKNTVYLQMNSLQPEDTAVYYCNLRRLGRDYWGQGTQVTVSS

SEQ ID NO: 53 - protein, 118 aa, Artificial Sequence
  VHH sequence 56A09
QVQLQESGGGLVQPGGSLRLSCAASRTALRLNSMGWYRQAPGSQRELVATITRGGTTNYADSVKGRFTISREIGNNTVYLQMNSLEPEDTAVYYCNANFGILVGREYWGKGTQVTVSS

SEQ ID NO: 54 - protein, 116 aa, Artificial Sequence
  VHH sequence 56C09
QVQLQESGGGLVQAGGSLRLSCAVSGSIFSILSMAWYRQTPGKQRELVANITSVGSTNYADSVKGRFTISRDIAKKTLYLQMNNLKPEDTAIYYCNTRMPFLGDSWGQGTQVTVSS

SEQ ID NO: 55 - protein, 111 aa, Artificial Sequence
  VHH sequence 56C12
QVQLQESGGGLVQAGGSLRLSCAVSAFSFSNRAVSWYRQAPGKSREWVASISGIRITTYTNSVKGRFIISRDNAKKTVYLQMNDLRPEDTGVYRCYMNRYSGQGTQVTVSS

SEQ ID NO: 56 - protein, 117 aa, Artificial Sequence
  VHH sequence 56D06
QVQLQESGGGSVQPGGSLRLSCAASGTVFFSISAMGWYRQAPGKQRELVAGISRGGSTKYGDFVKGRFTISRDNGKKTIWLQMNNLQPEDTAIYYCRLTSITGTYLWGQGTQVTVSS

SEQ ID NO: 57 - protein, 114 aa, Artificial Sequence
  VHH sequence 56D07
QVQLQESGGGLVQPGGSLRLSCAASGSIFSMKVMGWYRQGPGKLRELVAVITSGGRTNYAESVKGRFTISRDNAKNTVSLQMNSLQPEDTAVYYCYYKTIRPYWGQGTQVTVSS

SEQ ID NO: 58 - protein, 120 aa, Artificial Sequence
  VHH sequence 56D10
QVQLQESGGGLVQAGGSLRLSCAASGITFRITTMGWYRQAPGKQRELVASSSSGGTTNYASSVKGRFTISRDNAKNTVYLQMNSLRPEDTAVYYCNARKFITTPWSTDYWGQGTQVTVSS

SEQ ID NO: 59 - protein, 116 aa, Artificial Sequence
  VHH sequence 56E04
QVQLQESGGGLVQPGDSLRLSCTPSGSIFNHKATGWYRQAPGSQRELVAKITTGGTTNYADSVKGRFTISRDNAKNTVYLQMSSLKPEDTAVYYCNAERYFATTLWGQGTQVTVSS

SEQ ID NO: 60 - protein, 119 aa, Artificial Sequence
  VHH sequence 56E05
QVQLQESGGGLVQAGGSLRLSCAASGITFSNNAGGWYRQAPGQQRELVARISSGGNTNYTDSVKGRFTISRDITKNTLSLQMNNLKPEDSAVYYCNAQRRVILGPRNYWGQGTQVTVSS

SEQ ID NO: 61 - protein, 121 aa, Artificial Sequence
  VHH sequence 56E08
QVQLQESGGGLVQAGGSLRLSCAASGNIFRINDMGWYRQAPGNQRELVATITSANITNYADSVKGRFTISRDNAKNTVYLQMNSLNPEDTAVYYCTAQAKKWRIGPWSDYWGQGTQVTVSS

SEQ ID NO: 62 - protein, 119 aa, Artificial Sequence
  VHH sequence 56F07
QVQLQESGGGLVQPGGSLRLSCAASGRIFSINDMAWYRQAPGKQRELVAIITNDDSTTYADSVKGRFTISRDNAKNTVYLQMNSLKPEDTAVYYCNADINTAIWRRKYWGQGTQVTVSS

SEQ ID NO: 63 - protein, 115 aa, Artificial Sequence
  VHH sequence 56G07
QVQLQESGGGLVQPGGSLRLSCAVSGSRIFIHDMGWHRQAPGEPRELVATITPFGRRNYSEYVKGRFTVSRDIARNTMSLQMSNLKAEDTGMYYCNVRVNGVDYWGQGTQVTVSS

SEQ ID NO: 64 - protein, 119 aa, Artificial Sequence
  VHH sequence 56G08
QVQLQESGGGLVQAGGSLRLSCAISGITFRRPFGISRMGWYRQAPGKERELVATLSRAGTSRYVDSVKGRFTISRDDAKNTLYLQMVSLNPEDTAVYYCYIAQLGTDYWGQGTQVTVSS

SEQ ID NO: 65 - protein, 117 aa, Artificial Sequence
  VHH sequence 56G10
QVQLQESGGGLVQAGGSLRLSCVASGITLRMYQVGWYRQAPGKQRELVAEISSRGTTMYADSVKGRFTISRDGAKNIVYLQMNSLEPEDTAVYYCNARAFAFGRNSWGQGTQVTVSS

SEQ ID NO: 66 - protein, 118 aa, Artificial Sequence
  VHH sequence 56H04
QVQLQESGGGSVQAGGSLRLSCAVSGGTFSNKAMGWYRQSSGKQRALVARISTVGTAHYADSVKGRFTVSKDNAGNTLYLQMNSLKPEDTAVYYCNAQAGRLYLRNYWGQGTQVTVSS

SEQ ID NO: 67 - protein, 119 aa, Artificial Sequence
  VHH sequence 56H05
QVQLQESGGGLVQPGESLRLSCVAAASTSITTFNTMAWYRQAPGKQRELVAQINNRDNTEYADSVKGRFIISRGNAKNTSNLQMNDLKSEDTGIYYCNAKRWSWSTGFWGQGTQVTVSS

SEQ ID NO: 68 - protein, 114 aa, Artificial Sequence
  VHH sequence 56H07
QVQLQESGGGLVQAGGSLRLSCTASGLTFALGTMGWYRQAPGKQRELVASISTGSTNYADSVKGRFTISRDIIKNILYLQMNSLKPEDTAVYSCNARLWWSNYWGQGTQVTVSS

SEQ ID NO: 69 - protein, 118 aa, Artificial Sequence
  VHH sequence 56H08
QVQLQESGGGLVQAGGSLRLSCTASGRTSSVNPMGWYRQAPGKQRELVAVISSDGSTNYADSVKGRFTVSRDNAKNTLYLQMNSLKPEDTAVYYCNANRRWSWGSEYWGQGTQVTVSS

SEQ ID NO: 70 - protein, 119 aa, Artificial Sequence
  VHH sequence 57A06
QVQLQESGGGLVQAGGSLRLSCAASGITFTNNAGGWYRQAPGQQRELVARISSGGNTNYTDSVKGRFTISRDITKNTLSLQMNNLKPEDSAVYYCNAQRRVILGPRNYWGQGTQVTVSS

SEQ ID NO: 71 - protein, 116 aa, Artificial Sequence
  VHH sequence 57B01
QVQLQESGGGLVQAGGSLRLSCEAPVSTFNINAMAWYRQAPGKSRELVARISSGGSTNYADSVKGRFTISRDNAKNTVYLQMNSLKPEDTAVYICYVNRHWGWDYWGQGTQVTVSS

SEQ ID NO: 72 - protein, 122 aa, Artificial Sequence
  VHH sequence 57B07
QVQLQESGGGLVQPGGTLRLSCVASGSFRSINAMGWYRQAPGKQRELVATVDSGGYTNYADSVKGRFTISRDNAKNTVYLQMSSLTPEDTAVYYCYAGIYKWPWSVDARDYWGQGTQVTVSS

SEQ ID NO: 73 - protein, 117 aa, Artificial Sequence
  VHH sequence 57B11
QVQLQESGGGLVQAGGSLRLSCAASGSSISMNSMGWYRQAPGKERERVALIRSSGGTYYADSVKGRFTISRDNAKNTVYLQMNNLKPEDTAVYYCQARRTWLSSESWGQGTQVTVSS

SEQ ID NO: 74 - protein, 122 aa, Artificial Sequence
  VHH sequence 57C07
QVQLQESGGGLVQAGGSLRLSCAVSGSTFGINTMGWYRQAPEKQRELVASISRGGMTNYADSVKGRFIISRDNAKNTVYLQMNSLKPEDTAVYVCNAGIRSRWYGGPITTYWGQGTQVTVSS

SEQ ID NO: 75 - protein, 121 aa, Artificial Sequence
  VHH sequence 57C09
QVQLQESGGGLVQAGGSLRLSCAASGSTGSINAMGWYRQGPGKQRDLVASISSGGATNYADSVKGRFTISRDNSKNTVYLQMSSLKPEDTAVYYCNAKKSRWSWSIVHDYWGQGTQVTVSS

SEQ ID NO: 76 - protein, 118 aa, Artificial Sequence
  VHH sequence 57D02
QVQLQESGGGSVQTGGSLTLSCTTSGSIFGRSDMGWYRQAPGKQRELVATITRRSRTNYAEFVKGRFTISRDSAKNLVTLQMNSLKPEDTNVYYCNARWGAGGIFSTWGQGTQVTVSS

SEQ ID NO: 77 - protein, 120 aa, Artificial Sequence
  VHH sequence 57D09
QVQLQESGGGLVQPGESLRLSCAASGSMSIDAMGWYRQAPGDQRELVASITTGGSTNYADSVKGRFTISRDNAKNTVWLQMNSLKPEDTAVYYCNAKVRLRWFRPPSDYWGQGTQVTVSS

SEQ ID NO: 78 - protein, 122 aa, Artificial Sequence
  VHH sequence 57D10
QVQLQESGGGLVQPGGSLRLSCAASGRLLSISTMGWYRRTPEDQREMVASITKDGTTNYADSVKGRLTISRDNAKNTVYLQMNSLKPDDTAVYVCNARATTWVPYRRDAEFWGQGTQVTVSS

SEQ ID NO: 79 - protein, 120 aa, Artificial Sequence
  VHH sequence 57E07
QVQLQESGGGLVQAGGSLRLSCAASGSIFGINDMGWYRQAPGKQRDLVADITRSGSTHYVDSVKGRFTISRDNAKNTVYLQMNSLKPEDTAVYYCNADSGSHWWNRRDYWGQGTQVTVSS

SEQ ID NO: 80 - protein, 118 aa, Artificial Sequence
  VHH sequence 57E11
QVQLQESGGGLVQPGGSLKLSCAASGFTFSINTMGWYRQAPGKQRELVARISRLRVTNYADSVKGRFTISRDNAKNTVYLQMNSLKPEDTAVYYCNAANWGLAGNEYWGQGTQVTVSS

SEQ ID NO: 81 - protein, 116 aa, Artificial Sequence
  VHH sequence 57G01
QVQLQESGGGLVQAGGSLRPSCTASGSTLLINSMGWYRQAPGKQRELVATISNSGTTNYVDAVKGRFAISRDNANHTVYLQMNSLEPEDTAVYYCNAQTFWRRNYWGQGTQVTVSS

SEQ ID NO: 82 - protein, 116 aa, Artificial Sequence
  VHH sequence 57G07
QVQLQESGGGLVQAGGSLRLSCAVSGSTSRINAMGWYRQAPGKKRESVATIRRGGNTKYADSVKGRFTISRDNANNTVYLQLNSLKPEDTAVYYCNAHSWLDYDYWGRGTQVTVSS

SEQ ID NO: 83 - protein, 114 aa, Artificial Sequence
  VHH sequence 57G08
QVQLQESGGGLVQAGGSLRLSCASRRRINGITMGWYRQAPGKQRELVATIDIHNSTKYADSVKGRFIISRDNGKSMLYLQMNSLKPEDTAVYYCNRIPTFGRYWGQGTQVTVSS

SEQ ID NO: 84 - protein, 118 aa, Artificial Sequence
  VHH sequence 57H08
QVQLQESGGGLVQAGGSLRLSCVASGSTFYTFSTKNVGWYRQAPGKQRELVAQQRYDGSTNYADSLQGRFTISRDNAKRTVYLQMNSLKPEDTAVYICNVNRGFISYWGQGTQVTVSS

SEQ ID NO: 85 - protein, 5 aa, Artificial Sequence
  CDR1 of VHH sequence 41D01
RYGMG

SEQ ID NO: 86 - protein, 5 aa, Artificial Sequence
  CDR1 of VHH sequence 56F11
RNAMG

SEQ ID NO: 87 - protein, 5 aa, Artificial Sequence
  CDR1 of VHH sequence 40F07
SYTMG

SEQ ID NO: 88 - protein, 5 aa, Artificial Sequence
  CDR1 of VHH sequence 41D06
INAMR

SEQ ID NO: 89 - protein, 5 aa, Artificial Sequence
  CDR1 of VHH sequence 41G10
INAMG

SEQ ID NO: 90 - protein, 5 aa, Artificial Sequence
  CDR1 of VHH sequence 41H05
INAMG

SEQ ID NO: 91 - protein, 5 aa, Artificial Sequence
  CDR1 of VHH sequence 42C11
TYVMG

SEQ ID NO: 92 - protein, 5 aa, Artificial Sequence
  CDR1 of VHH sequence 42C12
ISSLG

SEQ ID NO: 93 - protein, 5 aa, Artificial Sequence
  CDR1 of VHH sequence 50D03
TYAMG

SEQ ID NO: 94 - protein, 5 aa, Artificial Sequence
  CDR1 of VHH sequence 50D07
IRDMG

SEQ ID NO: 95 - protein, 5 aa, Artificial Sequence
  CDR1 of VHH sequence 50E02
INAMG

SEQ ID NO: 96 - protein, 5 aa, Artificial Sequence
  CDR1 of VHH sequence 51B08
SYAMG

SEQ ID NO: 97 - protein, 5 aa, Artificial Sequence
  CDR1 of VHH sequence 51C06
SDTMG

SEQ ID NO: 98 - protein, 5 aa, Artificial Sequence
  CDR1 of VHH sequence 51C08
IKTMG

SEQ ID NO: 99 - protein, 5 aa, Artificial Sequence
  CDR1 of VHH sequence 52A01
LGTMG

SEQ ID NO: 100 - protein, 5 aa, Artificial Sequence
  CDR1 of VHH sequence 52B01
INVMG

SEQ ID NO: 101 - protein, 5 aa, Artificial Sequence
  CDR1 of VHH sequence 52G05
ISAMG

SEQ ID NO: 102 - protein, 5 aa, Artificial Sequence
  CDR1 of VHH sequence 53A01
INTMG

SEQ ID NO: 103 - protein, 5 aa, Artificial Sequence
  CDR1 of VHH sequence 53F05
LNAMG

SEQ ID NO: 104 - protein, 5 aa, Artificial Sequence
  CDR1 of VHH sequence 54A02
RYGMG

SEQ ID NO: 105 - protein, 5 aa, Artificial Sequence
  CDR1 of VHH sequence 54B01
RYTMG

SEQ ID NO: 106 - protein, 5 aa, Artificial Sequence
  CDR1 of VHH sequence 54C01
RYAMG

SEQ ID NO: 107 - protein, 5 aa, Artificial Sequence
  CDR1 of VHH sequence 54C04
INAMG

SEQ ID NO: 108 - protein, 5 aa, Artificial Sequence
  CDR1 of VHH sequence 54C08
INAMG

SEQ ID NO: 109 - protein, 5 aa, Artificial Sequence
  CDR1 of VHH sequence 54C10
VNDMG

SEQ ID NO: 110 - protein, 5 aa, Artificial Sequence
  CDR1 of VHH sequence 54C11
VNDMA

SEQ ID NO: 111 - protein, 5 aa, Artificial Sequence
  CDR1 of VHH sequence 54D03
INAMR

SEQ ID NO: 112 - protein, 5 aa, Artificial Sequence
  CDR1 of VHH sequence 54D06
INAMG

SEQ ID NO: 113 - protein, 5 aa, Artificial Sequence
  CDR1 of VHH sequence 54D10
IHAMG

SEQ ID NO: 114 - protein, 5 aa, Artificial Sequence
  CDR1 of VHH sequence 54E01
INPMG

SEQ ID NO: 115 - protein, 5 aa, Artificial Sequence
  CDR1 of VHH sequence 54E05
INTMG

SEQ ID NO: 116 - protein, 5 aa, Artificial Sequence
  CDR1 of VHH sequence 54E10
FNAMG

SEQ ID NO: 117 - protein, 5 aa, Artificial Sequence
  CDR1 of VHH sequence 54F01
LNLMG

SEQ ID NO: 118 - protein, 5 aa, Artificial Sequence
  CDR1 of VHH sequence 54F02
INTMG

SEQ ID NO: 119 - protein, 5 aa, Artificial Sequence
  CDR1 of VHH sequence 54G01
VNAMG

SEQ ID NO: 120 - protein, 5 aa, Artificial Sequence
  CDR1 of VHH sequence 54G08
FNLMG

SEQ ID NO: 121 - protein, 5 aa, Artificial Sequence
  CDR1 of VHH sequence 54G09
IRDMG

SEQ ID NO: 122 - protein, 5 aa, Artificial Sequence
  CDR1 of VHH sequence 55B02
INSMN

SEQ ID NO: 123 - protein, 5 aa, Artificial Sequence
  CDR1 of VHH sequence 55B05
GYTVA

SEQ ID NO: 124 - protein, 5 aa, Artificial Sequence
  CDR1 of VHH sequence 55C05
MKAMG

SEQ ID NO: 125 - protein, 5 aa, Artificial Sequence
  CDR1 of VHH sequence 55D08
ISAMG

SEQ ID NO: 126 - protein, 5 aa, Artificial Sequence
  CDR1 of VHH sequence 55E02
SNAMA

SEQ ID NO: 127 - protein, 5 aa, Artificial Sequence
  CDR1 of VHH sequence 55E07
IYGMG

SEQ ID NO: 128 - protein, 5 aa, Artificial Sequence
  CDR1 of VHH sequence 55E09
TYTMG

SEQ ID NO: 129 - protein, 5 aa, Artificial Sequence
  CDR1 of VHH sequence 55E10
IQTIG

SEQ ID NO: 130 - protein, 5 aa, Artificial Sequence
  CDR1 of VHH sequence 55F04
INVRG

SEQ ID NO: 131 - protein, 5 aa, Artificial Sequence
  CDR1 of VHH sequence 55F09
LNAMG

SEQ ID NO: 132 - protein, 5 aa, Artificial Sequence
  CDR1 of VHH sequence 55F10
RYAMG

SEQ ID NO: 133 - protein, 5 aa, Artificial Sequence
  CDR1 of VHH sequence 55G02
INVMG

SEQ ID NO: 134 - protein, 5 aa, Artificial Sequence
  CDR1 of VHH sequence 55G08
INAMR

SEQ ID NO: 135 - protein, 5 aa, Artificial Sequence
  CDR1 of VHH sequence 56A05
SNTMG

SEQ ID NO: 136 - protein, 5 aa, Artificial Sequence
  CDR1 of VHH sequence 56A06
VYGMG

SEQ ID NO: 137 - protein, 5 aa, Artificial Sequence
  CDR1 of VHH sequence 56A09
LNSMG

SEQ ID NO: 138 - protein, 5 aa, Artificial Sequence
  CDR1 of VHH sequence 56C09
ILSMA

SEQ ID NO: 139 - protein, 5 aa, Artificial Sequence
  CDR1 of VHH sequence 56C12
NRAVS

SEQ ID NO: 140 - protein, 5 aa, Artificial Sequence
  CDR1 of VHH sequence 56D06
ISAMG

SEQ ID NO: 141 - protein, 5 aa, Artificial Sequence
  CDR1 of VHH sequence 56D07
MKVMG

SEQ ID NO: 142 - protein, 5 aa, Artificial Sequence
  CDR1 of VHH sequence 56D10
ITTMG

SEQ ID NO: 143 - protein, 5 aa, Artificial Sequence
  CDR1 of VHH sequence 56E04
HKATG

SEQ ID NO: 144 - protein, 5 aa, Artificial Sequence
  CDR1 of VHH sequence 56E05
NNAGG

SEQ ID NO: 145 - protein, 5 aa, Artificial Sequence
  CDR1 of VHH sequence 56E08
INDMG

SEQ ID NO: 146 - protein, 5 aa, Artificial Sequence
  CDR1 of VHH sequence 56F07
INDMA

SEQ ID NO: 147 - protein, 5 aa, Artificial Sequence
  CDR1 of VHH sequence 56G07
IHDMG

SEQ ID NO: 148 - protein, 5 aa, Artificial Sequence
  CDR1 of VHH sequence 56G08
ISRMG

SEQ ID NO: 149 - protein, 5 aa, Artificial Sequence
  CDR1 of VHH sequence 56G10
MYQVG

SEQ ID NO: 150 - protein, 5 aa, Artificial Sequence
  CDR1 of VHH sequence 56H04
NKAMG

SEQ ID NO: 151 - protein, 5 aa, Artificial Sequence
  CDR1 of VHH sequence 56H05
FNTMA

SEQ ID NO: 152 - protein, 5 aa, Artificial Sequence
  CDR1 of VHH sequence 56H07
LGTMG

SEQ ID NO: 153 - protein, 5 aa, Artificial Sequence
  CDR1 of VHH sequence 56H08
VNPMG

SEQ ID NO: 154 - protein, 5 aa, Artificial Sequence
  CDR1 of VHH sequence 57A06
NNAGG

SEQ ID NO: 155 - protein, 5 aa, Artificial Sequence
  CDR1 of VHH sequence 57B01
INAMA

SEQ ID NO: 156 - protein, 5 aa, Artificial Sequence
  CDR1 of VHH sequence 57B07
INAMG

SEQ ID NO: 157 - protein, 5 aa, Artificial Sequence
  CDR1 of VHH sequence 57B11
MNSMG

SEQ ID NO: 158 - protein, 5 aa, Artificial Sequence
  CDR1 of VHH sequence 57C07
INTMG

SEQ ID NO: 159 - protein, 5 aa, Artificial Sequence
  CDR1 of VHH sequence 57C09
INAMG

SEQ ID NO: 160 - protein, 5 aa, Artificial Sequence
  CDR1 of VHH sequence 57D02
RSDMG

SEQ ID NO: 161 - protein, 5 aa, Artificial Sequence
  CDR1 of VHH sequence 57D09
IDAMG

SEQ ID NO: 162 - protein, 5 aa, Artificial Sequence
  CDR1 of VHH sequence 57D10
ISTMG

SEQ ID NO: 163 - protein, 5 aa, Artificial Sequence
  CDR1 of VHH sequence 57E07
INDMG

SEQ ID NO: 164 - protein, 5 aa, Artificial Sequence
  CDR1 of VHH sequence 57E11
INTMG

SEQ ID NO: 165 - protein, 5 aa, Artificial Sequence
  CDR1 of VHH sequence 57G01
INSMG

SEQ ID NO: 166 - protein, 5 aa, Artificial Sequence
  CDR1 of VHH sequence 57G07
INAMG

SEQ ID NO: 167 - protein, 5 aa, Artificial Sequence
  CDR1 of VHH sequence 57G08
GITMG

SEQ ID NO: 168 - protein, 5 aa, Artificial Sequence
  CDR1 of VHH sequence 57H08
TKNVG

SEQ ID NO: 169 - protein, 16 aa, Artificial Sequence
  CDR2 of VHH sequence 41D01
SITRGGTTTYADSVKG

SEQ ID NO: 170 - protein, 16 aa, Artificial Sequence
  CDR2 of VHH sequence 56F11
TITSGGTTNYADSVKG

SEQ ID NO: 171 - protein, 16 aa, Artificial Sequence
  CDR2 of VHH sequence 40F07
SIEGGGNTDYADSVKG

SEQ ID NO: 172 - protein, 16 aa, Artificial Sequence
  CDR2 of VHH sequence 41D06
SISSGGNTNYSESVKG

SEQ ID NO: 173 - protein, 16 aa, Artificial Sequence
  CDR2 of VHH sequence 41G10
TITSGGTTNYADSVKG

SEQ ID NO: 174 - protein, 16 aa, Artificial Sequence
  CDR2 of VHH sequence 41H05
TITSGANTNYTDSVKG

SEQ ID NO: 175 - protein, 16 aa, Artificial Sequence
  CDR2 of VHH sequence 42C11
TITSSGKTNYAASVKG

SEQ ID NO: 176 - protein, 16 aa, Artificial Sequence
  CDR2 of VHH sequence 42C12
SATSGGDTTYADSVKG

SEQ ID NO: 177 - protein, 16 aa, Artificial Sequence
  CDR2 of VHH sequence 50D03
TITSSGKTNYAASVKG

SEQ ID NO: 178 - protein, 16 aa, Artificial Sequence
  CDR2 of VHH sequence 50D07
TITSDQSTNYADSVKG

SEQ ID NO: 179 - protein, 16 aa, Artificial Sequence
  CDR2 of VHH sequence 50E02
AITSDGSTNYADSVKG

SEQ ID NO: 180 - protein, 16 aa, Artificial Sequence
  CDR2 of VHH sequence 51B08
GISSGGSTKYADSVRG

SEQ ID NO: 181 - protein, 16 aa, Artificial Sequence
  CDR2 of VHH sequence 51C06
AITTGGNTNYADSVKG

SEQ ID NO: 182 - protein, 16 aa, Artificial Sequence
  CDR2 of VHH sequence 51C08
TISNGGSTNYADSVKG

SEQ ID NO: 183 - protein, 15 aa, Artificial Sequence
  CDR2 of VHH sequence 52A01
SISTGSTNYADSVKG

SEQ ID NO: 184 - protein, 16 aa, Artificial Sequence
  CDR2 of VHH sequence 52B01
TISRGGSTNYADSVKG

SEQ ID NO: 185 - protein, 16 aa, Artificial Sequence
  CDR2 of VHH sequence 52G05
SITRRGSTNYADSVKD

SEQ ID NO: 186 - protein, 16 aa, Artificial Sequence
  CDR2 of VHH sequence 53A01
SISSGGWTNYADSVKG

SEQ ID NO: 187 - protein, 16 aa, Artificial Sequence
  CDR2 of VHH sequence 53F05
SITTGGSTNYAEPVKG

SEQ ID NO: 188 - protein, 17 aa, Artificial Sequence
  CDR2 of VHH sequence 54A02
ANRWSGGSTYYADSVRG

SEQ ID NO: 189 - protein, 17 aa, Artificial Sequence
  CDR2 of VHH sequence 54B01
GITWTGGSTDYADSVKG

SEQ ID NO: 190 - protein, 17 aa, Artificial Sequence
  CDR2 of VHH sequence 54C01
AISWSGGSTYYADSVKD

SEQ ID NO: 191 - protein, 16 aa, Artificial Sequence
  CDR2 of VHH sequence 54C04
DMTSGGSINYADSVSG

SEQ ID NO: 192 - protein, 16 aa, Artificial Sequence
  CDR2 of VHH sequence 54C08
SITSGGSTNYADSVKG

SEQ ID NO: 193 - protein, 16 aa, Artificial Sequence
  CDR2 of VHH sequence 54C10
QITRRGSTNYADSVKG

SEQ ID NO: 194 - protein, 16 aa, Artificial Sequence
  CDR2 of VHH sequence 54C11
NITRGGSTNYADSVKG

SEQ ID NO: 195 - protein, 16 aa, Artificial Sequence
  CDR2 of VHH sequence 54D03
SISSGGNTNYSESVKG

SEQ ID NO: 196 - protein, 16 aa, Artificial Sequence
  CDR2 of VHH sequence 54D06
TITRGGITNYADSVKG

SEQ ID NO: 197 - protein, 16 aa, Artificial Sequence
  CDR2 of VHH sequence 54D10
ITSTSGTTDYTDSVKG

SEQ ID NO: 198 - protein, 16 aa, Artificial Sequence
  CDR2 of VHH sequence 54E01
AITSGGSTNYADYVKG

SEQ ID NO: 199 - protein, 16 aa, Artificial Sequence
  CDR2 of VHH sequence 54E05
AITNRGSTNYADFVKG

SEQ ID NO: 200 - protein, 16 aa, Artificial Sequence
  CDR2 of VHH sequence 54E10
AITRGGSTNYADSVKG

SEQ ID NO: 201 - protein, 16 aa, Artificial Sequence
  CDR2 of VHH sequence 54F01
TITRGGSTNYADSVKG

SEQ ID NO: 202 - protein, 16 aa, Artificial Sequence
  CDR2 of VHH sequence 54F02
TITSGGTTNYADSVKN

SEQ ID NO: 203 - protein, 16 aa, Artificial Sequence
  CDR2 of VHH sequence 54G01
IISSNSTSNYADSVKG

SEQ ID NO: 204 - protein, 16 aa, Artificial Sequence
  CDR2 of VHH sequence 54G08
AITSSSNTNYADSVKG

SEQ ID NO: 205 - protein, 16 aa, Artificial Sequence
  CDR2 of VHH sequence 54G09
TITSDQSTNYADSVKG

SEQ ID NO: 206 - protein, 16 aa, Artificial Sequence
  CDR2 of VHH sequence 55B02
DMRSDGSTNYADSVKG

SEQ ID NO: 207 - protein, 17 aa, Artificial Sequence
  CDR2 of VHH sequence 55B05
RISWSGIMAYYAESVKG

SEQ ID NO: 208 - protein, 16 aa, Artificial Sequence
  CDR2 of VHH sequence 55C05
QITRGDSTNYADSVKG

SEQ ID NO: 209 - protein, 16 aa, Artificial Sequence
  CDR2 of VHH sequence 55D08
TITSAGSSNYSDSVKG

SEQ ID NO: 210 - protein, 16 aa, Artificial Sequence
  CDR2 of VHH sequence 55E02
RILSGGSTNYADSVKG

SEQ ID NO: 211 - protein, 16 aa, Artificial Sequence
  CDR2 of VHH sequence 55E07
RITSGGSTYYADSVKG

SEQ ID NO: 212 - protein, 17 aa, Artificial Sequence
  CDR2 of VHH sequence 55E09
AIIWSGGRTRYADSVKG

SEQ ID NO: 213 - protein, 16 aa, Artificial Sequence
  CDR2 of VHH sequence 55E10
TISSGGSTNYADSVKG

SEQ ID NO: 214 - protein, 16 aa, Artificial Sequence
  CDR2 of VHH sequence 55F04
TITSDGSTNYADSVKG

SEQ ID NO: 215 - protein, 17 aa, Artificial Sequence
  CDR2 of VHH sequence 55F09
AITPGGGNTTYADSVKG

SEQ ID NO: 216 - protein, 17 aa, Artificial Sequence
  CDR2 of VHH sequence 55F10
TIRRSGSSTYYLDSTKG

SEQ ID NO: 217 - protein, 16 aa, Artificial Sequence
  CDR2 of VHH sequence 55G02
FITSGGITNYTDSVKG

SEQ ID NO: 218 - protein, 16 aa, Artificial Sequence
  CDR2 of VHH sequence 55G08
SISSGGTTDYVESVKG

SEQ ID NO: 219 - protein, 16 aa, Artificial Sequence
  CDR2 of VHH sequence 56A05
SISSGGSTNYADSVKG

SEQ ID NO: 220 - protein, 16 aa, Artificial Sequence
  CDR2 of VHH sequence 56A06
RITNIGTTNYADSVKG

SEQ ID NO: 221 - protein, 16 aa, Artificial Sequence
  CDR2 of VHH sequence 56A09
TITRGGTTNYADSVKG

SEQ ID NO: 222 - protein, 16 aa, Artificial Sequence
  CDR2 of VHH sequence 56C09
NITSVGSTNYADSVKG

SEQ ID NO: 223 - protein, 16 aa, Artificial Sequence
  CDR2 of VHH sequence 56C12
SISGIRITTYTNSVKG

SEQ ID NO: 224 - protein, 16 aa, Artificial Sequence
  CDR2 of VHH sequence 56D07
VITSGGRTNYAESVKG

SEQ ID NO: 225 - protein, 16 aa, Artificial Sequence
  CDR2 of VHH sequence 56D07
VITSGGRTNYAESVKG

SEQ ID NO: 226 - protein, 16 aa, Artificial Sequence
  CDR2 of VHH sequence 56D10
SSSSGGTTNYASSVKG

SEQ ID NO: 227 - protein, 16 aa, Artificial Sequence
  CDR2 of VHH sequence 56E04
KITTGGTTNYADSVKG

SEQ ID NO: 228 - protein, 16 aa, Artificial Sequence
  CDR2 of VHH sequence 56E05
RISSGGNTNYTDSVKG

SEQ ID NO: 229 - protein, 16 aa, Artificial Sequence
  CDR2 of VHH sequence 56E08
TITSANITNYADSVKG

SEQ ID NO: 230 - protein, 16 aa, Artificial Sequence
  CDR2 of VHH sequence 56F07
IITNDDSTTYADSVKG

SEQ ID NO: 231 - protein, 16 aa, Artificial Sequence
  CDR2 of VHH sequence 56G07
TITPFGRRNYSEYVKG

SEQ ID NO: 232 - protein, 16 aa, Artificial Sequence
  CDR2 of VHH sequence 56G08
TLSRAGTSRYVDSVKG

SEQ ID NO: 233 - protein, 16 aa, Artificial Sequence
  CDR2 of VHH sequence 56G10
EISSRGTTMYADSVKG

SEQ ID NO: 234 - protein, 16 aa, Artificial Sequence
  CDR2 of VHH sequence 56H04
RISTVGTAHYADSVKG

SEQ ID NO: 235 - protein, 16 aa, Artificial Sequence
  CDR2 of VHH sequence 56H05
QINNRDNTEYADSVKG

SEQ ID NO: 236 - protein, 15 aa, Artificial Sequence
  CDR2 of VHH sequence 56H07
SISTGSTNYADSVKG

SEQ ID NO: 237 - protein, 16 aa, Artificial Sequence
  CDR2 of VHH sequence 56H08
VISSDGSTNYADSVKG

SEQ ID NO: 238 - protein, 16 aa, Artificial Sequence
  CDR2 of VHH sequence 57A06
RISSGGNTNYTDSVKG

SEQ ID NO: 239 - protein, 16 aa, Artificial Sequence
  CDR2 of VHH sequence 57B01
RISSGGSTNYADSVKG

SEQ ID NO: 240 - protein, 16 aa, Artificial Sequence
  CDR2 of VHH sequence 57B07
TVDSGGYTNYADSVKG

SEQ ID NO: 241 - protein, 16 aa, Artificial Sequence
  CDR2 of VHH sequence 57B11
LIRSSGGTYYADSVKG

SEQ ID NO: 242 - protein, 16 aa, Artificial Sequence
  CDR2 of VHH sequence 57C07
SISRGGMTNYADSVKG

SEQ ID NO: 243 - protein, 16 aa, Artificial Sequence
  CDR2 of VHH sequence 57C09
SISSGGATNYADSVKG

SEQ ID NO: 244 - protein, 16 aa, Artificial Sequence
  CDR2 of VHH sequence 57D02
TITRRSRTNYAEFVKG

SEQ ID NO: 245 - protein, 16 aa, Artificial Sequence
  CDR2 of VHH sequence 57D09
SITTGGSTNYADSVKG

SEQ ID NO: 246 - protein, 16 aa, Artificial Sequence
  CDR2 of VHH sequence 57D10
SITKDGTTNYADSVKG

SEQ ID NO: 247 - protein, 16 aa, Artificial Sequence
  CDR2 of VHH sequence 57E07
DITRSGSTHYVDSVKG

SEQ ID NO: 248 - protein, 16 aa, Artificial Sequence
  CDR2 of VHH sequence 57E11
RISRLRVTNYADSVKG

SEQ ID NO: 249 - protein, 16 aa, Artificial Sequence
  CDR2 of VHH sequence 57G01
TISNSGTTNYVDAVKG

SEQ ID NO: 250 - protein, 16 aa, Artificial Sequence
  CDR2 of VHH sequence 57G07
TIRRGGNTKYADSVKG

SEQ ID NO: 251 - protein, 16 aa, Artificial Sequence
  CDR2 of VHH sequence 57G08
TIDIHNSTKYADSVKG

SEQ ID NO: 252 - protein, 16 aa, Artificial Sequence
  CDR2 of VHH sequence 57H08
QQRYDGSTNYADSLQG

SEQ ID NO: 253 - protein, 7 aa, Artificial Sequence
  CDR3 of VHH sequence 41D01
RSIWRDY

SEQ ID NO: 254 - protein, 11 aa, Artificial Sequence
  CDR3 of VHH sequence 56F07
DINTAIWRRKY

SEQ ID NO: 255 - protein, 10 aa, Artificial Sequence
  CDR3 of VHH sequence 40F07
ARTWSIFRNY

SEQ ID NO: 256 - protein, 8 aa, Artificial Sequence
  CDR3 of VHH sequence 41D06
VRLWFPDY

SEQ ID NO: 257 - protein, 13 aa, Artificial Sequence
  CDR3 of VHH sequence 41G10
EARRYFTRASQVY

SEQ ID NO: 258 - protein, 12 aa, Artificial Sequence
  CDR3 of VHH sequence 41H05
VGRRWYGGYVEL

SEQ ID NO: 259 - protein, 11 aa, Artificial Sequence
  CDR3 of VHH sequence 42C11
DRWVLTRWSNY

SEQ ID NO: 260 - protein, 11 aa, Artificial Sequence
  CDR3 of VHH sequence 42C12
QRGVAWTRKEY

SEQ ID NO: 261 - protein, 11 aa, Artificial Sequence
  CDR3 of VHH sequence 50D03
DRWVLTRWSNY

SEQ ID NO: 262 - protein, 12 aa, Artificial Sequence
  CDR3 of VHH sequence 50D07
RVRTVLRGWRDY

SEQ ID NO: 263 - protein, 11 aa, Artificial Sequence
  CDR3 of VHH sequence 50E02
RRRTFLKSSDY

SEQ ID NO: 264 - protein, 15 aa, Artificial Sequence
  CDR3 of VHH sequence 51B08
KYGRWTYTGRPEYDS

SEQ ID NO: 265 - protein, 8 aa, Artificial Sequence
  CDR3 of VHH sequence 51C06
RRRWSRDF

SEQ ID NO: 266 - protein, 11 aa, Artificial Sequence
  CDR3 of VHH sequence 51C08
RQQFIGAPYEY

SEQ ID NO: 267 - protein, 7 aa, Artificial Sequence
  CDR3 of VHH sequence 52A01
RLLWSNY

SEQ ID NO: 268 - protein, 9 aa, Artificial Sequence
  CDR3 of VHH sequence 52B01
AGWVGVTNY

SEQ ID NO: 269 - protein, 9 aa, Artificial Sequence
  CDR3 of VHH sequence 52G05
RRYYTRNDY

SEQ ID NO: 270 - protein, 6 aa, Artificial Sequence
  CDR3 of VHH sequence 53A01
GAIGNW

SEQ ID NO: 271 - protein, 9 aa, Artificial Sequence
  CDR3 of VHH sequence 53F05
ERRWGLPNY

SEQ ID NO: 272 - protein, 17 aa, Artificial Sequence
  CDR3 of VHH sequence 54A02
YAHITAWGMRNDYEYDY

SEQ ID NO: 273 - protein, 16 aa, Artificial Sequence
  CDR3 of VHH sequence 54B01
GNLLRLAGQLRRGYDS

SEQ ID NO: 274 - protein, 20 aa, Artificial Sequence
  CDR3 of VHH sequence 54C01
RNRAGPHYSRGYTAGQEYDY

SEQ ID NO: 275 - protein, 13 aa, Artificial Sequence
  CDR3 of VHH sequence 54C04
NLRTAFWRNGNDY

SEQ ID NO: 276 - protein, 7 aa, Artificial Sequence
  CDR3 of VHH sequence 54C08
GPWYRRS

SEQ ID NO: 277 - protein, 8 aa, Artificial Sequence
  CDR3 of VHH sequence 54C10
DLAVRGRY

SEQ ID NO: 278 - protein, 11 aa, Artificial Sequence
  CDR3 of VHH sequence 54C11
RIGFGWTAKAY

SEQ ID NO: 279 - protein, 8 aa, Artificial Sequence
  CDR3 of VHH sequence 54D03
VRLWFPDY

SEQ ID NO: 280 - protein, 8 aa, Artificial Sequence
  CDR3 of VHH sequence 54D06
RSWVGPEY

SEQ ID NO: 281 - protein, 12 aa, Artificial Sequence
  CDR3 of VHH sequence 54D10
KTRTWYNGKYDY

SEQ ID NO: 282 - protein, 9 aa, Artificial Sequence
  CDR3 of VHH sequence 54E01
RSTLWRRDY

SEQ ID NO: 283 - protein, 9 aa, Artificial Sequence
  CDR3 of VHH sequence 54E05
HRSWPRYDS

SEQ ID NO: 284 - protein, 10 aa, Artificial Sequence
  CDR3 of VHH sequence 54E10
ESRIFRRYDY

SEQ ID NO: 285 - protein, 7 aa, Artificial Sequence
  CDR3 of VHH sequence 54F01
DRGWSSY

SEQ ID NO: 286 - protein, 11 aa, Artificial Sequence
  CDR3 of VHH sequence 54F02
HQRAWARSYVY

SEQ ID NO: 287 - protein, 9 aa, Artificial Sequence
  CDR3 of VHH sequence 54G01
KRSWFSQEY

SEQ ID NO: 288 - protein, 13 aa, Artificial Sequence
  CDR3 of VHH sequence 54G08
QYTITPWGIKKDY

SEQ ID NO: 289 - protein, 12 aa, Artificial Sequence
  CDR3 of VHH sequence 54G09
RVRTVLRGWRDY

SEQ ID NO: 290 - protein, 9 aa, Artificial Sequence
  CDR3 of VHH sequence 55B02
NSIFRSRDY

SEQ ID NO: 291 - protein, 16 aa, Artificial Sequence
  CDR3 of VHH sequence 55B05
RSQIRSPWSSLDDYDR

SEQ ID NO: 292 - protein, 8 aa, Artificial Sequence
  CDR3 of VHH sequence 55C05
DRFFGRDY

SEQ ID NO: 293 - protein, 12 aa, Artificial Sequence
  CDR3 of VHH sequence 55D08
VYSRPLLGPLEV

SEQ ID NO: 294 - protein, 7 aa, Artificial Sequence
  CDR3 of VHH sequence 55E02
VRYLVNY

SEQ ID NO: 295 - protein, 13 aa, Artificial Sequence
  CDR3 of VHH sequence 55E07
GVVVATSPKFYAY

SEQ ID NO: 296 - protein, 7 aa, Artificial Sequence
  CDR3 of VHH sequence 55E09
RRLGTGY

SEQ ID NO: 297 - protein, 7 aa, Artificial Sequence
  CDR3 of VHH sequence 55E10
RYWFRDY

SEQ ID NO: 298 - protein, 7 aa, Artificial Sequence
  CDR3 of VHH sequence 55F04
VRLFRQY

SEQ ID NO: 299 - protein, 16 aa, Artificial Sequence
  CDR3 of VHH sequence 55F09
GGSSRWYSSRYYPGGY

SEQ ID NO: 300 - protein, 17 aa, Artificial Sequence
  CDR3 of VHH sequence 55F10
DSSARALVGGPGNRWDY

SEQ ID NO: 301 - protein, 10 aa, Artificial Sequence
  CDR3 of VHH sequence 55G02
KNAKNVRPGY

SEQ ID NO: 302 - protein, 8 aa, Artificial Sequence
  CDR3 of VHH sequence 55G08
VRFWFPDY

SEQ ID NO: 303 - protein, 8 aa, Artificial Sequence
  CDR3 of VHH sequence 56A05
RRNVFISS

SEQ ID NO: 304 - protein, 7 aa, Artificial Sequence
  CDR3 of VHH sequence 56A06
RRLGRDY

SEQ ID NO: 305 - protein, 10 aa, Artificial Sequence
  CDR3 of VHH sequence 56A09
NFGILVGREY

SEQ ID NO: 306 - protein, 8 aa, Artificial Sequence
  CDR3 of VHH sequence 56C09
RMPFLGDS

SEQ ID NO: 307 - protein, 8 aa, Artificial Sequence
  CDR3 of VHH sequence 56D06
TSITGTYL

SEQ ID NO: 308 - protein, 6 aa, Artificial Sequence
  CDR3 of VHH sequence 56D07
KTIRPY

SEQ ID NO: 309 - protein, 12 aa, Artificial Sequence
  CDR3 of VHH sequence 56D10
RKFITTPWSTDY

SEQ ID NO: 310 - protein, 8 aa, Artificial Sequence
  CDR3 of VHH sequence 56E04
ERYFATTL

SEQ ID NO: 311 - protein, 11 aa, Artificial Sequence
  CDR3 of VHH sequence 56E05
QRRVILGPRNY

SEQ ID NO: 312 - protein, 13 aa, Artificial Sequence
  CDR3 of VHH sequence 56E08
QAKKWRIGPWSDY

SEQ ID NO: 313 - protein, 13 aa, Artificial Sequence
  CDR3 of VHH sequence 56F11
NTRRIFGGTVREY

SEQ ID NO: 314 - protein, 7 aa, Artificial Sequence
  CDR3 of VHH sequence 56G07
RVNGVDY

SEQ ID NO: 315 - protein, 7 aa, Artificial Sequence
  CDR3 of VHH sequence 56G08
AQLGTDY

SEQ ID NO: 316 - protein, 9 aa, Artificial Sequence
  CDR3 of VHH sequence 56G10
RAFAFGRNS

SEQ ID NO: 317 - protein, 10 aa, Artificial Sequence
  CDR3 of VHH sequence 56H04
QAGRLYLRNY

SEQ ID NO: 318 - protein, 9 aa, Artificial Sequence
  CDR3 of VHH sequence 56H05
KRWSWSTGF

SEQ ID NO: 319 - protein, 7 aa, Artificial Sequence
  CDR3 of VHH sequence 56H07
RLWWSNY

SEQ ID NO: 320 - protein, 10 aa, Artificial Sequence
  CDR3 of VHH sequence 56H08
NRRWSWGSEY

SEQ ID NO: 321 - protein, 11 aa, Artificial Sequence
  CDR3 of VHH sequence 57A06
QRRVILGPRNY

SEQ ID NO: 322 - protein, 8 aa, Artificial Sequence
  CDR3 of VHH sequence 57B01
NRHWGWDY

SEQ ID NO: 323 - protein, 14 aa, Artificial Sequence
  CDR3 of VHH sequence 57B07
GIYKWPWSVDARDY

SEQ ID NO: 324 - protein, 9 aa, Artificial Sequence
  CDR3 of VHH sequence 57B11
RRTWLSSES

SEQ ID NO: 325 - protein, 14 aa, Artificial Sequence
  CDR3 of VHH sequence 57C07
GIRSRWYGGPITTY

SEQ ID NO: 326 - protein, 13 aa, Artificial Sequence
  CDR3 of VHH sequence 57C09
KKSRWSWSIVHDY

SEQ ID NO: 327 - protein, 10 aa, Artificial Sequence
  CDR3 of VHH sequence 57D02
RWGAGGIFST

SEQ ID NO: 328 - protein, 13 aa, Artificial Sequence
  CDR3 of VHH sequence 57D09
KVRLRWFRPPSDY

SEQ ID NO: 329 - protein, 14 aa, Artificial Sequence
  CDR3 of VHH sequence 57D10
RATTWVPYRRDAEF

SEQ ID NO: 330 - protein, 12 aa, Artificial Sequence
  CDR3 of VHH sequence 57E07
DSGSHWWNRRDY

SEQ ID NO: 331 - protein, 10 aa, Artificial Sequence
  CDR3 of VHH sequence 57E11
ANWGLAGNEY

SEQ ID NO: 332 - protein, 8 aa, Artificial Sequence
  CDR3 of VHH sequence 57G01
QTFWRRNY

SEQ ID NO: 333 - protein, 8 aa, Artificial Sequence
  CDR3 of VHH sequence 57G07
HSWLDYDY

SEQ ID NO: 334 - protein, 7 aa, Artificial Sequence
  CDR3 of VHH sequence 57G08
IPTFGRY

SEQ ID NO: 335 - protein, 7 aa, Artificial Sequence
  CDR3 of VHH sequence 57H08
NRGFISY

SEQ ID NO: 336 - DNA, 345 bp, Artificial Sequence
  VHH 41D01
caggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttca

SEQ ID NO: 337 - DNA, 363 bp, Artificial Sequence
  VHH 56F11
caggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttca

SEQ ID NO: 338 - DNA, 456 bp, Artificial Sequence
  41D01_His_KDEL
atggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcatggtgctgctaaggatgagctt

SEQ ID NO: 339 - DNA, 1131 bp, Artificial Sequence
  sec_41D01_hinge_Fc_His
atggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcacctagaatccctaagccttctacccctcctggatcttcttgtcctcctggaaatatcctcggtggaccttctgtgttcatcttcccacctaagcctaaggatgctctcatgatctcactcacccctaaggttacatgcgttgtggtggatgtgtctgaggatgatcctgatgtgcacgtgtcatggttcgtggataacaaagaggtgcacactgcttggactcagcctagagaagctcagtacaactctaccttcagggtggtgtctgctctccctatccaacaccaagattggatgaggggtaaagagttcaagtgcaaggtgaacaacaaggctctccctgctcctatcgagaggactatctctaaacctaagggaagggctcagacccctcaagtgtatacaattcctccacctagggaacagatgtctaagaagaaggtttcactcacttgcctcgtgaccaacttcttcagtgaggctatctctgttgagtgggagaggaatggtgagcttgagcaggattacaagaacacccctcctatcctcgattctgatggaacctacttcctctactctaagctcaccgtggataccgattcttggttgcagggtgagatcttcacttgctctgttgtgcatgaggctctccacaaccatcacacccagaagaacctcagtagatctcctggtaaagcggccgcacatcatcatcaccatcat

SEQ ID NO: 340 - DNA, 807 bp, Artificial Sequence
  sec_41D01-9GS-41D01_His
atggcaaacaagcttttcttggtgtgcgctaccttcgctctttgcttccttttgactaacgctcaggttcaattgcaagagtctggtggtggacttgttcaggctggtggatctcttagactttcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcaggtggtggtggaagtggtggtggttctcaagttcaactgcaggaatcaggtggtggattggtgcaagctggtggttctttgaggttgtcatgtgctgctagtggtaggaccttcagtaggtacggtatgggatggtttagacagttgcctggtaagcagagggaactcgtgacttcaatcactagaggtggaaccactacttacgcagatagtgttaagggaagattcactatcagtagagataatgcaaagaacactgtttacttgcaaatgaactcattgaagccagaggatacagcagtttattactgtaacgcaagaagtatttggagagattattggggtcaaggtactcaagtgaccgttagttcagcggccgcacatcatcatcaccatcat

SEQ ID NO: 341 - DNA, 435 bp, Artificial Sequence
  sec_41D01_His
atggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcat

SEQ ID NO: 342 - DNA, 375 bp, Artificial Sequence
  cyto_41D01_His
atgcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcat

SEQ ID NO: 343 - DNA, 474 bp, Artificial Sequence
  56F11_His_KDEL
atggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcatggtgctgctaaggatgagctt

SEQ ID NO: 344 - DNA, 1149 bp, Artificial Sequence
  sec_56F11_hinge_Fc_His
atggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcacctagaatccctaagccttctacccctcctggatcttcttgtcctcctggaaatatcctcggaggaccttcagtgttcatcttcccacctaagcctaaggatgctctcatgatctcactcacccctaaggttacatgcgttgtggtggatgtgtctgaggatgatcctgatgtgcacgtgtcatggttcgtggataacaaagaggtgcacactgcttggactcagcctagagaagctcagtacaactctaccttcagggtggtgtctgctctccctatccaacaccaagattggatgaggggtaaagagttcaagtgcaaggtgaacaacaaggctctccctgctcctatcgagaggactatctctaaacctaagggaagggctcagacccctcaagtgtatacaattcctccacctagggaacagatgtctaagaagaaggtttcactcacttgcctcgtgaccaacttcttcagtgaggctatctctgttgagtgggagaggaatggtgagcttgagcaggattacaagaacacccctcctatcctcgattctgatggaacctacttcctctactctaagctcaccgtggataccgattcttggttgcagggtgagatcttcacttgctctgttgtgcatgaggctctccacaaccatcacacccagaagaacctcagtagatctcctggtaaagcggccgcacatcatcatcaccatcat

SEQ ID NO: 345 - DNA, 843 bp, Artificial Sequence
  sec_56F11-9GS-56F11_His
atggcaaacaagcttttcttggtgtgcgctaccttcgctctttgcttccttttgactaacgctcaggttcaattgcaagagtctggtggtggactcgtgcaatctggtggatctcttagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcaggtggtggtggaagtggtggtggttctcaagttcaactgcaggaatcaggtggtggattggttcagtctggtggttctctcaggttgtcatgcgttcacagtaagactactttcactaggaacgcaatgggatggtataggcaagcacttggtaaagagagggaactcgttgcaactatcacaagtggtggaaccactaattacgcagatagtgttaagggaagattcactattagtatggatagtgcaaagaacactgtttacttgcaaatgaactcattgaagccagaggatacagcagtttattactgtaatgttaacactagaagaattttcggtggtactgtgagagagtattggggacagggaacccaggttacagttagttcagcggccgcacatcatcaccatcaccat

SEQ ID NO: 346 - DNA, 453 bp, Artificial Sequence
  sec_56F11_His
atggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcat

SEQ ID NO: 347 - DNA, 393 bp, Artificial Sequence
  cyto_56F11_His
atgcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcat

SEQ ID NO: 348 - protein, 6 aa, Artificial Sequence
  His
HHHHHH

SEQ ID NO: 349 - protein, 4 aa, Artificial Sequence
  ER retention signal
KDEL

SEQ ID NO: 350 - protein, 21 aa, Artificial Sequence
  2S2 secretion signal
MANKLFLVCATFALCFLLTNA

SEQ ID NO: 351 - protein, 217 aa, Artificial Sequence
  Fc region
PGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 352 - protein, 9 aa, Artificial Sequence
  9GS spacer
GGGGSGGGS

SEQ ID NO: 353 - protein, 15 aa, Artificial Sequence
  hinge
PRIPKPSTPPGSSCP

SEQ ID NO: 354 - DNA, 1027 bp, Artificial Sequence
  CaMV 35S promoter
actagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcga

SEQ ID NO: 355 - DNA, 18 bp, Artificial Sequence
  pMA_FW primer
ttgtaaaacgacggccag

SEQ ID NO: 356 - DNA, 20 bp, Artificial Sequence
  pMA_REV primer
ggaaacagctatgaccatgt

SEQ ID NO: 357 - DNA, 36 bp, Artificial Sequence
  pMA_CYTO primer
ccggaattcccaccatgcaggttcagctgcaggaat

SEQ ID NO: 358 - DNA, 11159 bp, Artificial Sequence
  pK7WG2
ggccctctagaggatccccgggtaccctcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgacctgcaggcggccgcactagtgatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 359 - DNA, 9966 bp, Artificial Sequence
  pK7WG2-35S41D01_His_KDEL
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcatggtgctgctaaggatgagctttgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 360 - DNA, 10641 bp, Artificial Sequence
  pK7WG2-35Ssec_41D01_hinge_Fc_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcacctagaatccctaagccttctacccctcctggatcttcttgtcctcctggaaatatcctcggtggaccttctgtgttcatcttcccacctaagcctaaggatgctctcatgatctcactcacccctaaggttacatgcgttgtggtggatgtgtctgaggatgatcctgatgtgcacgtgtcatggttcgtggataacaaagaggtgcacactgcttggactcagcctagagaagctcagtacaactctaccttcagggtggtgtctgctctccctatccaacaccaagattggatgaggggtaaagagttcaagtgcaaggtgaacaacaaggctctccctgctcctatcgagaggactatctctaaacctaagggaagggctcagacccctcaagtgtatacaattcctccacctagggaacagatgtctaagaagaaggtttcactcacttgcctcgtgaccaacttcttcagtgaggctatctctgttgagtgggagaggaatggtgagcttgagcaggattacaagaacacccctcctatcctcgattctgatggaacctacttcctctactctaagctcaccgtggataccgattcttggttgcagggtgagatcttcacttgctctgttgtgcatgaggctctccacaaccatcacacccagaagaacctcagtagatctcctggtaaagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 361 - DNA, 10317 bp, Artificial Sequence
  pK7WG2-35S sec_41D01-9GS-41D01_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcaattgcaagagtctggtggtggacttgttcaggctggtggatctcttagactttcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcaggtggtggtggaagtggtggtggttctcaagttcaactgcaggaatcaggtggtggattggtgcaagctggtggttctttgaggttgtcatgtgctgctagtggtaggaccttcagtaggtacggtatgggatggtttagacagttgcctggtaagcagagggaactcgtgacttcaatcactagaggtggaaccactacttacgcagatagtgttaagggaagattcactatcagtagagataatgcaaagaacactgtttacttgcaaatgaactcattgaagccagaggatacagcagtttattactgtaacgcaagaagtatttggagagattattggggtcaaggtactcaagtgaccgttagttcagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 362 - DNA, 9945 bp, Artificial Sequence
  pK7WG2-35S sec_41D01_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 363 - DNA, 9885 bp, Artificial Sequence
  pK7WG2-35S cyto_41D01_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatgcaggttcagctgcaggaatctggtggtggacttgttcaagctggtggatctcttagactctcttgcgctgcttctggaaggaccttctctagatatggaatgggatggttcaggcagctccctggaaaacagagagagcttgttacctctatcaccaggggtggaactaccacctacgctgattctgtgaagggaaggttcaccatctctagggataacgctaagaacaccgtgtacctccagatgaactctctcaagcctgaggataccgctgtgtactactgcaacgctagatctatttggagggattactggggacagggaactcaggtcaccgtttcttcagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 364 - DNA, 9984 bp, Artificial Sequence
  pK7WG2-35S 56F11_His_KDEL
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcatggtgctgctaaggatgagctttgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 365 - DNA, 10659 bp, Artificial Sequence
  pK7WG2-35S sec_56F11_hinge_Fc_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcacctagaatccctaagccttctacccctcctggatcttcttgtcctcctggaaatatcctcggaggaccttcagtgttcatcttcccacctaagcctaaggatgctctcatgatctcactcacccctaaggttacatgcgttgtggtggatgtgtctgaggatgatcctgatgtgcacgtgtcatggttcgtggataacaaagaggtgcacactgcttggactcagcctagagaagctcagtacaactctaccttcagggtggtgtctgctctccctatccaacaccaagattggatgaggggtaaagagttcaagtgcaaggtgaacaacaaggctctccctgctcctatcgagaggactatctctaaacctaagggaagggctcagacccctcaagtgtatacaattcctccacctagggaacagatgtctaagaagaaggtttcactcacttgcctcgtgaccaacttcttcagtgaggctatctctgttgagtgggagaggaatggtgagcttgagcaggattacaagaacacccctcctatcctcgattctgatggaacctacttcctctactctaagctcaccgtggataccgattcttggttgcagggtgagatcttcacttgctctgttgtgcatgaggctctccacaaccatcacacccagaagaacctcagtagatctcctggtaaagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 366 - DNA, 10353 bp, Artificial Sequence
  pK7WG2-35S sec_56F11-9GS-56F11_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcaattgcaagagtctggtggtggactcgtgcaatctggtggatctcttagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcaggtggtggtggaagtggtggtggttctcaagttcaactgcaggaatcaggtggtggattggttcagtctggtggttctctcaggttgtcatgcgttcacagtaagactactttcactaggaacgcaatgggatggtataggcaagcacttggtaaagagagggaactcgttgcaactatcacaagtggtggaaccactaattacgcagatagtgttaagggaagattcactattagtatggatagtgcaaagaacactgtttacttgcaaatgaactcattgaagccagaggatacagcagtttattactgtaatgttaacactagaagaattttcggtggtactgtgagagagtattggggacagggaacccaggttacagttagttcagcggccgcacatcatcaccatcaccattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 367 - DNA, 9963 bp, Artificial Sequence
  pK7WG2-35S sec_56F11_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatggcaaacaagcttttcttggtgtgcgctaccttcgctctttgcttccttttgactaacgctcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg

SEQ ID NO: 368 - DNA, 9903 bp, Artificial Sequence
  pK7WG2-35S cyto_56F11_His
ggccctctagaggatccccgggtaccgcgaattatcatacatgagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttgagctctcccatatggtcgagatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgacctgcaggcggccgcactagtgatatcacaagtttgtacaaaaaagcaggctgaattcccaccatgcaggttcagctgcaggaatctggtggtggacttgttcagtctggtggatctctcagactctcttgcgtgcactctaagaccaccttcaccagaaatgctatgggatggtacagacaggctctcggaaaagagagagagcttgttgctaccatcacctctggtggaactaccaactacgctgattctgtgaagggaaggttcaccatctctatggattctgctaagaacaccgtgtacctccagatgaactctctcaagcctgaggataccgctgtgtactactgcaacgtgaacaccagaaggatcttcggaggaaccgttagagaatactggggacaaggtactcaggtcaccgtttcttcagcggccgcacatcatcatcaccatcattgaggatccacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcg